(19) United States
(12) Patent Application Publication
(10) Pub. No.: US 2024/0196742 A1
(43) Pub. Date: Jun. 13, 2024

(54) LIGHT EMITTING ELEMENT AND HETEROCYCLIC COMPOUND FOR THE SAME

(71) Applicant: Samsung Display Co., Ltd., Yongin-si (KR)

(72) Inventors: HEECHOON AHN, Yongin-si (KR); YOUNGJIN PARK, Yongin-si (KR); HYUNAH UM, Yongin-si (KR); YESEUL LEE, Yongin-si (KR); JIYONG CHOI, Yongin-si (KR)

(21) Appl. No.: 18/359,602

(22) Filed: Jul. 26, 2023

(30) Foreign Application Priority Data
Oct. 19, 2022 (KR) 10-2022-0135116

(51) Int. Cl. H10K 85/60; C07D 209/86; C09K 11/06

(52) U.S. Cl. CPC H10K 85/6572; C07D 209/86; C09K 11/06; H10K 50/11

(57) ABSTRACT

A light emitting element of one or more embodiments includes a first electrode, a second electrode provided on the first electrode, and at least one functional layer provided between the first electrode and the second electrode. At least one functional layer may include a heterocyclic compound represented by Formula 1 below. The light emitting element of one or more embodiments including the heterocyclic compound of one or more embodiments may exhibit long lifespan characteristics. In some embodiments, the light emitting element of one or more embodiments may have reduced driving voltage.

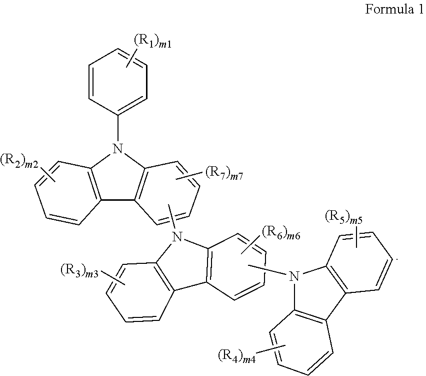

### CROSS-REFERENCE TO RELATED APPLICATION

This application claims priority to and the benefit of Korean Patent Application No. 10-2022-0135116, filed on Oct. 19, 2022, in the Korean Intellectual Property Office, the entire content of which is hereby incorporated by reference.

### BACKGROUND

### 1. Field

One or more aspects of embodiments of the present disclosure herein relate to a light emitting element and a heterocyclic compound used therein.

### 2. Description of the Related Art

As image display devices, organic electroluminescence display devices and/or the like have been actively and lately developed. The organic electroluminescence display devices and/or the like are display devices including, e.g., self-luminescent light emitting elements in which holes and electrons injected, from a first electrode and a second electrode, recombine in an emission layer, and thus a luminescent material in the emission layer emits light to accomplish display of images.

For application of light emitting elements to display devices, there is a demand (or desire) for greater lifespan, and development of materials for light emitting elements capable of stably or suitably attaining such characteristics is being continuously required (or desired).

### SUMMARY

One or more aspects of embodiments of the present disclosure are directed toward a light emitting element having reduced driving voltage and increased service life.

One or more aspects of embodiments of the present disclosure are also directed toward a heterocyclic compound as a material for a light emitting element, which can reduce driving voltage and increase service life.

One or more embodiments of the present disclosure provide a light emitting element including a first electrode, a second electrode provided on the first electrode, and at least one functional layer provided between the first electrode and the second electrode and containing a heterocyclic compound represented by Formula 1 below.

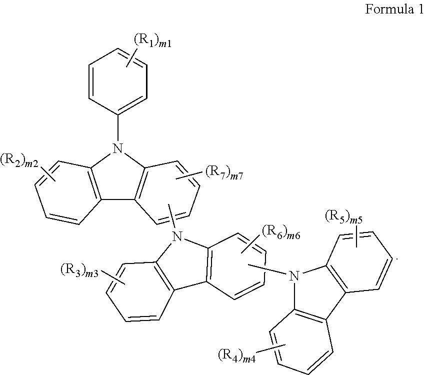

In Formula 1 above, m1 may be an integer of 1 to 5, m2 to m5 may be each independently an integer of 1 to 4, m6 and m7 may be each independently an integer of 1 to 3, and at least one selected from among R₁ to R₇ may be a deuterium atom and the others of R₁ to R₇ may be each independently a hydrogen atom or a substituted or unsubstituted aryl group having 6 to 30 ring-forming carbon atoms.

In one or more embodiments, Formula 1 above may be represented by any one selected from among Formulae 1-1 to 1-3 below.

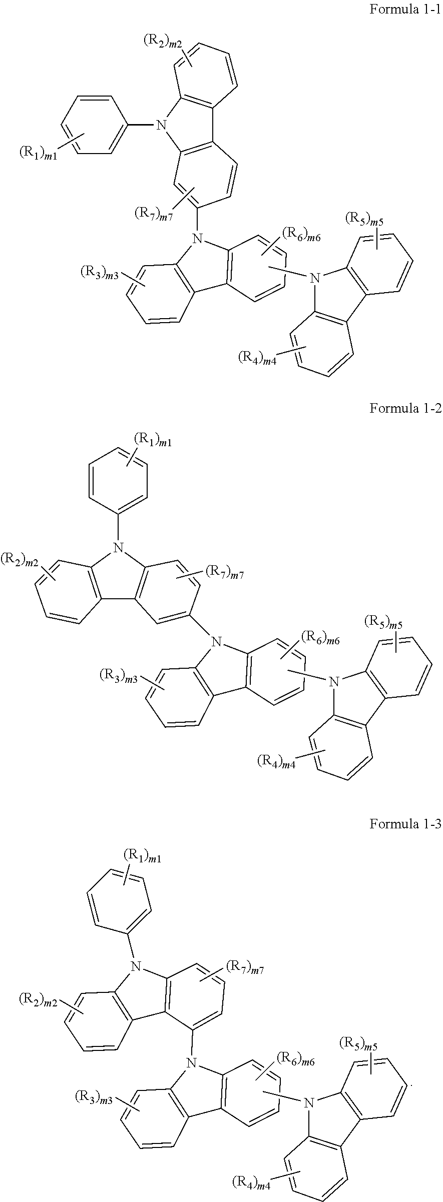

In Formulae 1-1 and 1-3 above, m1 to m7 and R₁ to R₇ are the same as defined in Formula 1 above.

In one or more embodiments, Formula 1-1 above may be represented by any one selected from among Formulae 1-1A to 1-1C below.

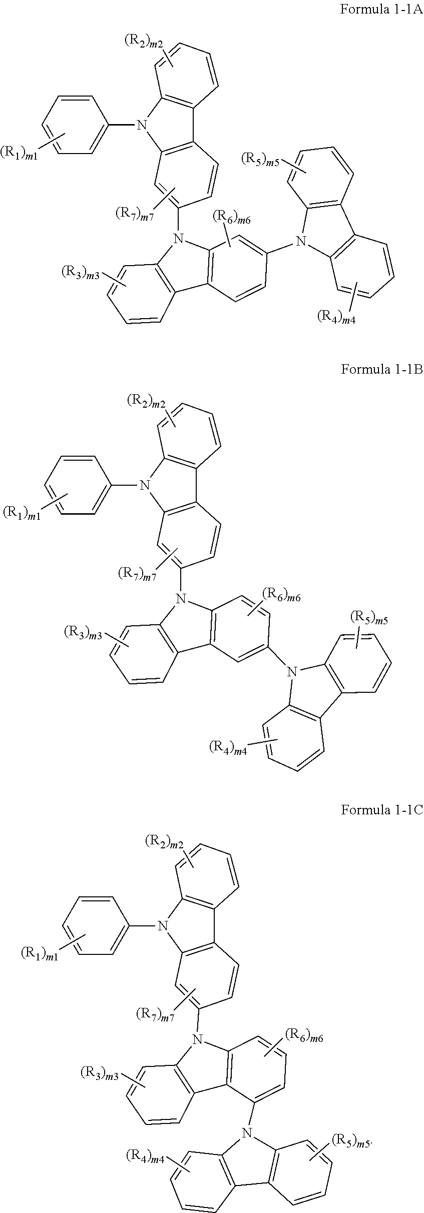

In Formulae 1-1A and 1-1C above, m1 to m7 and R₁ to R₇ are the same as defined in Formula 1-1 above.

In one or more embodiments, Formula 1-2 above may be represented by Formula 1-2A or Formula 1-2B below.

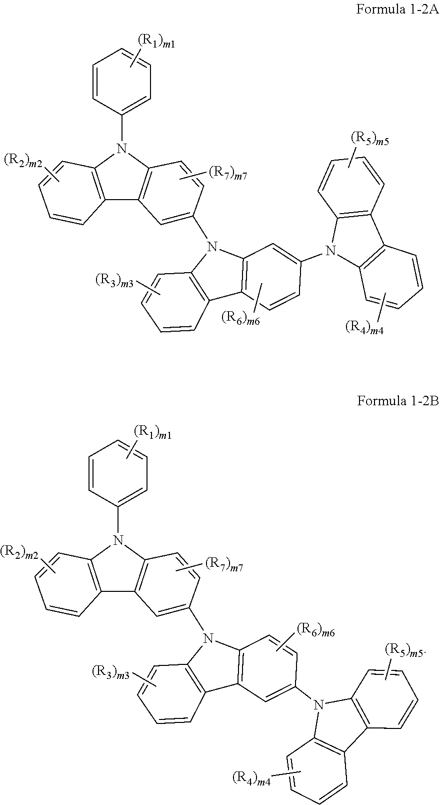

In Formulae 1-2A and 1-2B above, m1 to m7 and R₁ to R₇ are the same as defined in Formula 1-2 above.

In one or more embodiments, Formula 1-3 above may be represented by Formula 1-3A or Formula 1-3B below.

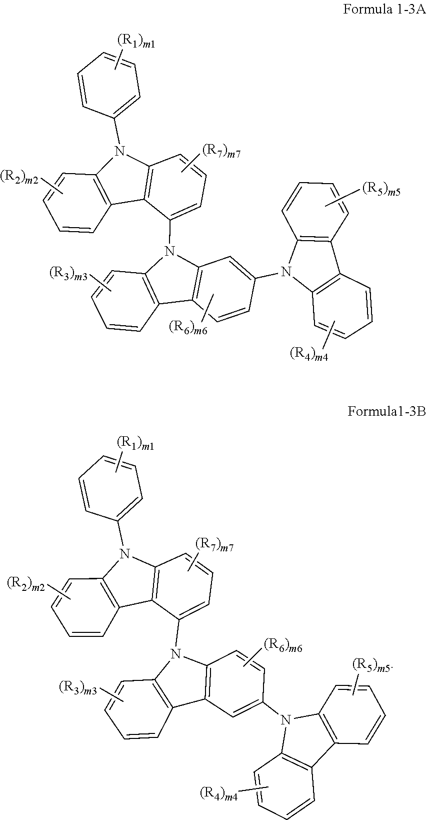

In Formulae 1-3A and 1-3B above, m1 to m7 and R₁ to R₇ are the same as defined in Formula 1-3 above.

In one or more embodiments, Formula 1 above may be represented by Formula 1-X1 or Formula 1-X2 below.

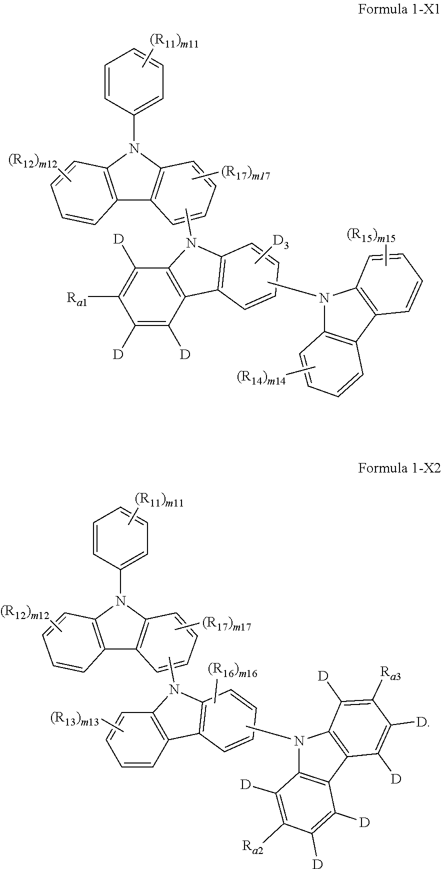

In Formula 1-X1 above, Rₐ₁ may be a deuterium atom or a substituted or unsubstituted phenyl group, m14 and m15 may be each independently an integer of 1 to 4, R₁₄ and R₁₅ may be each independently a hydrogen atom, a deuterium atom, or a substituted or unsubstituted phenyl group; in Formula 1-X2 above, Rₐ₂ and Rₐ₃ may be each independently a deuterium atom or a substituted or unsubstituted phenyl group, m13 may be an integer of 1 to 4, m16 may be an integer of 1 to 3, R₁₃ and R₁₆ may be each independently a hydrogen atom, a deuterium atom, or a substituted or unsubstituted phenyl group; in Formulae 1-X1 and 1-X2 above, D may be a deuterium atom, m11 may be an integer of 1 to 5, m12 may be an integer of 1 to 4, m17 may be an integer of 1 to 3, and R₁₁, R₁₂, and R₁₇ may be each independently a hydrogen atom, a deuterium atom, or a substituted or unsubstituted phenyl group.

In one or more embodiments, Formula 1 above may be represented by Formula 1-X3 below.

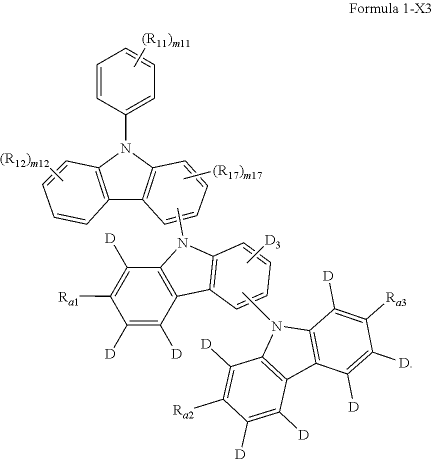

In Formula 1-X3 above, D may be a deuterium atom, m11 may be an integer of 1 to 5, m12 may be an integer of 1 to 4, m17 may be an integer of 1 to 3, R₁₁, R₁₂, and R₁₇ may be each independently a hydrogen atom, a deuterium atom, or a substituted or unsubstituted phenyl group, and Rₐ₁ to Rₐ₃ may be each independently a deuterium atom or a substituted or unsubstituted phenyl group.

In one or more embodiments, the at least one functional layer may include an emission layer, a hole transport region provided between the first electrode and the emission layer, and at least one of the emission layer or the hole transport region may include the heterocyclic compound.

The emission layer may include a dopant and a host, and the host may contain the heterocyclic compound.

In one or more embodiments, the hole transport region may include a hole injection layer provided on the first electrode, a hole transport layer provided on the hole injection layer, and an electron blocking layer provided on the hole transport layer, and at least one of the hole injection layer, the hole transport layer, or the electron blocking layer may include the heterocyclic compound.

In one or more embodiments of the present disclosure, provided is a heterocyclic compound represented by Formula 1 above.

In one or more embodiments, in Formula 1-X3, at least one selected from among Rₐ₁ to Rₐ₃ may be an unsubstituted phenyl group or a phenyl group substituted with a deuterium atom.

### BRIEF DESCRIPTION OF THE DRAWINGS

The accompanying drawings are included to provide a further understanding of the present disclosure, and are incorporated in and constitute a part of this specification. The drawings illustrate embodiments of the present disclosure and, together with the description, serve to explain principles of the present disclosure. In the drawings:

FIG. 1 is a plan view showing a display device according to one or more embodiments,

FIG. 2 is a cross-sectional view showing a portion corresponding to line I-I′ of FIG. 1;

FIG. 3 is a cross-sectional view schematically showing a light emitting element according to one or more embodiments;

FIG. 4 is a cross-sectional view schematically showing a light emitting element according to one or more embodiments;

FIG. 5 is a cross-sectional view schematically showing a light emitting element according to one or more embodiments;

FIG. 6 is a cross-sectional view schematically showing a light emitting element according to one or more embodiments;

FIG. 7 is a cross-sectional view schematically showing a light emitting element according to one or more embodiments;

FIG. 8 is a cross-sectional view showing a display device according to one or more embodiments;

FIG. 9 is a cross-sectional view showing a display device according to one or more embodiments;

FIG. 10 is a cross-sectional view showing a display device according to one or more embodiments.

FIG. 11 is a cross-sectional view showing a display device according to one or more embodiments; and

FIG. 12 is a view showing a vehicle in which a display device according to one or more embodiments is provided.

### DETAILED DESCRIPTION

The present disclosure may be modified in many alternate forms, and thus specific embodiments will be exemplified in the drawings and described in more detail. It should be understood, however, that it is not intended to limit the present disclosure to the particular forms disclosed, but rather, is intended to cover all modifications, equivalents, and alternatives falling within the spirit and scope of the present disclosure.

As used herein, when an element (or a region, a layer, a portion, and the like) is referred to as being “on,” “connected to,” or “coupled to” another element, it means that the element may be directly provided on/connected to/coupled to the other element (e.g., without any intervening elements therebetween), or that an intervening third element may be provided therebetween.

Like reference numerals refer to like elements. In addition, in the drawings, the thickness, the ratio, and the dimensions of elements are exaggerated for an effective description of technical contents. The term “and/or,” includes all combinations of one or more of which associated configurations may define.

It will be understood that, although the terms “first”, “second”, and the like may be used herein to describe various elements, these elements should not be limited by these terms. These terms are only used to distinguish one element from another. For example, a first element could be termed a second element, and, similarly, a second element could be termed a first element, without departing from the scope of example embodiments of the present disclosure. The terms of a singular form may include plural forms unless the context clearly indicates otherwise.

In addition, terms such as “below,” “lower,” “above,” “upper,” and the like are used to describe the relationship of the configurations shown in the drawings. The terms are used as a relative concept and are described with reference to the direction indicated in the drawings.

It should be understood that the terms “comprise”, or “have” are intended to specify the presence of stated features, integers, steps, operations, elements, components, or combinations thereof in the disclosure, but do not preclude the presence or addition of one or more other features, integers, steps, operations, elements, components, or combinations thereof.

As used herein, the terms “use,” “using,” and “used” may be considered synonymous with the terms “utilize,” “utilizing,” and “utilized,” respectively.

As used herein, expressions such as “at least one of”, “one of”, and “selected from”, when preceding a list of elements, modify the entire list of elements and do not modify the individual elements of the list. For example, “at least one selected from among a, b and c”, “at least one of a, b or c”, and “at least one of a, b and/or c” may indicate only a, only b, only c, both (e.g., simultaneously) a and b, both (e.g., simultaneously) a and c, both (e.g., simultaneously) b and c, all of a, b, and c, or variations thereof.

Further, the use of “may” when describing embodiments of the present disclosure refers to “one or more embodiments of the present disclosure”.

As used herein, the terms “substantially”, “about”, and similar terms are used as terms of approximation and not as terms of degree, and are intended to account for the inherent deviations in measured or calculated values that would be recognized by those of ordinary skill in the art. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The electronic device and/or any other relevant devices or components according to embodiments of the present invention described herein may be implemented utilizing any suitable hardware, firmware (e.g. an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

Unless otherwise defined, all terms (including technical and scientific terms) used herein have the same meaning as commonly understood by one of ordinary skill in the art to which the present disclosure pertains. It is also to be understood that terms defined in commonly used dictionaries should be interpreted as having meanings consistent with the meanings in the context of the related art, and are expressly defined herein unless they are interpreted in an ideal or overly formal sense.

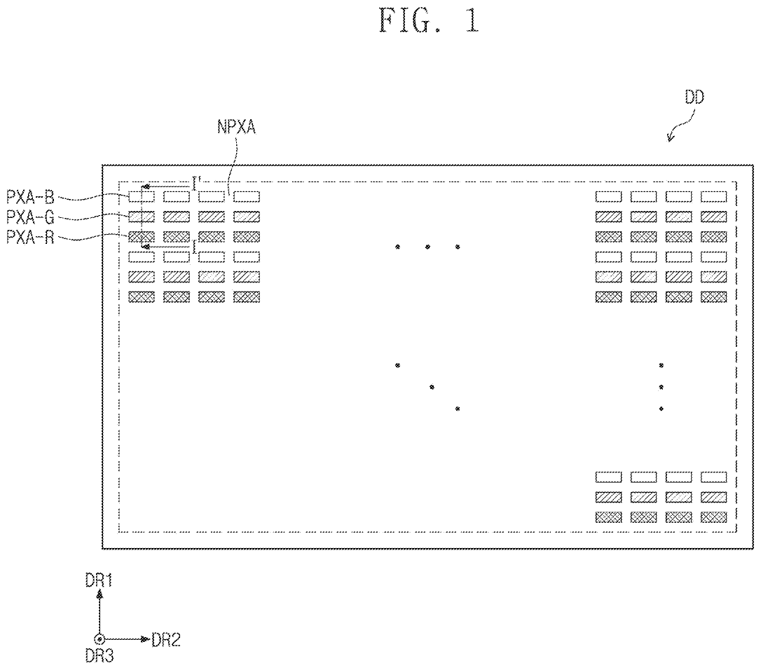

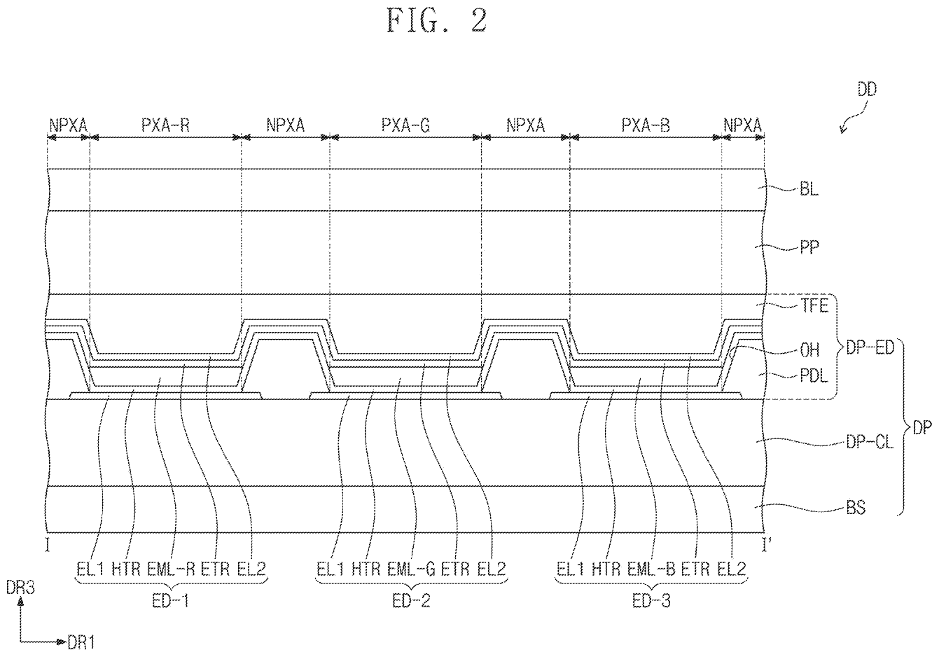

Hereinafter, embodiments of the present disclosure will be described with reference to the accompanying drawings. FIG. 1 is a plan view showing one or more embodiments of a display device DD. FIG. 2 is a cross-sectional view of a display device DD of one or more embodiments. FIG. 2 is a cross-sectional view showing a portion corresponding to line I-I′ of FIG. 1.

A display device DD may include a display panel DP and an optical layer PP provided on the display panel DP. The display panel DP may include light emitting elements ED-1, ED-2, and ED-3. The display device DD may include a plurality of light emitting elements ED-1, ED-2, and ED-3. The optical layer PP may be provided on the display panel DP to control reflected light in the display panel DP due to external light. The optical layer PP may include, for example, a polarizing layer and/or a color filter layer. In some embodiments, the optical layer PP may not be provided (e.g., may be omitted) in the display device DD of one or more embodiments.

A base substrate BL may be provided on the optical layer PP. The base substrate BL may be a member providing a base surface on which the optical layer PP is provided. The base substrate BL may be a glass substrate, a metal substrate, a plastic substrate, and/or the like. However, the embodiment of the present disclosure is not limited thereto, and the base substrate BL may be an inorganic layer, an organic layer, or a composite material layer (e.g., including an organic material and an inorganic material). In some embodiments, the base substrate BL may not be provided (e.g., may be omitted).

The display device DD according to one or more embodiments may further include a filling layer. The filling layer may be provided between a display element layer DP-ED and the base substrate BL. The filling layer may be an organic material layer. The filling layer may include at least one selected from among an acrylic resin, a silicone-based resin, and an epoxy-based resin.

The display panel DP may include a base layer BS, a circuit layer DP-CL provided on the base layer BS, and a display element layer DP-ED. The display element layer DP-ED may include pixel defining films PDL, a plurality of light emitting elements ED-1, ED-2, and ED-3 provided between the pixel defining films PDL, and an encapsulation layer TFE provided on the plurality of light emitting elements ED-1, ED-2, and ED-3.

The base layer BS may be a member providing a base surface in which the display element layer DP-ED is provided. The base layer BS may be a glass substrate, a metal substrate, a plastic substrate, and/or the like. However, the embodiment of the present disclosure is not limited thereto, and the base layer BS may be an inorganic layer, an organic layer, or a composite material layer (e.g., including an organic material and an inorganic material).

In one or more embodiments, the circuit layer DP-CL may be provided on the base layer BS, and the circuit layer DP-CL may include a plurality of transistors. The transistors may each include a control electrode, an input electrode, and an output electrode. For example, the circuit layer DP-CL may include a switching transistor and a driving transistor for driving the plurality of light emitting elements ED-1, ED-2 and ED-3 of the display element layer DP-ED.

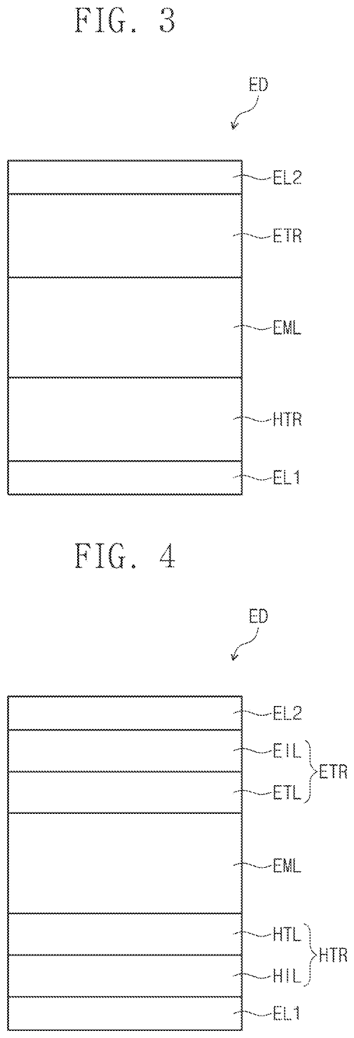

The light emitting elements ED-1, ED-2, and ED-3 may each have a structure of a light emitting element ED according to one or more embodiments of FIGS. 3 to 7, which will be described in more detail herein below. The light emitting elements ED-1, ED-2, and ED-3 may each include a first electrode EL1, a hole transport region HTR, a corresponding one of emission layers EML-R, EML-G, and EML-B, an electron transport region ETR, and a second electrode EL2.

FIG. 2 shows one or more embodiments in which the emission layers EML-R, EML-G, and EML-B of the light emitting elements ED-1, ED-2, and ED-3 are provided in openings OH defined in the pixel defining films PDL, and the hole transport region HTR, the electron transport region ETR, and the second electrode EL2 are provided as a common layer throughout the light emitting elements ED-1, ED-2, and ED-3. However, the embodiment of the present disclosure is not limited thereto, and in one or more embodiments, the hole transport region HTR and the electron transport region ETR may be provided to be patterned inside the openings OH defined in the pixel defining films PDL. For example, in one or more embodiments, the hole transport region HTR, the emission layers EML-R, EML-G, and EML-B, and the electron transport region ETR, and/or the like of the light emitting elements ED-1, ED-2, and ED-3 may be patterned and provided through an inkjet printing method.

The encapsulation layer TFE may cover the light emitting elements ED-1, ED-2 and ED-3. The encapsulation layer TFE may seal the display element layer DP-ED. The encapsulation layer TFE may be a thin film encapsulation layer. The encapsulation layer TFE may be a single layer or a laminated layer of a plurality of layers. The encapsulation layer may include at least one insulating layer. The encapsulation layer TFE according to one or more embodiments may include at least one inorganic film (hereinafter, an encapsulation inorganic film). In some embodiments, the encapsulation layer TFE according to one or more embodiments may include at least one organic film (hereinafter, an encapsulation organic film) and at least one encapsulation inorganic film.

The encapsulation inorganic film may protect the display element layer DP-ED from moisture/oxygen, and the encapsulation organic film may protect the display element layer DP-ED from foreign substances such as dust particles. The encapsulation inorganic film may include silicon nitride, silicon oxy nitride, silicon oxide, titanium oxide, aluminum oxide, and/or the like, but is not particularly limited thereto.

The encapsulation organic film may include an acrylic compound, an epoxy-based compound, and/or the like. The encapsulation organic film may include a photopolymerizable organic material, and is not particularly limited.

The encapsulation layer TFE may be provided on the second electrode EL2, and may be provided to fill the openings OH.

Referring to FIGS. 1 and 2, the display device DD may include non-light emitting regions NPXA and light emitting regions PXA-R, PXA-G, and PXA-B. The light emitting regions PXA-R, PXA-G, and PXA-B may each be a region emitting (e.g., configured to emit) light generated from each of the light emitting elements ED-1, ED-2, and ED-3. The light emitting regions PXA-R, PXA-G, and PXA-B may be spaced apart from each other when viewed on a plane (e.g., in a plan view).

The light emitting regions PXA-R, PXA-G, and PXA-B may each be a region separated by the pixel defining films PDL. The non-light emitting regions NPXA may be regions between neighboring light emitting regions PXA-R, PXA-G, and PXA-B, and may correspond to the pixel defining films PDL. In some embodiments, as used herein, the light emitting regions PXA-R, PXA-G, and PXA-B may each correspond to a pixel. The pixel defining film PDL may separate the light emitting elements ED-1, ED-2 and ED-3. The emission layers EML-R, EML-G, and EML-B of the light emitting elements ED-1, ED-2 and ED-3 may be provided and separated in openings OH defined by the pixel defining films PDL.

The light emitting regions PXA-R, PXA-G, and PXA-B may be divided into a plurality of groups according to the color of light generated from the light emitting elements ED-1, ED-2, and ED-3. In the display device DD of one or more embodiments shown in FIGS. 1 and 2, three light emitting regions PXA-R, PXA-G, and PXA-B which emit (or are configured to emit) red light, green light, and blue light, are shown as an example. For example, the display device DD of one or more embodiments may include a red light emitting region PXA-R, a green light emitting region PXA-G, and a blue light emitting region PXA-B, which are distinct from one another.

In the display device DD according to one or more embodiments, the plurality of light emitting elements ED-1, ED-2, and ED-3 may emit light having different wavelength ranges. For example, in one or more embodiments, the display device DD may include a first light emitting element ED-1 emitting red light, a second light emitting element ED-2 emitting green light, and a third light emitting element ED-3 emitting blue light. For example, the red light emitting region PXA-R, the green light emitting region PXA-G, and the blue light emitting region PXA-B of the display device DD may correspond to the first light emitting element ED-1, the second light emitting element ED-2, and the third light emitting element ED-3, respectively.

However, the embodiment of the present disclosure is not limited thereto, and the first to third light emitting elements ED-1, ED-2 and ED-3 may emit light in the same wavelength range or emit light in at least one different wavelength range. For example, the first to third light emitting elements ED-1, ED-2, and ED-3 all may emit blue light.

The light emitting regions PXA-R, PXA-G, and PXA-B in the display device DD according to one or more embodiments may be arranged in the form of a stripe. Referring to FIG. 1, a plurality of red light emitting regions PXA-R may each be arranged with each other along a second directional axis DR2, a plurality of green light emitting regions PXA-G may each be arranged with each other along a second directional axis DR2, and a plurality of blue light emitting regions PXA-B may each be arranged with each other along a second directional axis DR2. In some embodiments, the red light emitting region PXA-R, the green light emitting region PXA-G, and the blue light emitting region PXA-B may be arranged alternately with each (in turn) along a first directional axis DR1.

FIGS. 1 and 2 show that the light emitting regions PXA-R, PXA-G, and PXA-B are all similar in size, but the embodiment of the present disclosure is not limited thereto, and the light emitting regions PXA-R, PXA-G and PXA-B may be different in size from each other according to wavelength range of emitted light. In some embodiments, the areas of the light emitting regions PXA-R, PXA-G, and PXA-B may refer to areas when viewed on a plane defined by the first directional axis DR1 and the second directional axis DR2.

In some embodiments, the arrangement of the light emitting regions PXA-R, PXA-G, and PXA-B is not limited to what is shown in FIG. 1, and the order in which the red light emitting region PXA-R, the green light emitting region PXA-G, and the blue light emitting region PXA-B are arranged may suitably vary according to display quality characteristics required (or desired) for the display device DD. For example, the light emitting regions PXA-R, PXA-G, and PXA-B may be arranged in the form of a pentile (PENTILE® is a registered trademark owned by Samsung Display Co., Ltd.) or a diamond (Diamond Pixel™)

In some embodiments, areas of each of the light emitting regions PXA-R, PXA-G, and PXA-B may be different in size from one another. For example, in one or more embodiments, the green light emitting region PXA-G may be smaller than the blue light emitting region PXA-B in size, but the embodiment of the present disclosure is not limited thereto.

Hereinafter, FIGS. 3 to 7 are cross-sectional views schematically showing a light emitting element according to one or more embodiments. The light emitting element ED according to one or more embodiments may include a first electrode EL1, a hole transport region HTR, an emission layer EML, an electron transport region ETR, and a second electrode EL2.

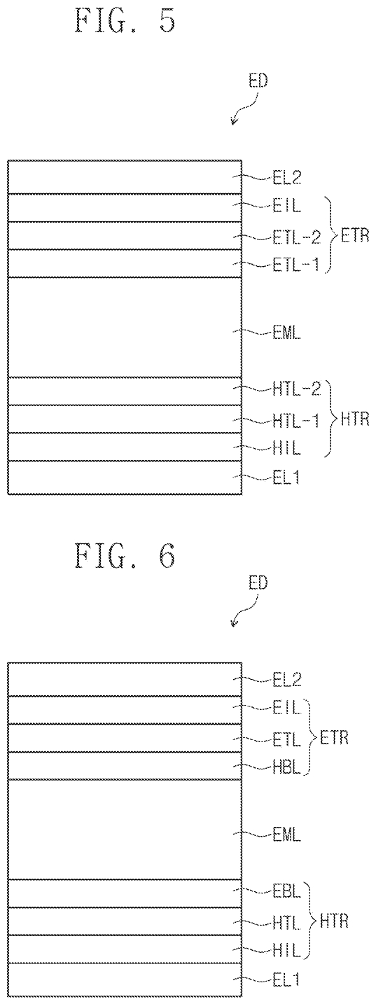

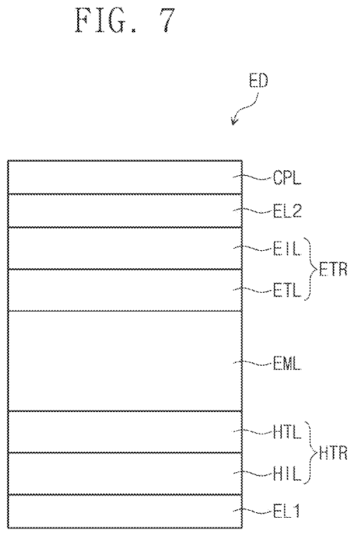

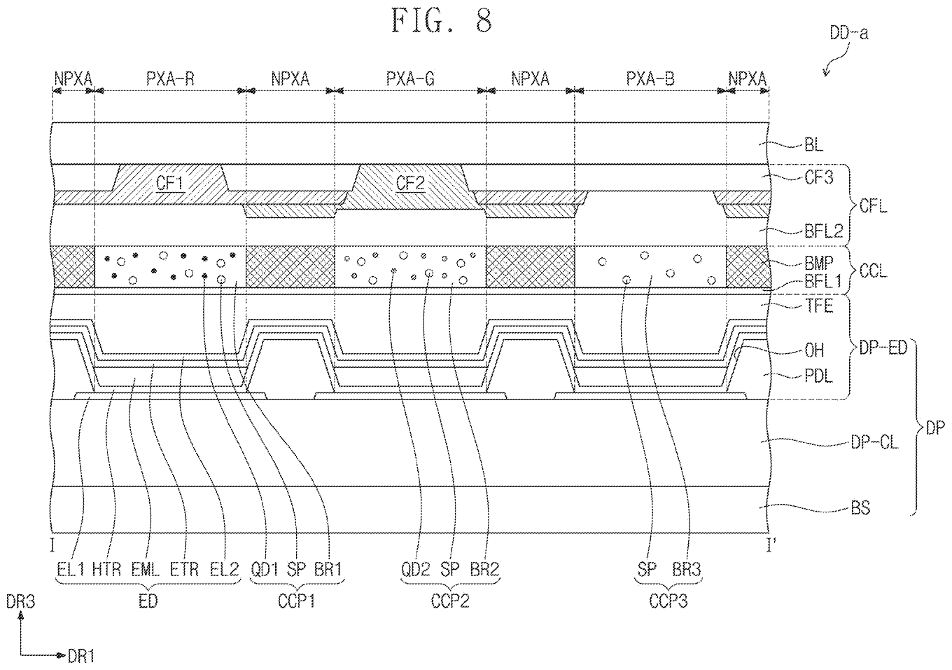

FIG. 4 shows, compared with FIG. 3, a cross-sectional view of a light emitting element ED of one or more embodiments in which the hole transport region HTR includes a hole injection layer HIL and a hole transport layer HTL, and the electron transport region ETR includes an electron injection layer EIL and an electron transport layer ETL. In some embodiments, FIG. 5 shows, compared with FIG. 3, a cross-sectional view of a light emitting element ED of one or more embodiments in which the hole transport region HTR includes a hole injection layer HIL, a first hole transport layer HTL-1, and a second hole transport layer HTL-2, and the electron transport region ETR includes an electron injection layer EIL, a second electron transport layer ETL-2, and a first electron transport layer ETL-1. FIG. 6 shows, compared with FIG. 3, a cross-sectional view of a light emitting element ED according to one or more embodiments in which the hole transport region HTR includes a hole injection layer HIL, a hole transport layer HTL, and an electron blocking layer EBL, and the electron transport region ETR includes an electron injection layer EIL, an electron transport layer ETL, and a hole blocking layer HBL. FIG. 7 shows, compared with FIG. 4, a cross-sectional view of a light emitting element ED according to one or more embodiments, in which a capping layer CPL is provided on the second electrode EL2.

The light emitting element ED of one or more embodiments may include the heterocyclic compound of one or more embodiments in at least one functional layer provided between a first electrode and a second electrode. At least one functional layer may include a hole transport region HTR, an emission layer EML, and/or an electron transport region ETR. For example, at least one of the hole transport region HTR or the emission layer EML may include the heterocyclic compound of one or more embodiments. The heterocyclic compound of one or more embodiments may include three carbazole groups, and at least one of the three carbazole groups may include at least one deuterium atom directly bonded to a carbazole group. The light emitting element ED including the heterocyclic compound of one or more embodiments may exhibit long lifespan. In some embodiments, the light emitting element ED including the heterocyclic compound of one or more embodiments may have reduced driving voltage. The heterocyclic compound of one or more embodiments will be described in more detail with reference to Formula 1.

As used herein, the term “substituted or unsubstituted” may indicate a group that is unsubstituted or that is substituted with at least one substituent selected from the group consisting of a deuterium atom, a halogen atom, a cyano group, a nitro group, an amine group, a silyl group, oxy group, thio group, sulfinyl group, sulfonyl group, carbonyl group, a boron group, a phosphine oxide group, a phosphine sulfide group, an alkyl group, an alkenyl group, an alkynyl group, a hydrocarbon ring group, an aryl group, a heterocyclic group, and combinations thereof. In some embodiments, each of the substituents presented as an example above may be substituted or unsubstituted. For example, a biphenyl group may be interpreted as an aryl group or as a phenyl group substituted with a phenyl group.

As used herein, the term “bonded to an adjacent group to form a ring” may indicate that a group is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocyclic or polycyclic. In some embodiments, the rings formed by being bonded to each other may be connected to another ring to form a spiro structure.

As used herein, the term “an adjacent group” may refer to a pair of substituent groups where the first substituent is connected to an atom which is directly connected to another atom substituted with the second substituent; a pair of substituent groups connected to the same atom; or a pair of substituent groups where the first substituent is sterically positioned at the nearest position to the second substituent. For example, two methyl groups in 1,2-dimethylbenzene may be interpreted as mutually “adjacent groups” and two ethyl groups in 1,1-diethylcyclopentane may be interpreted as mutually “adjacent groups”. In some embodiments, two methyl groups in 4,5-dimethylphenanthrene may be interpreted as mutually “adjacent groups”.

As used herein, examples of a halogen atom may include a fluorine atom, a chlorine atom, a bromine atom, and an iodine atom.

As used herein, an alkyl group may be a linear, branched or cyclic group. The number of carbon atoms in the alkyl group is not particularly limited but may be 1 to 60, 1 to 50, 1 to 30, 1 to 20, 1 to 10, or 1 to 6. Examples of the alkyl group may include a methyl group, an ethyl group, an n-propyl group, an isopropyl group, an n-butyl group, a s-butyl group, a t-butyl group, an i-butyl group, a 2-ethylbutyl group, a 3,3-a dimethylbutyl group, an n-pentyl group, an i-pentyl group, a neopentyl group, a t-pentyl group, a Cyclopentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, a cyclohexyl group, a 4-methylcyclohexyl group, a 4-t-butylcyclohexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, a Cyclooctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldodecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, and the like, but are not limited thereto.

As used herein, an alkenyl group refers to a hydrocarbon group including at least one carbon double bond in the middle and/or end of an alkyl group having 2 or more carbon atoms. The alkenyl group may be linear or branched. The number of carbon atoms is not particularly limited, but may be 2 to 60, 2 to 30, 2 to 20, or 2 to 10. Examples of the alkenyl group may include a vinyl group, a 1-butenyl group, a 1-pentenyl group, a 1,3-butadienyl group, a styrenyl group, a styryl vinyl group, and the like, but are not limited thereto.

As used herein, an alkynyl group refers to a hydrocarbon group including at least one carbon triple bond in the middle and/or end of an alkyl group having 2 or more carbon atoms. The alkynyl group may be linear or branched. The number of carbon atoms is not particularly limited, but may be 2 to 60, 2 to 30, 2 to 20, or 2 to 10. Examples of the alkynyl group may include an ethynyl group, a propynyl group, etc., but are not limited thereto.

In the present description, a hydrocarbon ring group refers to any functional group or substituent derived from an aliphatic hydrocarbon ring. The hydrocarbon ring group may be a saturated hydrocarbon ring group having 5 to 20 ring-forming carbon atoms.

As used herein, an aryl group refers to any functional group or substituent derived from an aromatic hydrocarbon ring. The aryl group may be a monocyclic aryl group or a polycyclic aryl group. The number of ring-forming carbon atoms in the aryl group may be 6 to 60, 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, and the like, but are not limited thereto.

In the present description, a heterocyclic group refers to any functional group or substituent derived from a ring containing at least one of B, O, N, P, Si, Se, Te, or S as a hetero atom. The heterocyclic group includes an aliphatic heterocyclic group and an aromatic heterocyclic group. The aromatic heterocyclic group may be a heteroaryl group. The aliphatic heterocyclic group and the aromatic heterocyclic group may be monocyclic or polycyclic.

In the present description, the heterocyclic group may contain at least one of B, O, N, P, Si, Se, Te, or S as a hetero atom. When the heterocyclic group contains two or more hetero atoms, the two or more hetero atoms may be the same as or different from each other. The heterocyclic group may be a monocyclic heterocyclic group or a polycyclic heterocyclic group, and may include a heteroaryl group. The number of ring-forming carbon atoms in the heterocyclic group may be 2 to 60, 2 to 30, 2 to 20, or 2 to 10.

As used herein, the aliphatic heterocyclic group may contain at least one of B, O, N, P, Si, Se, Te, or S as a hetero atom. The number of ring-forming carbon atoms in the aliphatic heterocyclic group may be 2 to 60, 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, and the like, but are not limited to thereto.

In the present description, the heteroaryl group may contain at least one of B, O, N, P, Si, Se, Te, or S as a hetero atom. When the heteroaryl group contains two or more hetero atoms, the two or more hetero atoms may be the same as or different from each other. The heteroaryl group may be a monocyclic heteroaryl group or a polycyclic heteroaryl group. The number of ring-forming carbon atoms in the heteroaryl group may be 2 to 60, 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzoimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, and the like, but are not limited thereto.

As used herein, the above description of the aryl group may be applied to an arylene group, except that the arylene group is a polyvalent group (e.g., a divalent group). The above description of the heteroaryl group may be applied to a heteroarylene group, except that the heteroarylene group is a polyvalent group (e.g., a divalent group).

As used herein, a silyl group may refer to a group in which a silicon atom is bonded to an alkyl group or aryl group as defined above. The silyl group includes an alkyl silyl group and an aryl silyl group. Examples of the silyl group may include a trimethylsilyl group, a triethylsilyl group, a t-butyldimethylsilyl group, an ethyldimethylsilyl group, a propyldimethylsilyl group, a triphenylsilyl group, a diphenylsilyl group, a phenylsilyl group, and the like, but are not limited thereto.

As used herein, the number of carbon atoms in a carbonyl group is not particularly limited, but may be 1 to 40, 1 to 30, or 1 to 20. For example, the carbonyl group may have the following structure, but is not limited thereto:

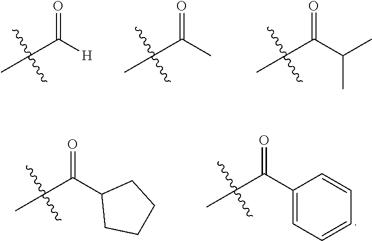

In the present description, the number of carbon atoms in a sulfinyl group and a sulfonyl group is not particularly limited, but may be 1 to 30. The sulfinyl group may include an alkyl sulfinyl group and an aryl sulfinyl group. The sulfonyl group may include an alkyl sulfonyl group and an aryl sulfonyl group.

As used herein, a thio group may include an alkyl thio group and an aryl thio group. The thio group may indicate a group in which a sulfur atom is bonded to an alkyl group or an aryl group as defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, and the like, but are not limited to thereto.

As used herein, an oxy group may indicate a group in which an oxygen atom is bonded to an alkyl group or aryl group as defined above. The oxy group may include an alkoxy group and an aryl oxy group. The alkoxy group may be linear, branched or cyclic. The number of carbon atoms in the alkoxy group is not particularly limited, but may be, for example, 1 to 20, or 1 to 10. Examples of the oxy group may include methoxy, ethoxy, n-propoxy, isopropoxy, butoxy, pentyloxy, hexyloxy, octyloxy, nonyloxy, decyloxy, benzyloxy, and the like, but are not limited thereto.

As used herein, a boron group may refer to a group in which a boron atom is bonded to an alkyl group or aryl group as defined above. The boron group includes an alkyl boron group and an aryl boron group. Examples of the boron group may include a dimethylboron group, a diethylboron group, a t-butylmethylboron group, a diphenylboron group, a phenylboron group, etc., but are not limited thereto.

In the present description, the number of carbon atoms in an amine group is not particularly limited, but may be 1 to 30. The amine group may include an alkyl amine group and an aryl amine group. Examples of the amine group may include a methylamine group, a dimethylamine group, a phenylamine group, a diphenylamine group, a naphthylamine group, a 9-methyl-anthracenylamine group, and the like, but are not limited thereto.

As used herein, the above-described examples of the alkyl group also apply to the alkyl group in an alkylthio group, an alkyl sulfinyl group, an alkyl sulfonyl group, an alkoxy group, an alkyl amine group, an alkyl boron group, an alkyl silyl group, and an alkyl amine group.

As used herein, the above-described examples of the aryl group also apply to the aryl group in an aryloxy group, an arylthio group, an aryl sulfinyl group, an aryl sulfonyl group, an aryl amine group, an aryl boron group, an aryl silyl group, and an aryl amine group.

As used herein, a direct linkage may refer to a chemical bond (e.g., a single bond). In the present description,

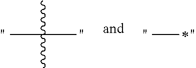

refer to positions to be connected (e.g., a binding site).

The light emitting element ED according to one or more embodiments may include a heterocyclic compound of one or more embodiments. The heterocyclic compound of one or more embodiments may be represented by Formula 1 below.

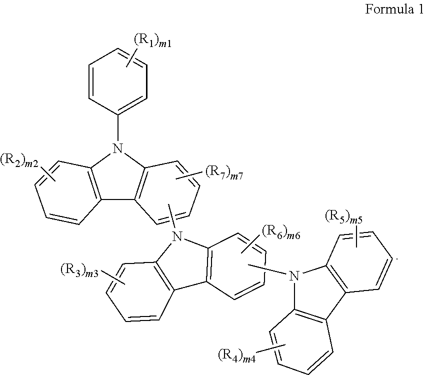

In Formula 1, m1 may be an integer of 1 to 5. When m1 is an integer of 2 or greater, a plurality of R₁'s may all be the same or at least one may be different from the others.

m2 to m5 may each independently be an integer of 1 to 4. When m2 is an integer of 2 or greater, a plurality of R₂'s may all be the same or at least one may be different from the others. When m3 is an integer of 2 or greater, a plurality of R₃'s may all be the same or at least one may be different from the others. When m4 is an integer of 2 or greater, a plurality of R₄'s may all be the same or at least one may be different from the others. When m5 is an integer of 2 or greater, a plurality of R₅'s may all be the same or at least one may be different from the others.

m6 and m7 may each independently be an integer of 1 to 3, and when m6 is an integer of 2 or greater, a plurality of R₆'s may all be the same or at least one may be different from the others. When m7 is an integer of 2 or greater, a plurality of R₇'s may all be the same or at least one may be different from the others.

In one or more embodiments, at least one selected from among R₁ to R₇ may be a deuterium atom, and the others of R₁ to R₇ may each independently be a hydrogen atom, a deuterium atom, or a substituted or unsubstituted aryl group having 6 to 30 ring-forming carbon atoms. For example, R₁ may be a hydrogen atom or a deuterium atom.

The heterocyclic compound of one or more embodiments including at least one deuterium atom may contribute to improving lifespan of the light emitting element ED. The heterocyclic compound of one or more embodiments includes three carbazole groups, and in one carbazole group (hereinafter, a first carbazole group) of the three carbazole groups, a phenyl group is bonded to a nitrogen atom of the first carbazole group, which is a ring-forming atom, and the phenyl group may include R₁ of Formula 1 as a substituent. One of the two benzene rings forming the first carbazole group includes R₂ of Formula 1 as a substituent, and the other benzene ring includes R₇ of Formula 1 as a substituent. In Formula 1, any one of the remaining two carbazole groups (hereinafter referred to as a second carbazole group) may be bonded to the benzene ring of the first carbazole group including R₇ as a substituent.

A nitrogen atom, which is a ring-forming atom in the second carbazole group, may be at a position bonded to the first carbazole group. One of the two benzene rings forming the second carbazole group includes R₃ of Formula 1 as a substituent, and the other benzene ring includes R₆ of Formula 1 as a substituent. In Formula 1, the remaining one carbazole group (hereinafter referred to as a third carbazole group) may be bonded to the benzene ring of the second carbazole group including R₆ as a substituent.

A nitrogen atom, which is a ring-forming atom in the third carbazole group, may be at a position bonded to the second carbazole group. One of the two benzene rings forming the third carbazole group includes R₄ of Formula 1 as a substituent, and the other benzene ring includes R₅ of Formula 1 as a substituent.

In the heterocyclic compound of one or more embodiments, at least one selected from among the first to third carbazole groups may include a deuterium atom as a substituent. The deuterium atom may be directly or indirectly bonded to at least one carbazole group selected from among the first to third carbazole groups. The heterocyclic compound of one or more embodiments includes three carbazole groups and at least one deuterium atom bonded to a carbazole group, and thus has a HOMO (Highest Occupied Molecular Orbital) energy level of −5.6 eV or higher and a lowest triplet excitation (T1) energy level of 2.8 eV or higher, and shows excellent or improved hole transport properties. Accordingly, the light emitting element ED including the heterocyclic compound of one or more embodiments may exhibit long lifespan. In some embodiments, the light emitting element ED including the heterocyclic compound of one or more embodiments may have reduced driving voltage.

In one or more embodiments, Formula 1 may be represented by any one selected from among Formulae 1-1 to 1-3 below. Formulae 1-1 to 1-3 show a specific (defined) bonding position of the second carbazole group to the first carbazole group in Formula 1. As described above, in Formula 1, the first carbazole group includes R₂ and R₇, and the second carbazole group includes R₃ and R₆.

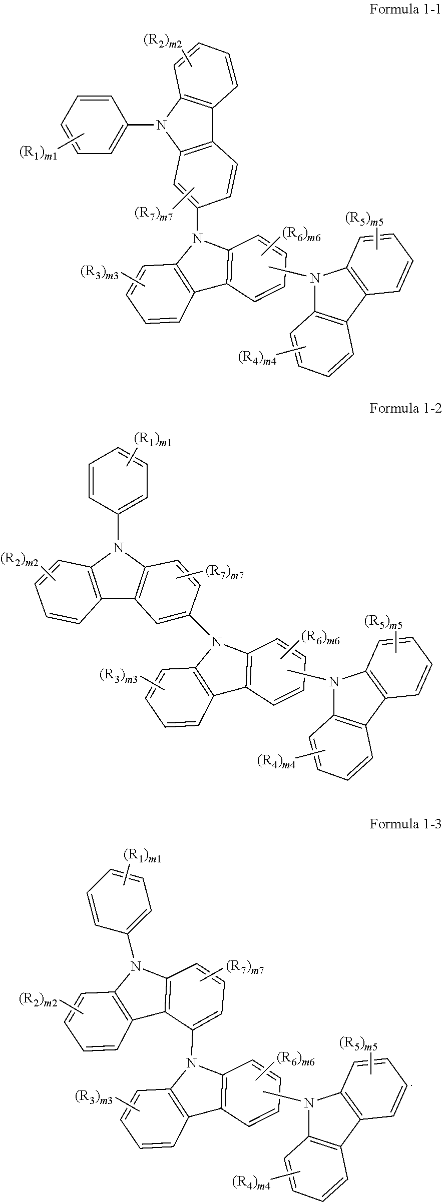

Formula 1-1 shows a case where a nitrogen atom of the second carbazole group is bonded to the carbon atom numbered 2 (C2) of the first carbazole group in Formula 1. Formula 1-2 shows a case where a nitrogen atom of the second carbazole group is bonded to the carbon atom numbered 3 (C3) of the first carbazole group in Formula 1. Formula 1-3 shows a case where a nitrogen atom of the second carbazole group is bonded to the carbon atom numbered 4 (C4) of the first carbazole group in Formula 1. In Formulae 1-1 to 1-3, the same descriptions as in Formula 1 may be applied to m1 to m7 and R₁ to R₇. In some embodiments, carbon atoms as ring-forming atoms of a carbazole group are numbered as follows:

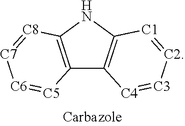

Formula 1-1 may be represented by any one selected from among Formulae 1-1A to 1-1C below. Formulae 1-1A to 1-1C show a specific (or defined) bonding position of the third carbazole group to the second carbazole group in Formula 1-1. As described above, the third carbazole group includes R₄ and R₅.

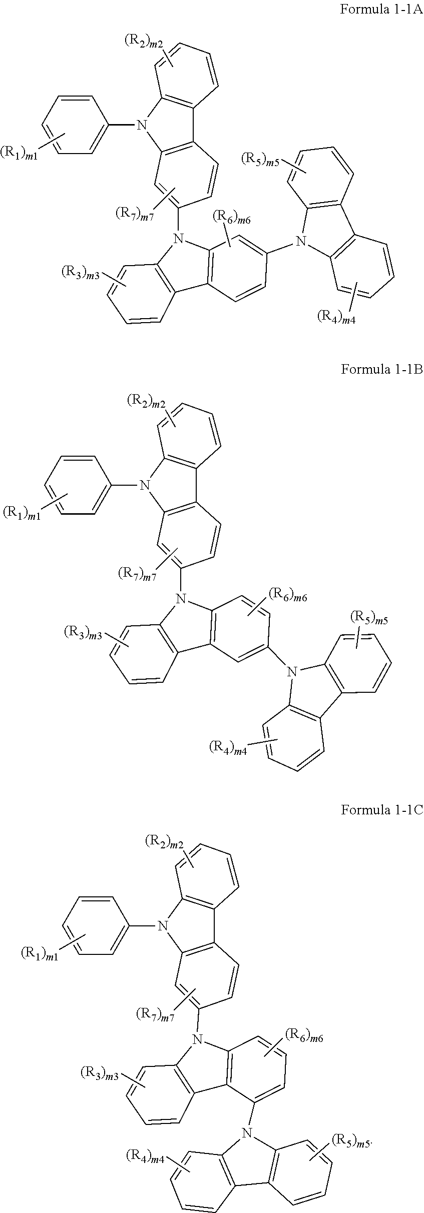

Formula 1-1A shows a case where a nitrogen atom of the third carbazole group is bonded to the carbon atom numbered 2 (C2) of the second carbazole group in Formula 1-1. Formula 1-11B shows a case where a nitrogen atom of the third carbazole group is bonded to the carbon atom numbered 3 (C3) of the second carbazole group in Formula 1-1. Formula 1-1C shows a case where a nitrogen atom of the third carbazole group is bonded to the carbon atom numbered 4 (C4) of the second carbazole group in Formula 1-1. In Formulae 1-1A to 1-1C, the same descriptions as in Formula 1-1 may be applied to m1 to m7 and R₁ to R₇.

Formula 1-2 may be represented by Formula 1-2A or Formula 1-2B below. Formulae 1-2A to 1-2B show a specific (or defined) bonding position of the third carbazole group to the second carbazole group in Formula 1-2.

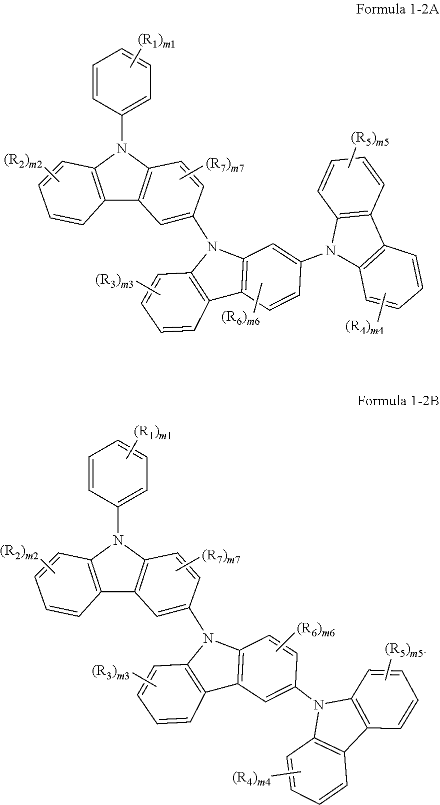

Formula 1-2A shows a case where a nitrogen atom of the third carbazole group is bonded to the carbon atom numbered 2 (C2) of the second carbazole group in Formula 1-2. Formula 1-2B shows a case where a nitrogen atom of the third carbazole group is bonded to the carbon atom numbered 3 (C3) of the second carbazole group in Formula 1-2. In Formulae 1-2A to 1-2B, the same descriptions as in Formula 1-2 may be applied to m1 to m7 and R₁ to R_{7.}

Formula 1-3 may be represented by Formula 1-3A or Formula 1-3B below. Formulae 1-3A and 1-3B show a specific (or defined) bonding position of the third carbazole group to the second carbazole group in Formula 1-3.

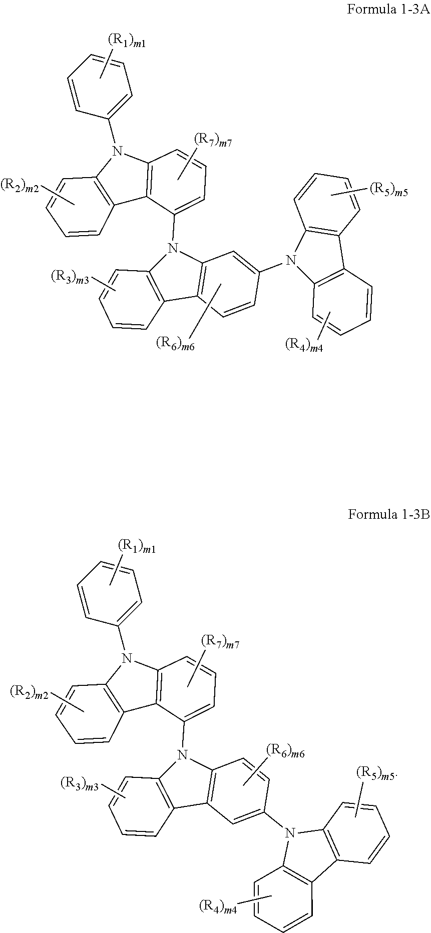

Formula 1-3A shows a case where a nitrogen atom of the third carbazole group is bonded to the carbon atom numbered 2 (C2) of the second carbazole group in Formula 1-3. Formula 1-3B shows a case where a nitrogen atom of the third carbazole group is bonded to the carbon atom numbered 3 (C3) of the second carbazole group in Formula 1-3. In Formulae 1-3A and 1-3B, the same descriptions as in Formula 1-3 may be applied to m1 to m7 and R₁ to R₇.

In one or more embodiments, Formula 1 may be represented by Formula 1-X1 or Formula 1-X2 below. Formulae 1-X1 and 1-X2 show cases in which at least one selected from among R₃ to R₆ in Formula 1 is a deuterium atom. Formula 1-X1 shows a case where m3 is 4, three R₃'s are deuterium atoms, m6 is 3, and three R₆'s are deuterium atoms in Formula 1. Formula 1-X2 shows a case where m4 and m5 are 4, and three R₄'s and three R₅'s are deuterium atoms in Formula 1.

In some embodiments, Formula 1-X1 may show a case in which the second carbazole group in Formula 1 includes a deuterium atom. Formula 1-X2 may show a case in which the third carbazole group in Formula 1 includes a deuterium atom.

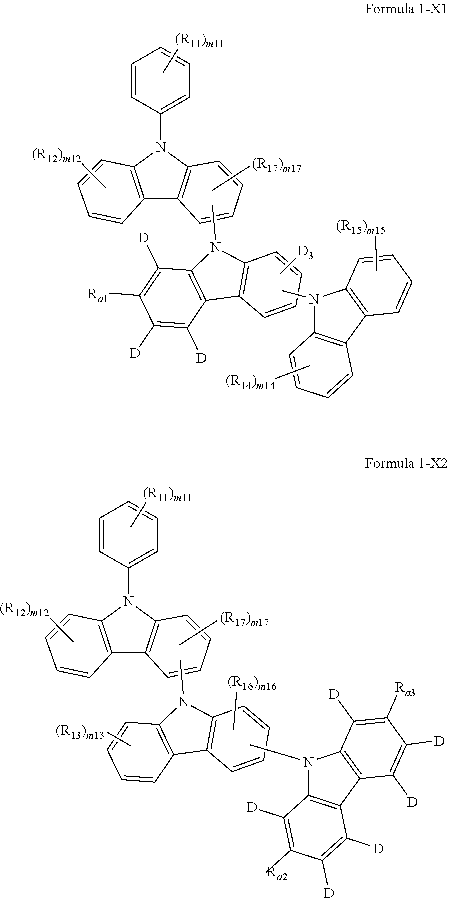

In Formula 1-X1, Rₐ₁ may be a deuterium atom or a substituted or unsubstituted phenyl group. m14 and m15 may each independently be an integer of 1 to 4. When m14 is an integer of 2 or greater, a plurality of R₁₄'s may all be the same or at least one may be different from the others. When m15 is an integer of 2 or greater, a plurality of R₁₅'s may all be the same or at least one may be different from the others. R₁₄ and R₁₅ may each independently be a hydrogen atom, a deuterium atom, or a substituted or unsubstituted phenyl group,

In Formula 1-X2, Rₐ₂ and Rₐ₃ may each independently be a deuterium atom or a substituted or unsubstituted phenyl group. m13 may be an integer of 1 to 4, and m16 may be an integer of 1 to 3. When m13 is an integer of 2 or greater, a plurality of R₁₃'s may all be the same or at least one may be different from the others. When m16 is an integer of 2 or greater, a plurality of R₁₆'s may all be the same or at least one may be different from the others. R₁₃ and R₁₆ may each independently be a hydrogen atom, a deuterium atom, or a substituted or unsubstituted phenyl group.

In Formulae 1-X1 1-X2, D is a deuterium atom, and D3 indicates 3 deuterium atoms.

m11 may be an integer of 1 to 5, m12 may be an integer of 1 to 4, and m17 may be an integer of 1 to 3. When m11 is an integer of 2 or greater, a plurality of R₁₁'s may all be the same or at least one may be different from the others. When m12 is an integer of 2 or greater, a plurality of R₁₂'s may all be the same or at least one may be different from the others. When m17 is an integer of 2 or greater, a plurality of R₁₇'s may all be the same or at least one may be different from the others. R₁₁, R₁₂, and R₁₇ may each independently be a hydrogen atom, a deuterium atom, or a substituted or unsubstituted phenyl group. For example, R₁₁ may be a hydrogen atom or a deuterium atom.

In one or more embodiments, Formula 1 may be represented by Formula 1-X3 below. Formula 1-X3 shows a case in which at least one selected from among R₃ to R₆ in Formula 1 is a deuterium atom. Formula 1-X3 shows a case where m3 to m5 are 4, m6 is 3, and three R₃'s, three R₄'s, three R₅'s, and three R₆'s are deuterium atoms in Formula 1.

In some embodiments, Formula 1-X3 may show a case where m14 and m15 are 4, and three R₁₄'s and three R₁₅'s are deuterium atoms in Formula 1-X1. Formula 1-X3 may show a case where m13 is 4, m16 is 3, and three R₁₃'s and three R₁₆'s are deuterium atoms in Formula 1-X2.

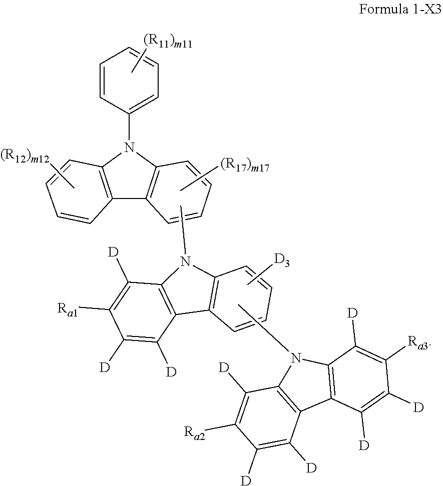

In Formula 1-X3, D is a deuterium atom, and D3 indicates 3 deuterium atoms. m11 may be an integer of 1 to 5, m12 may be an integer of 1 to 4, and m17 may be an integer of 1 to 3. When m11 is an integer of 2 or greater, a plurality of R₁₁'s may all be the same or at least one may be different from the others. When m12 is an integer of 2 or greater, a plurality of R₁₂'s may all be the same or at least one may be different from the others. When m17 is an integer of 2 or greater, a plurality of R₁₇'s may all be the same or at least one may be different from the others.

R₁₁, R₁₂, and R₁₇ may each independently be a hydrogen atom, a deuterium atom, or a substituted or unsubstituted phenyl group, Rₐ₁ to Rₐ₃ may each independently be a deuterium atom or a substituted or unsubstituted phenyl group. For example, at least one selected from among Rₐ₁ to Rₐ₃ may be an unsubstituted phenyl group or a phenyl group substituted with a deuterium atom. In the phenyl group substituted with a deuterium atom, 5 deuterium atoms are directly bonded to the phenyl group.

For example, Formula 1-X3 may be represented by any one selected from among Formulae 1-X31 to 1-X34 below. Formulae 1-X31 to 1-X34 show a specific (or defined) bonding position of the second carbazole group to the first carbazole group and a specific (or defined) bonding position of the third carbazole group to the second carbazole group in Formula 1-X3.

In some embodiments, Formula 1-X31 shows a case where m3 to m5 are 4, m6 is 3, and three R₃'s, three R₄'s, three R₅'s, and three R₆'s are deuterium atoms in Formula 1-1A. Formula 1-X32 shows a case where m3 to m5 are 4, m6 is 3, and three R₃'s, three R₄'s, three R₅'s, and three R₆'s are deuterium atoms in Formula 1-1B.

Formula 1-X33 shows a case where m3 to m5 are 4, m6 is 3, and three R₃'s, three R₄'s, three R₅'s, and three R₆'s are deuterium atoms in Formula 1-2A. Formula 1-X34 shows a case where m3 to m5 are 4, m6 is 3, and three R₃'s, three R₄'s, three R₅'s, and three R₆'s are deuterium atoms in Formula 1-2B.

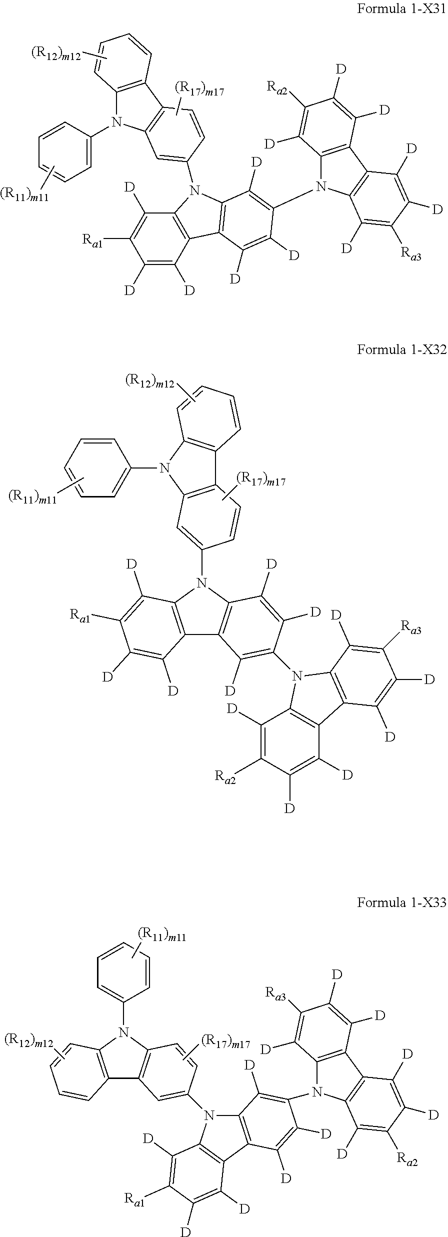

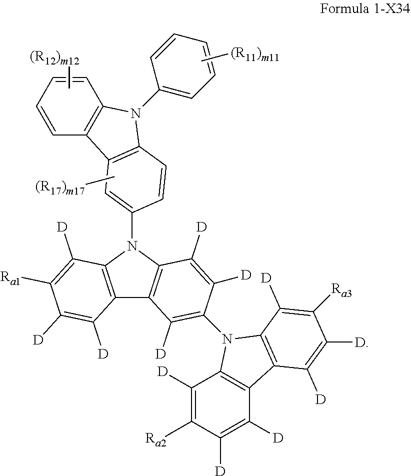

In Formulae 1-X31 to 1-X34, D is a deuterium atom. In Formulae 1-X31 to 1-X34, the same descriptions as in Formula 1-X3 may be applied to m11, m12, m17, R₁₁, R₁₂, R₁₇, and Rₐ₁ to Rₐ₃.

A heterocyclic compound of one or more embodiments may be represented by any one selected from among compounds of Compound Group 1 below. A light emitting element ED of one or more embodiments may include at least one of the compounds from Compound Group 1 below. In Compound Group 1 below, D is a deuterium atom.

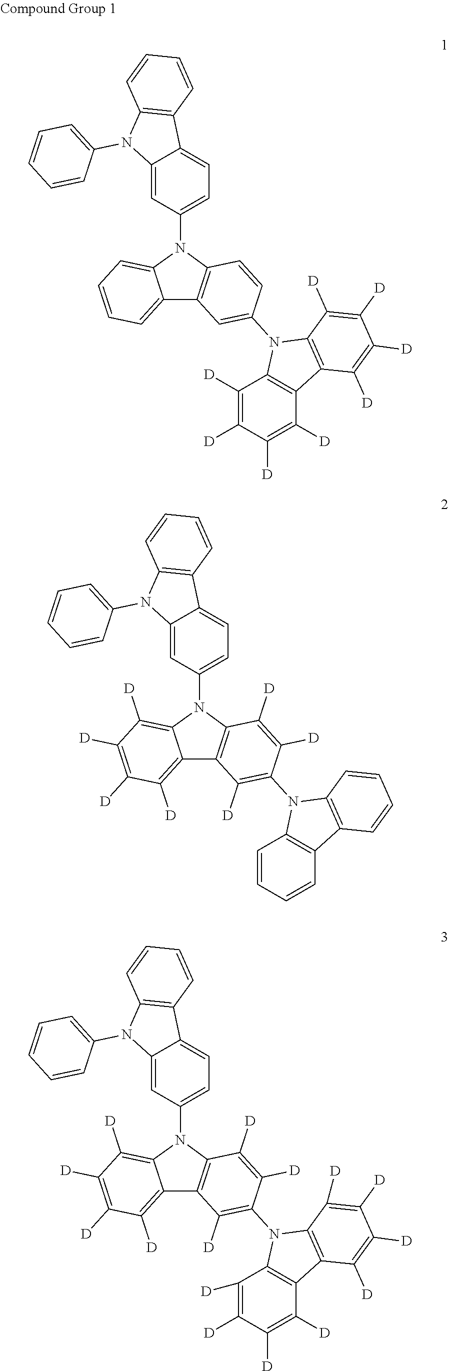 [SMILES: [2H]c1c([2H])c([2H])c2c(c1[2H])c1c([2H])c([2H])c([2H])c([2H])c1n2-c1ccc2c(c1)c1ccccc1n2-c1ccc2c3ccccc3n(-c3ccccc3)c2c1 ; [2H]c1c([2H])c([2H])c2c(c1[2H])c1c([2H])c(-n3c4ccccc4c4ccccc43)c([2H])c([2H])c1n2-c1ccc2c3ccccc3n(-c3ccccc3)c2c1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc2c3ccccc3n(-c3ccccc3)c2c1]

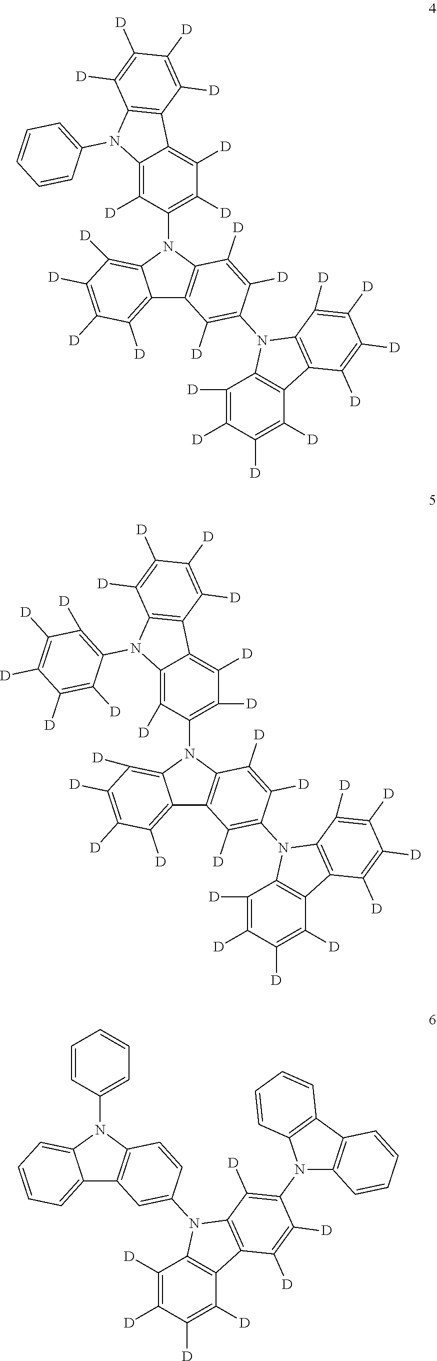

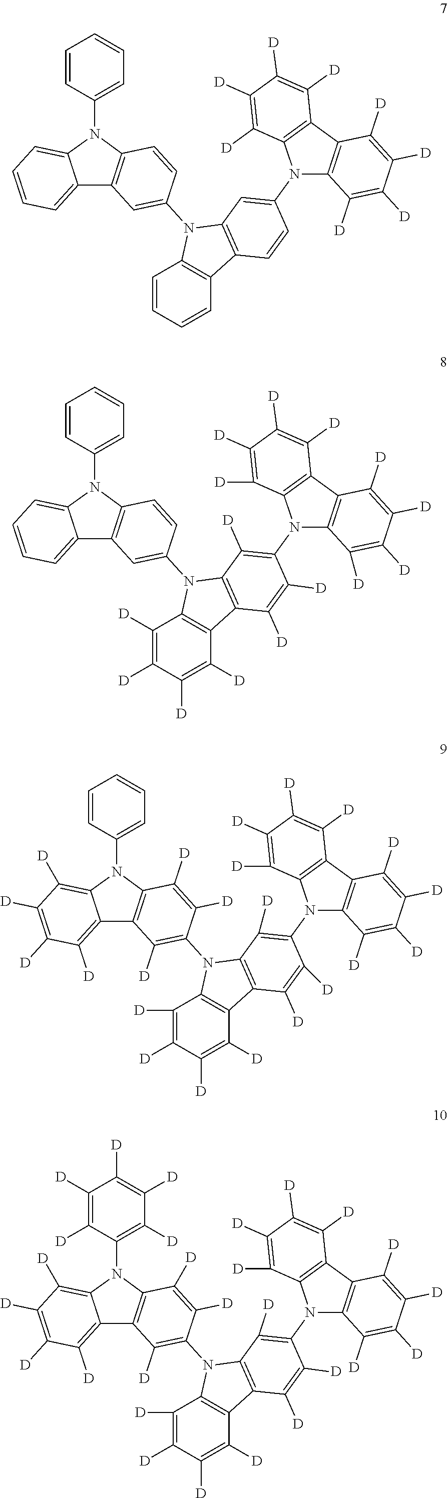

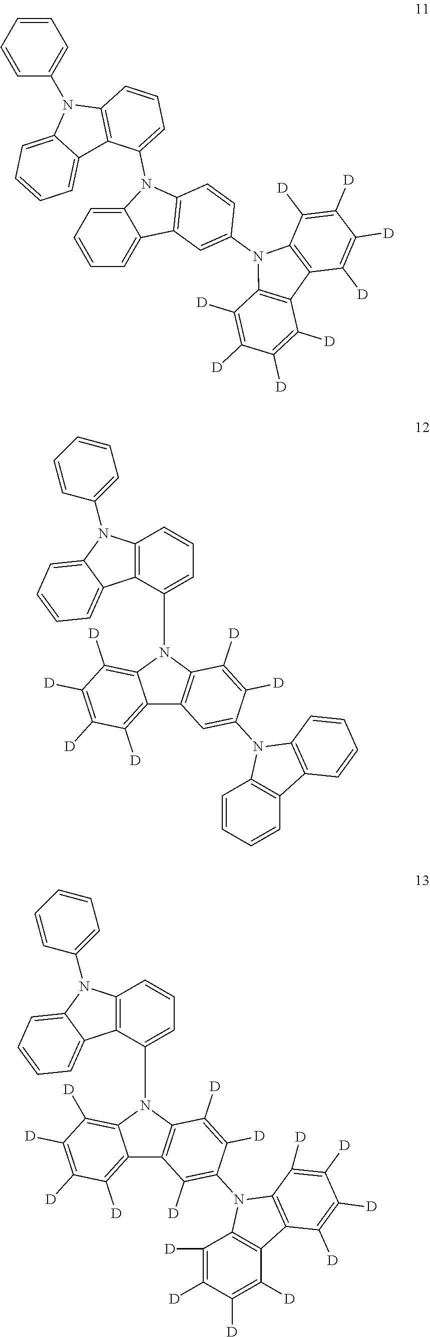 [SMILES: [2H]c1c([2H])c([2H])c2c(c1[2H])c1c([2H])c([2H])c([2H])c([2H])c1n2-c1ccc2c(c1)c1ccccc1n2-c1cccc2c1c1ccccc1n2-c1ccccc1 ; [2H]c1c([2H])c([2H])c2c(c1[2H])c1cc(-n3c4ccccc4c4ccccc43)c([2H])c([2H])c1n2-c1cccc2c1c1ccccc1n2-c1ccccc1 ; [2H]c1c([2H])c([2H])c2c(c1[2H])c1c([2H])c([2H])c([2H])c([2H])c1n2-c1c([2H])c([2H])c2c(c1[2H])c1c([2H])c([2H])c([2H])c([2H])c1n2-c1cccc2c1c1ccccc1n2-c1ccccc1]

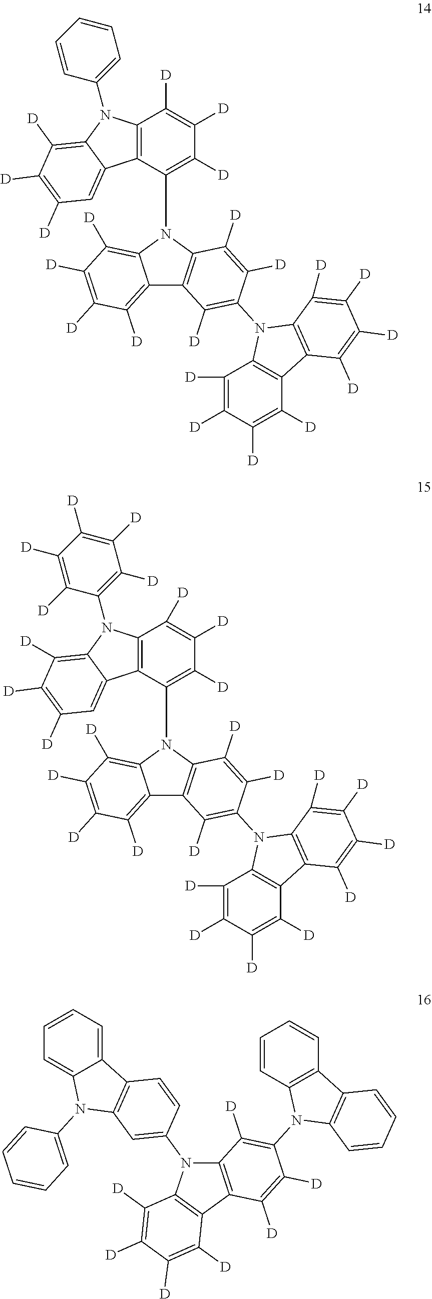

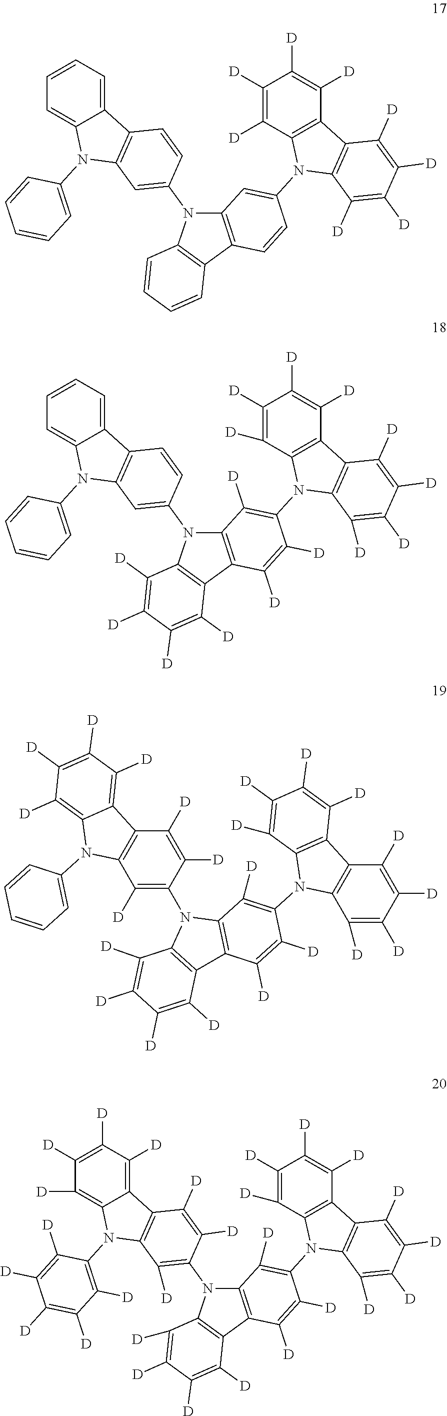

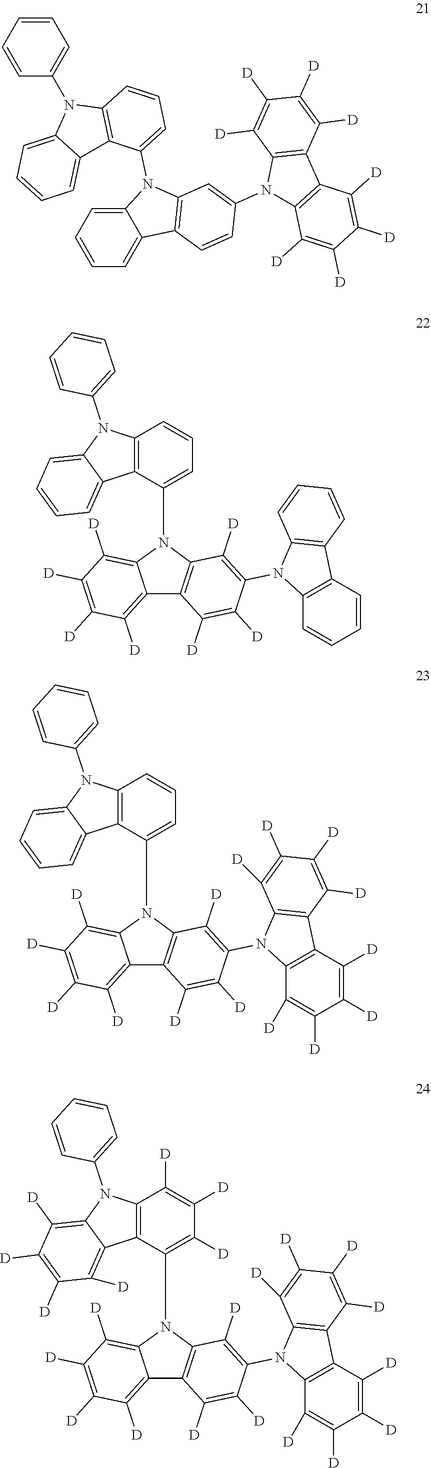

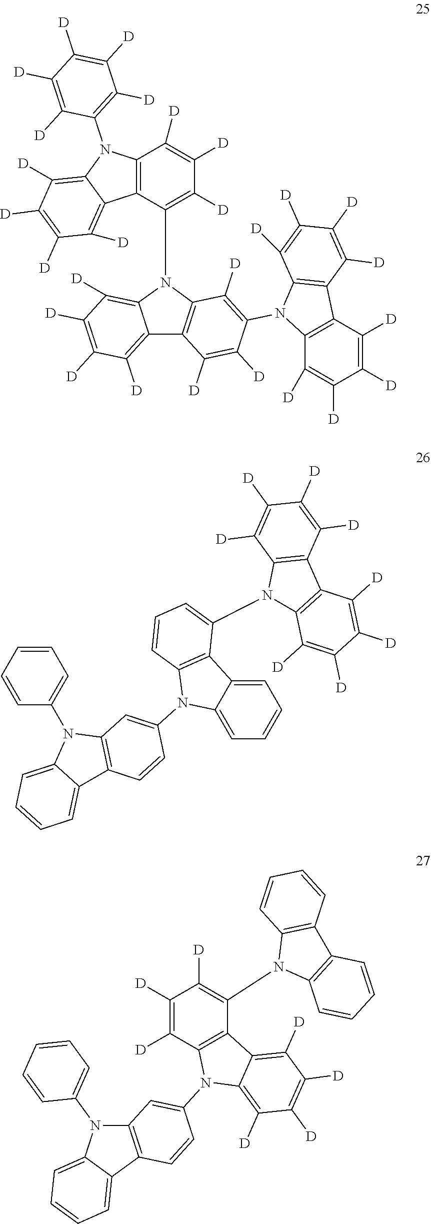 [SMILES: [2H]c1c([2H])c([2H])c(-n2c3c([2H])c([2H])c([2H])c([2H])c3c3c(-n4c5c([2H])c([2H])c([2H])c([2H])c5c5c([2H])c([2H])c(-n6c7c([2H])c([2H])c([2H])c([2H])c7c7c([2H])c([2H])c([2H])c([2H])c76)c([2H])c54)c([2H])c([2H])c([2H])c32)c([2H])c1[2H] ; [2H]c1c([2H])c([2H])c2c(c1[2H])c1c([2H])c([2H])c([2H])c([2H])c1n2-c1cccc2c1c1ccccc1n2-c1ccc2c3ccccc3n(-c3ccccc3)c2c1 ; [2H]c1c([2H])c([2H])c2c(c1[2H])c1c(-n3c4ccccc4c4ccccc43)c([2H])c([2H])c([2H])c1n2-c1ccc2c3ccccc3n(-c3ccccc3)c2c1]

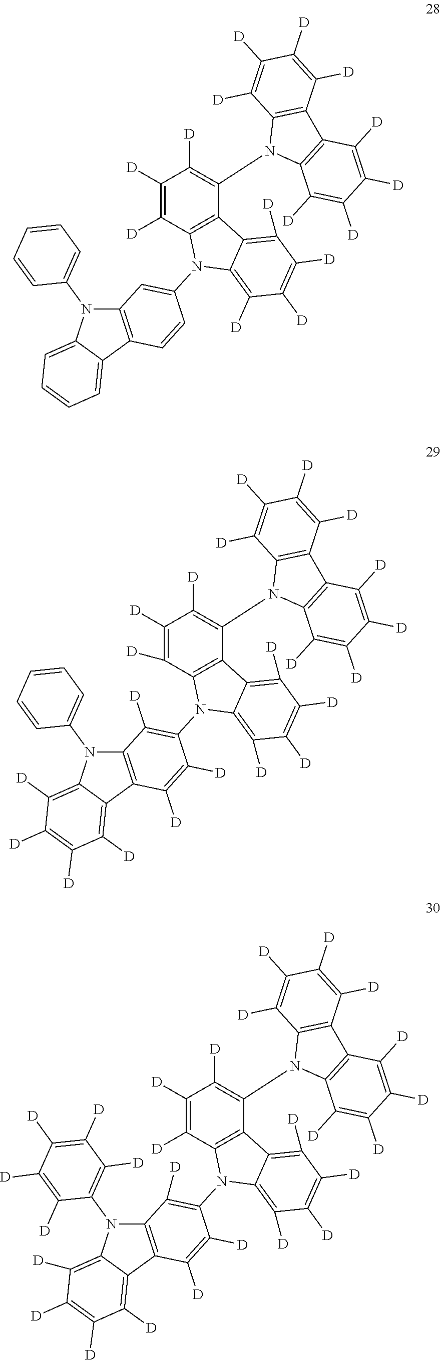

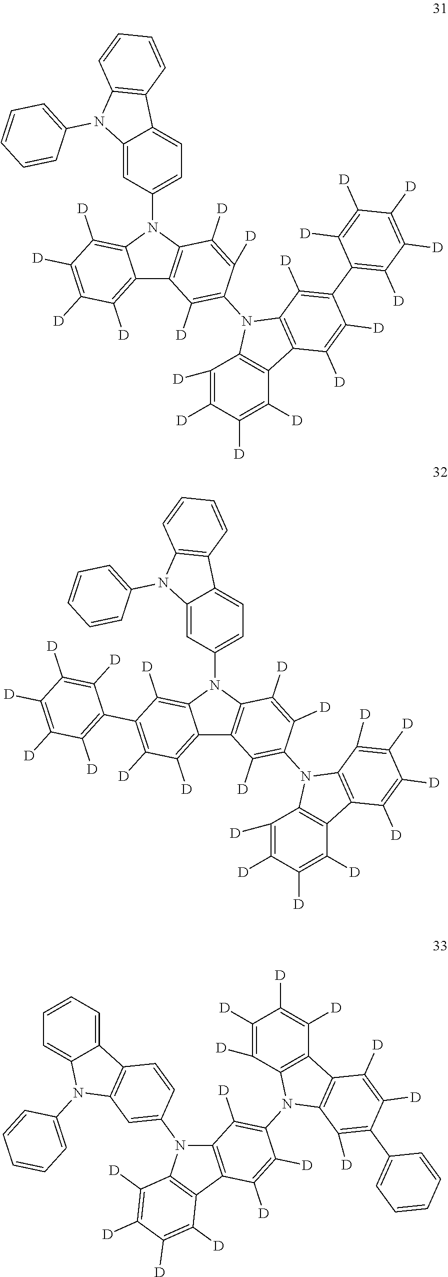

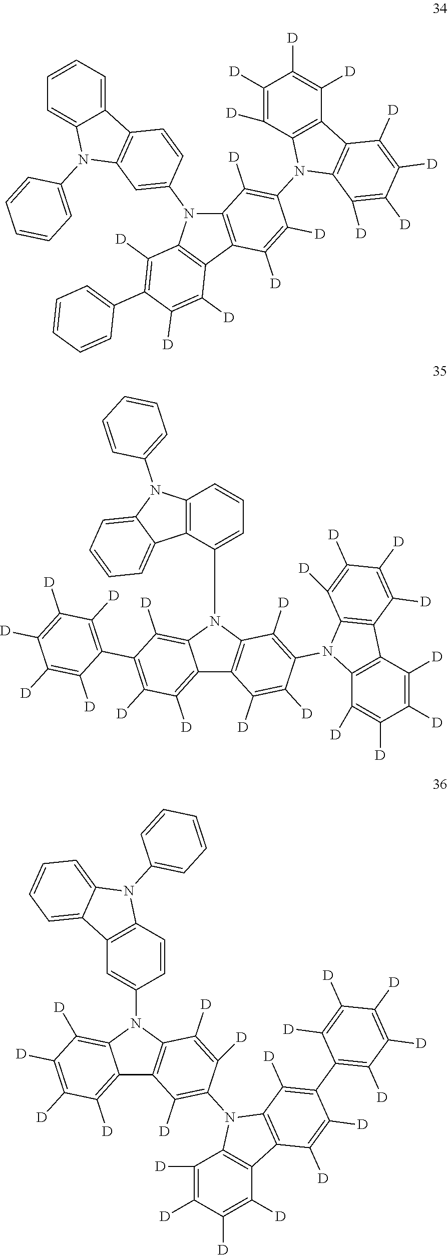

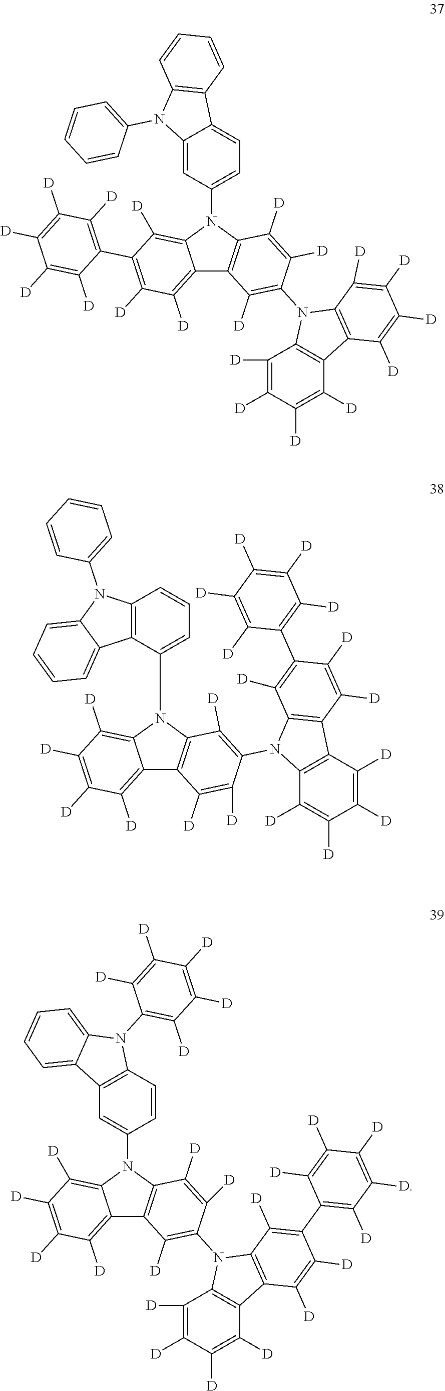

An emission layer EML may emit light of phosphorescence or thermally activated delayed fluorescence (TADF). The emission layer EML may emit blue light. For example, the emission layer EML may emit deep blue light.

In one or more embodiments, the emission layer EML may include a host and a dopant. The heterocyclic compound of one or more embodiments may be included as a host material of the emission layer EML.

In some embodiments, the emission layer EML may include two or more host materials. For example, the emission layer EML may include a hole transporting host material and an electron transporting host material, and the heterocyclic compound of one or more embodiments may be included as the hole transporting host material.

In some embodiments, the emission layer EML may further include a sensitizer. The sensitizer may be provided as a material for helping a dopant emit light, and may include a phosphorescent sensitizer and/or a thermally activated delayed fluorescence sensitizer.

In the emission layer EML, the hole transporting host and the electron transporting host may form an exciplex. In this case, the triplet energy of the exciplex formed by the hole transporting host and the electron transporting host corresponds to a difference between Lowest Unoccupied Molecular Orbital (LUMO) energy level of the electron transporting host and HOMO energy level of the hole transporting host. For example, the triplet energy level (T1) of the exciplex formed by the hole transporting host and the electron transporting host may have an absolute value of about 2.4 eV to about 3.0 eV. In some embodiments, the triplet energy of the exciplex may have a value smaller than the energy gap of each host material. The exciplex may have a triplet energy of 3.0 eV or less, which is an energy gap between the hole transporting host and the electron transporting host. However, this is presented as an example, and the embodiment of the present disclosure is not limited thereto.

When the emission layer EML includes the hole transporting host, the electron transporting host, a sensitizer, and a dopant, the hole transporting host and the electron transporting host may form an exciplex, and energy may be transferred from the exciplex to the sensitizer and from the sensitizer to the dopant, thereby emitting light. However, this is presented as an example, and materials included in the emission layer EML are not limited thereto. In some embodiments, the hole transporting host and the electron transporting host may not form an exciplex. When the hole transporting host and the electron transporting host do not form an exciplex, energy may be transferred from the hole transporting host and the electron transporting host to the sensitizer and from the sensitizer to the dopant, thereby emitting light.

For example, the emission layer EML may include a compound represented by Formula ET-1 below. The compound represented by Formula ET-1 below may be used as an electron transporting host material.

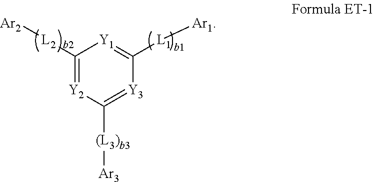

In Formula ET-1, at least one selected from among Y₁ to Y₃ may be N and the others may each independently be CRₐ. Rₐ may be a hydrogen atom, a deuterium atom, a substituted or unsubstituted alkyl group having 1 to 20 carbon atoms, a substituted or unsubstituted aryl group having 6 to 60 ring-forming carbon atoms, or a substituted or unsubstituted heteroaryl group having 2 to 60 ring-forming carbon atoms.

When any one of Y₁ to Y₃ is N, the compound represented by Formula ET-1 may include a pyridine group. When any two of Y₁ to Y₃ are N, the compound represented by Formula ET-1 may include a pyrimidine group. When all of Y₁ to Y₃ are N, the compound represented by Formula ET-1 may include a triazine group.

b1 to b3 may each independently be an integer of 0 to 10. L₁ to L3 may each independently be a direct linkage, a substituted or unsubstituted arylene group having 6 to 30 ring-forming carbon atoms, or a substituted or unsubstituted heteroarylene group having 2 to 30 ring-forming carbon atoms. When b1 to b3 are an integer of 2 or greater, respective L₁ to L3 may be each independently a substituted or unsubstituted arylene group having 6 to 30 ring-forming carbon atoms, or a substituted or unsubstituted heteroarylene group having 2 to 30 ring-forming carbon atoms.

Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, Ar₁ to Ar₃ may a substituted or unsubstituted phenyl group or a substituted or unsubstituted carbazole group. However, this is presented as an example, and the embodiment of the present disclosure is not limited thereto.

The compound represented by Formula ET-1 may be represented by any one selected from among compounds from Compound Group ET below. The light emitting element ED may include any one selected from among compounds of Compound Group ET below. In Compound Group ET below, D is a deuterium atom.

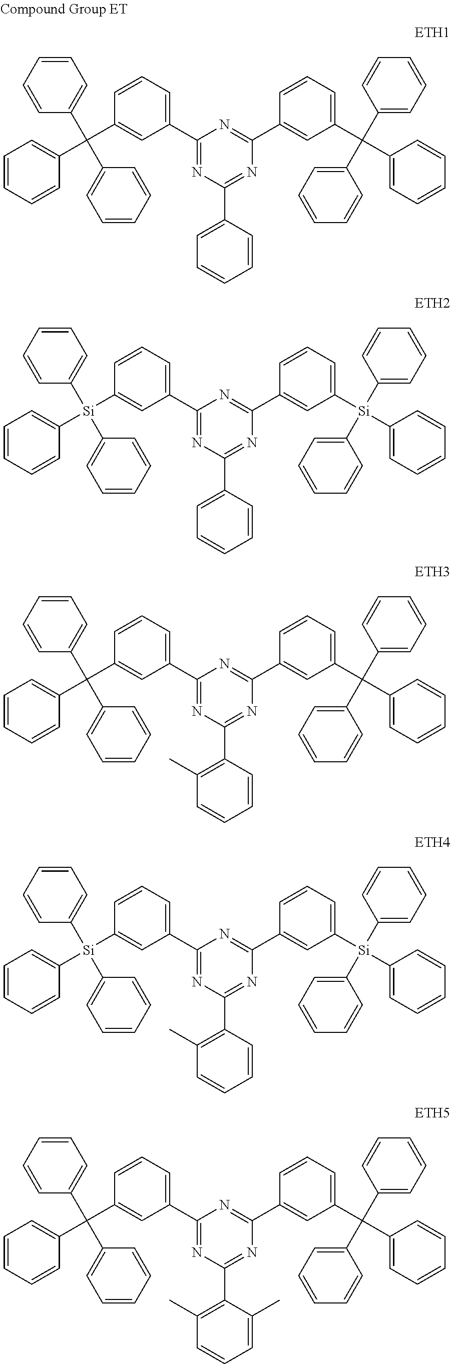

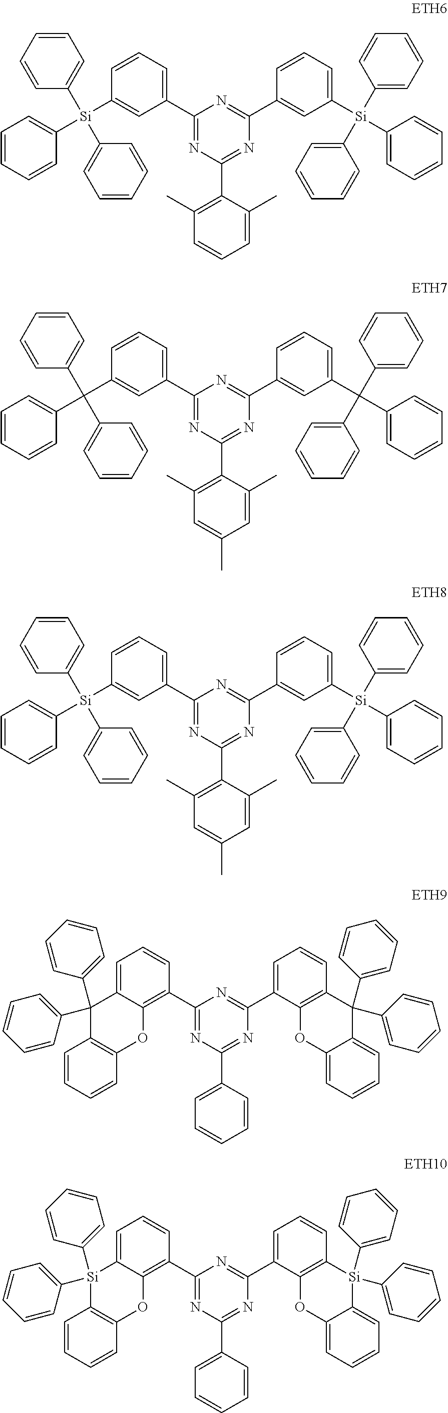

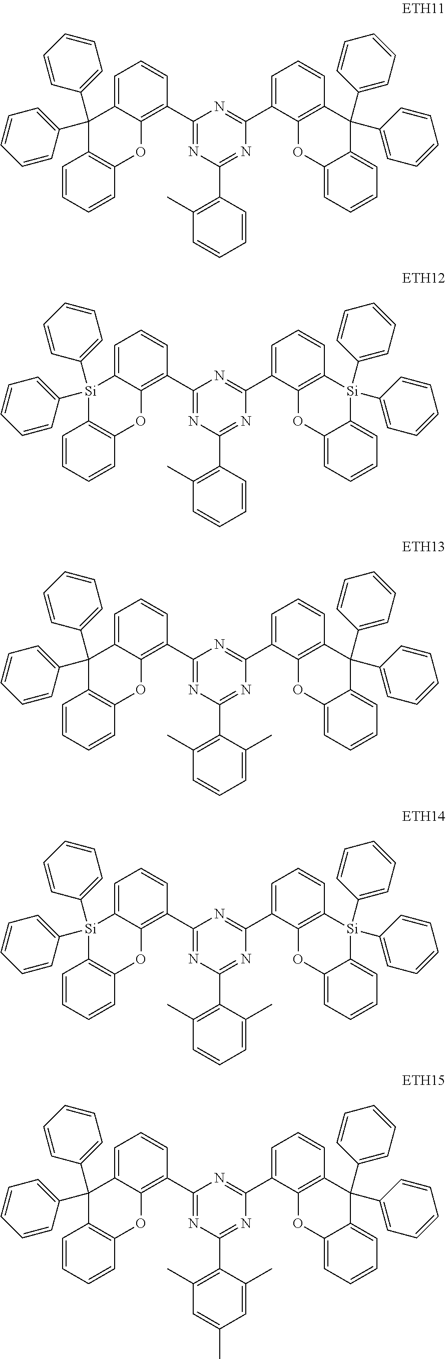

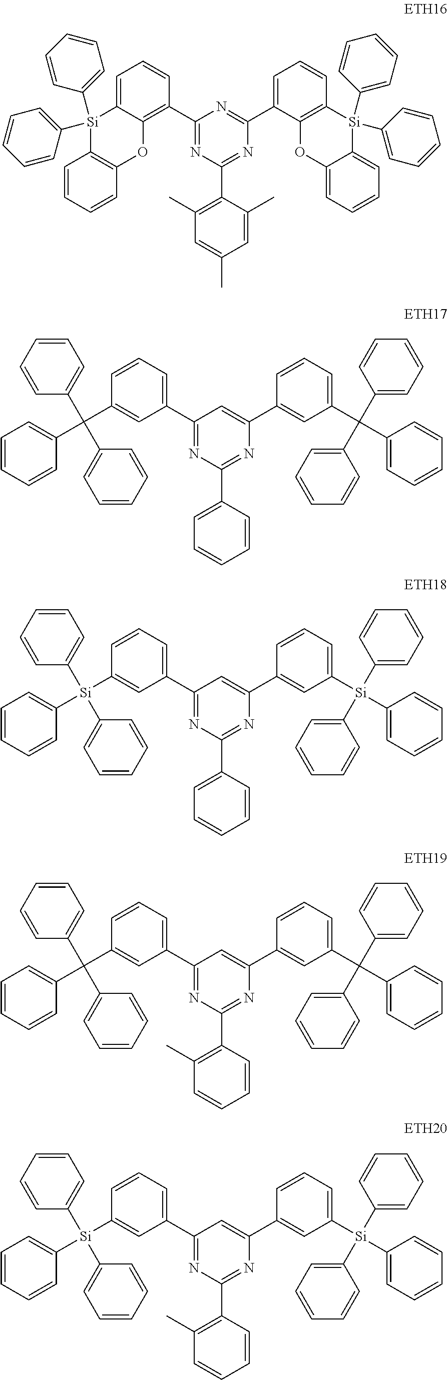

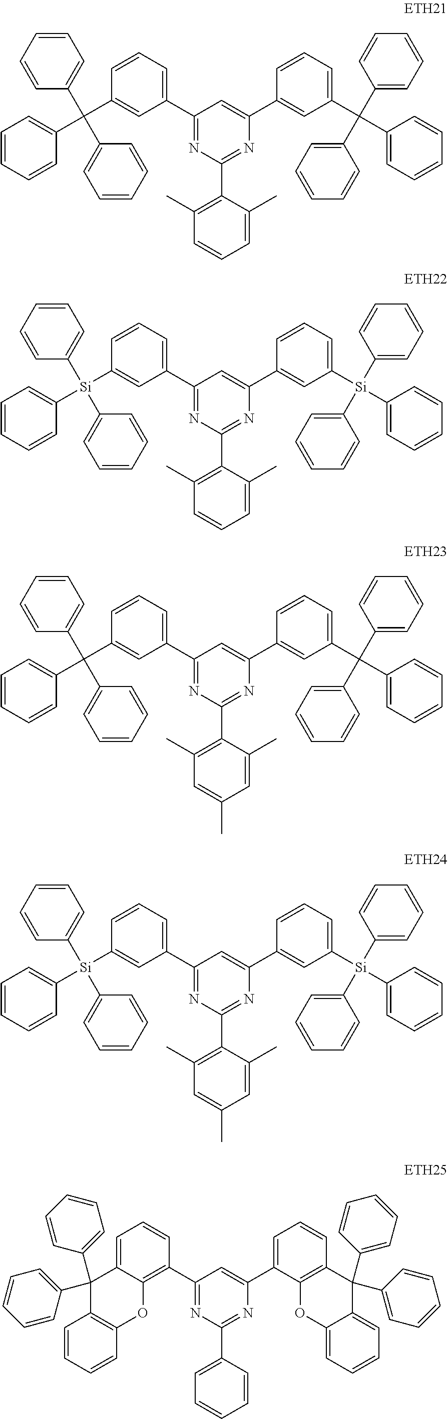

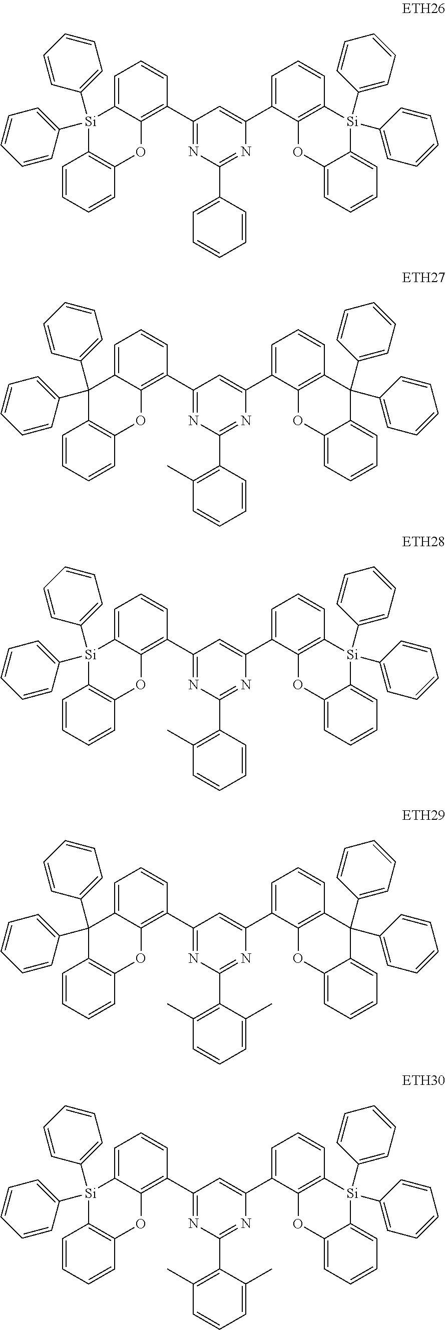

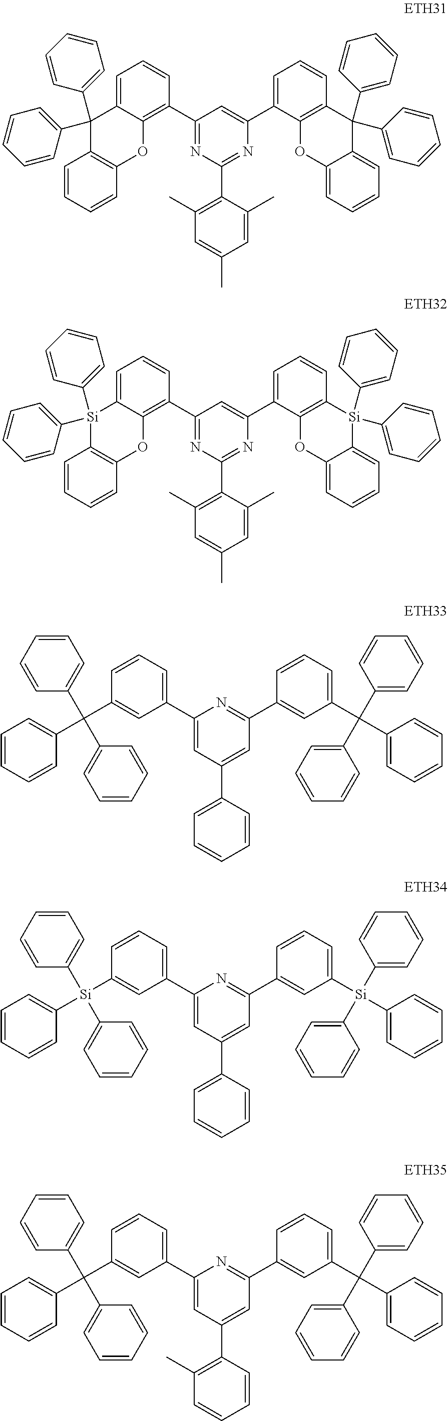

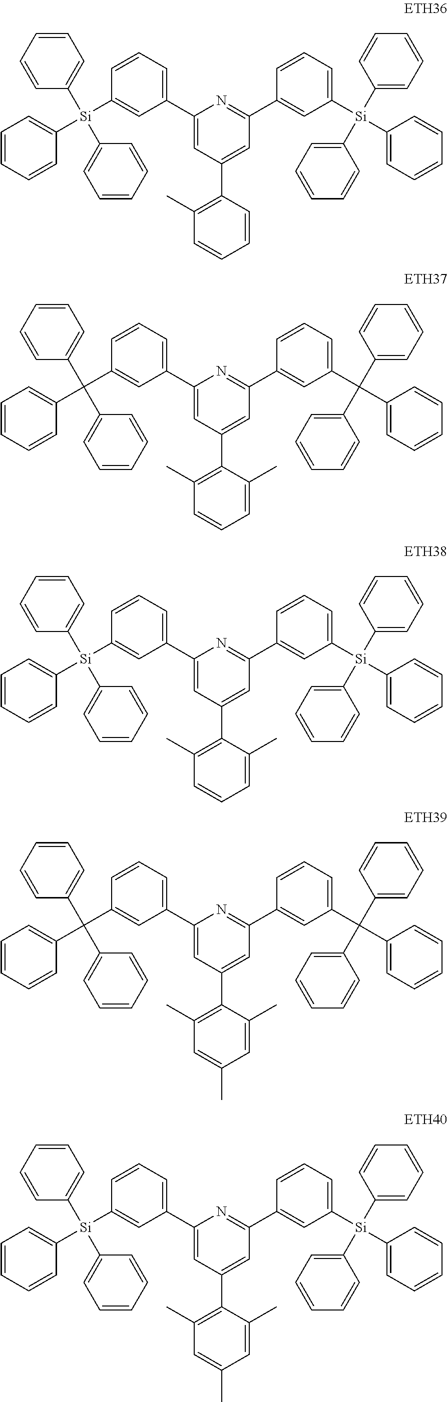

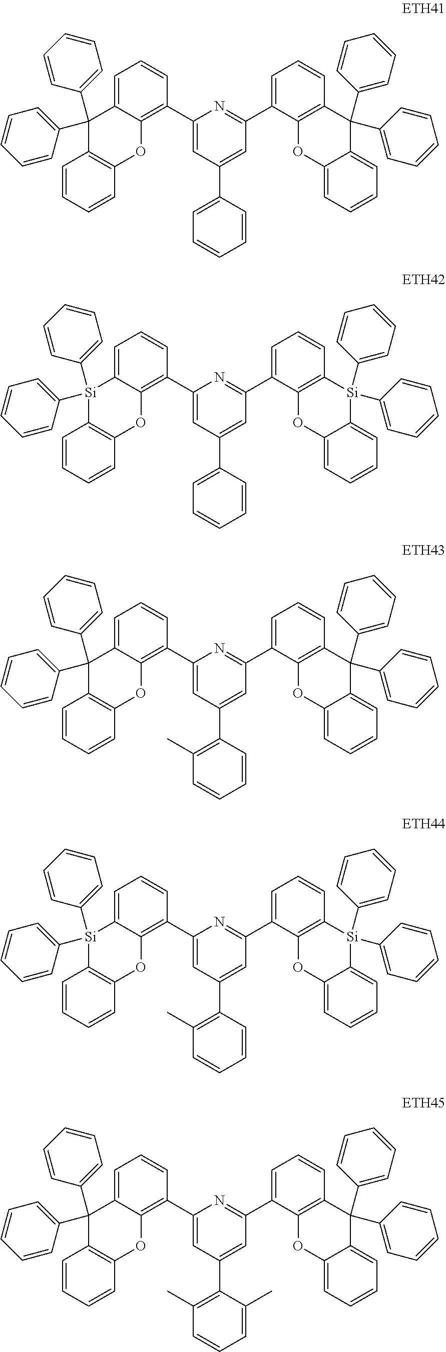

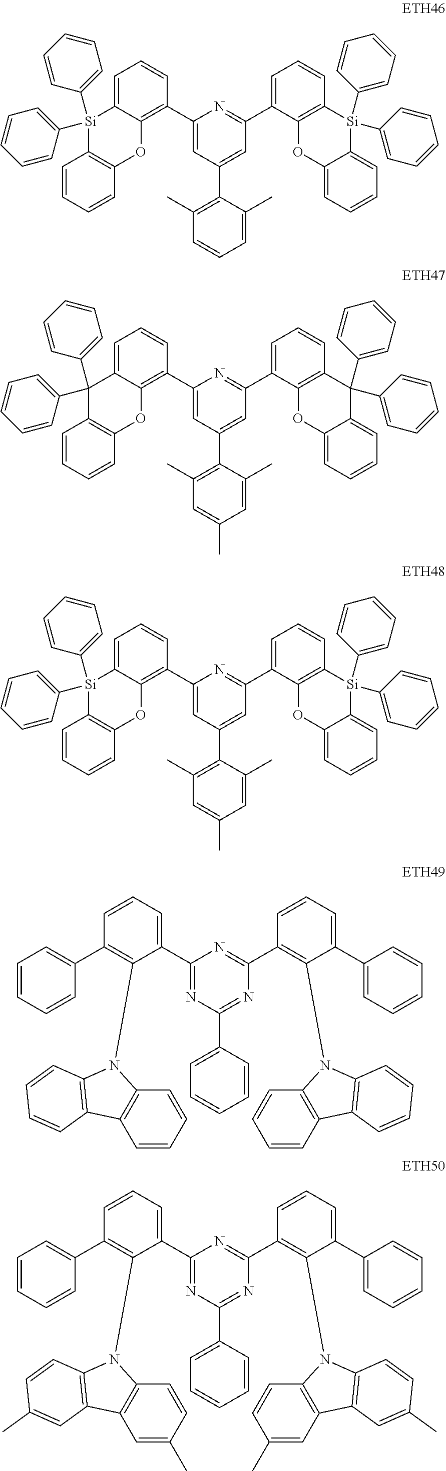

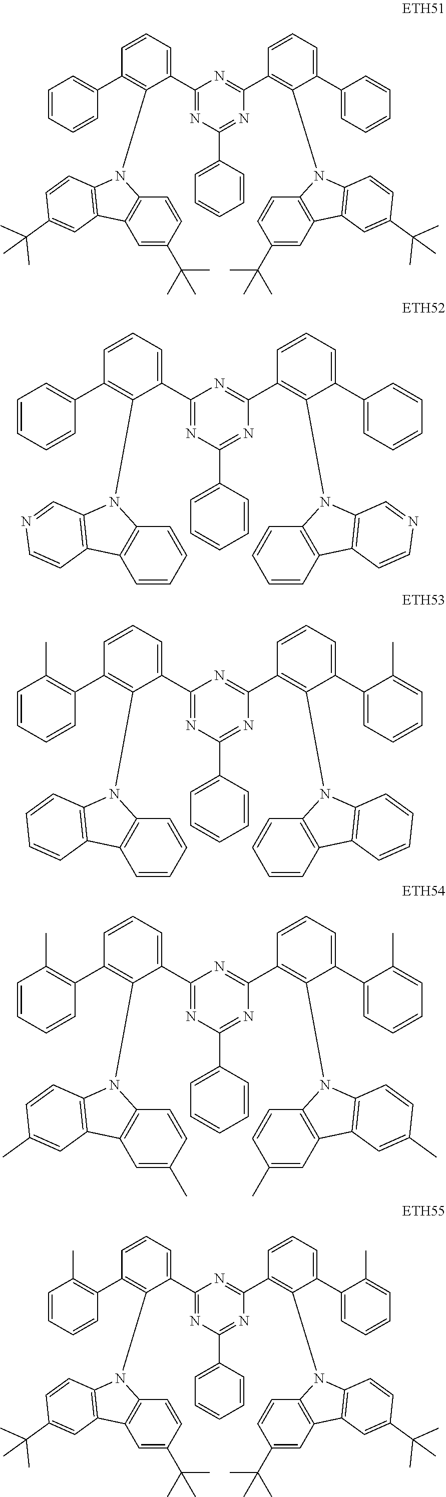

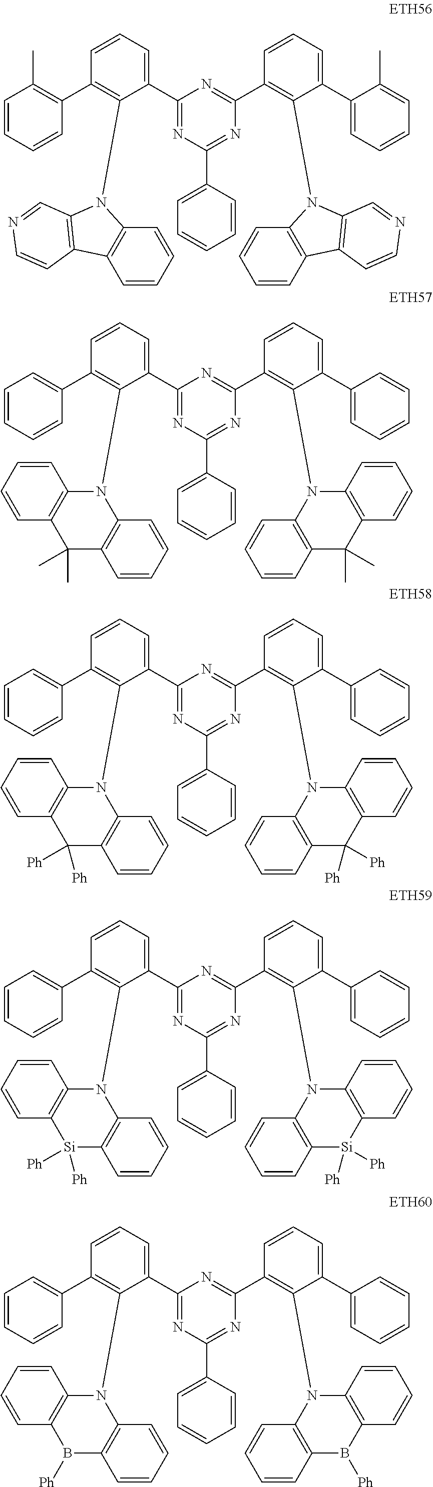

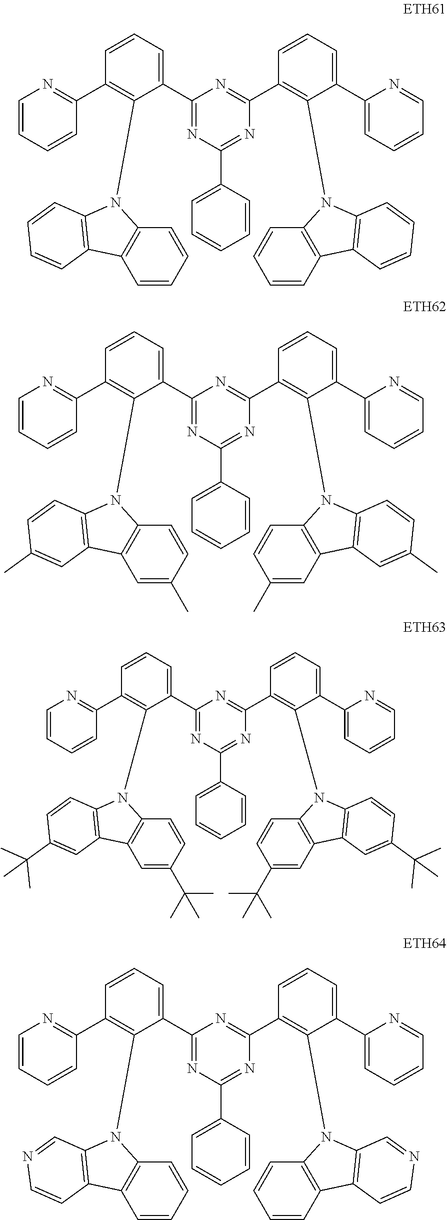

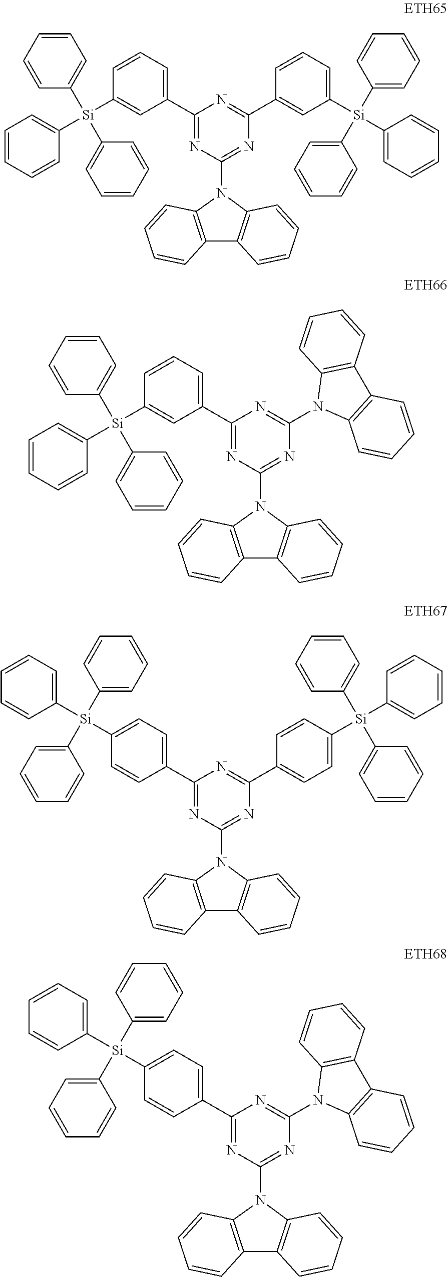

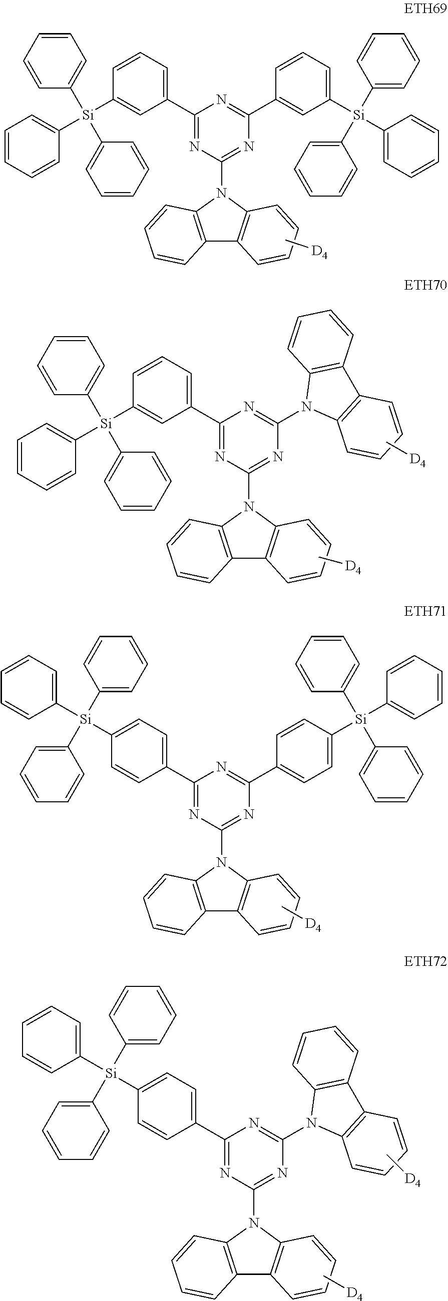

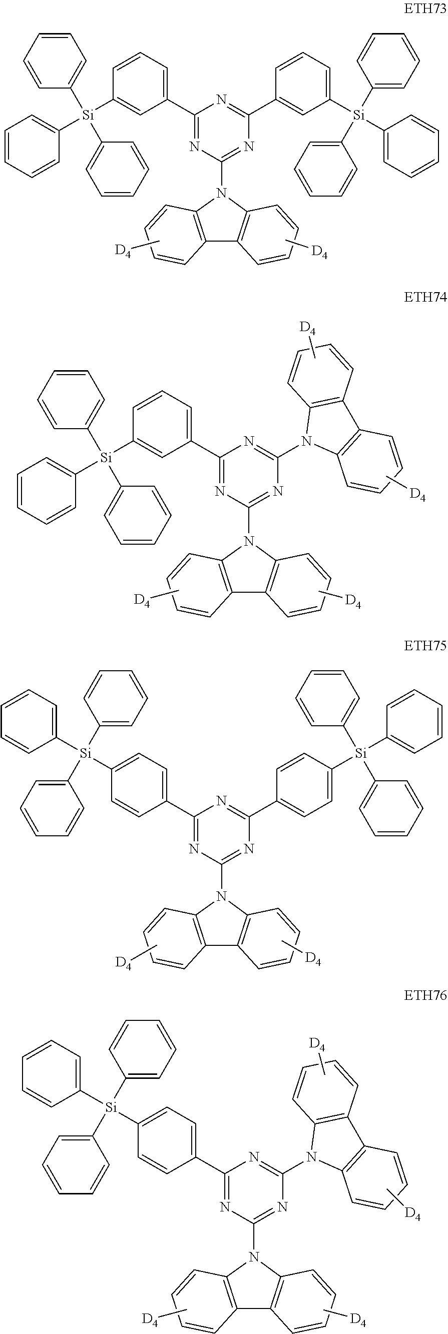

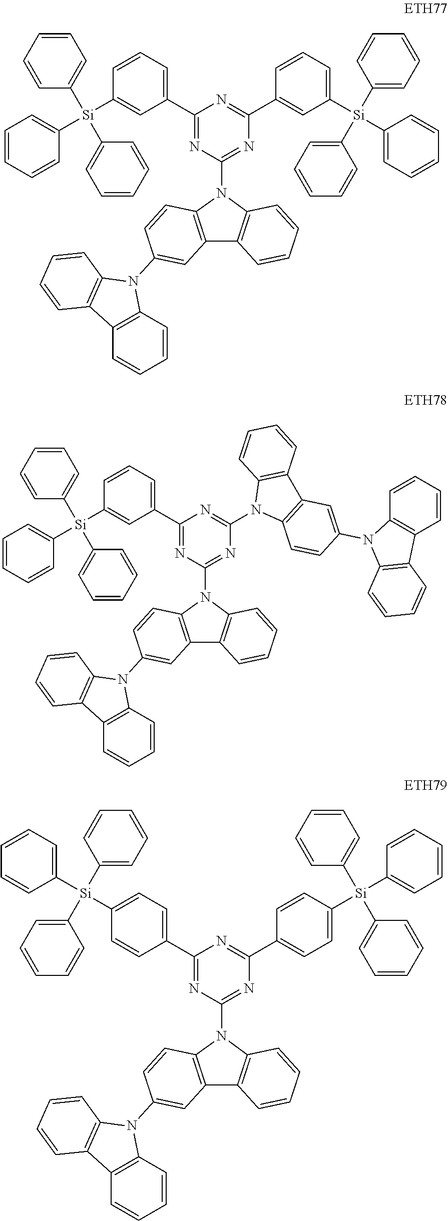 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5cc(-n6c7ccccc7c7ccccc76)ccc54)n3)c2)cc1 ; c1ccc([Si](c2ccccc2)(c2ccccc2)c2cccc(-c3nc(-n4c5ccccc5c5cc(-n6c7ccccc7c7ccccc76)ccc54)nc(-n4c5ccccc5c5cc(-n6c7ccccc7c7ccccc76)ccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5cc(-n6c7ccccc7c7ccccc76)ccc54)n3)cc2)cc1]

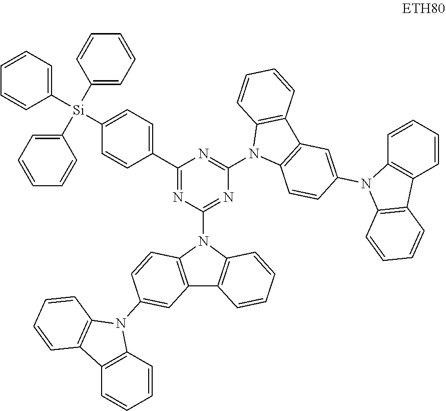 [SMILES: c1ccc([Si](c2ccccc2)(c2ccccc2)c2ccc(-c3nc(-n4c5ccccc5c5cc(-n6c7ccccc7c7ccccc76)ccc54)nc(-n4c5ccccc5c5cc(-n6c7ccccc7c7ccccc76)ccc54)n3)cc2)cc1]

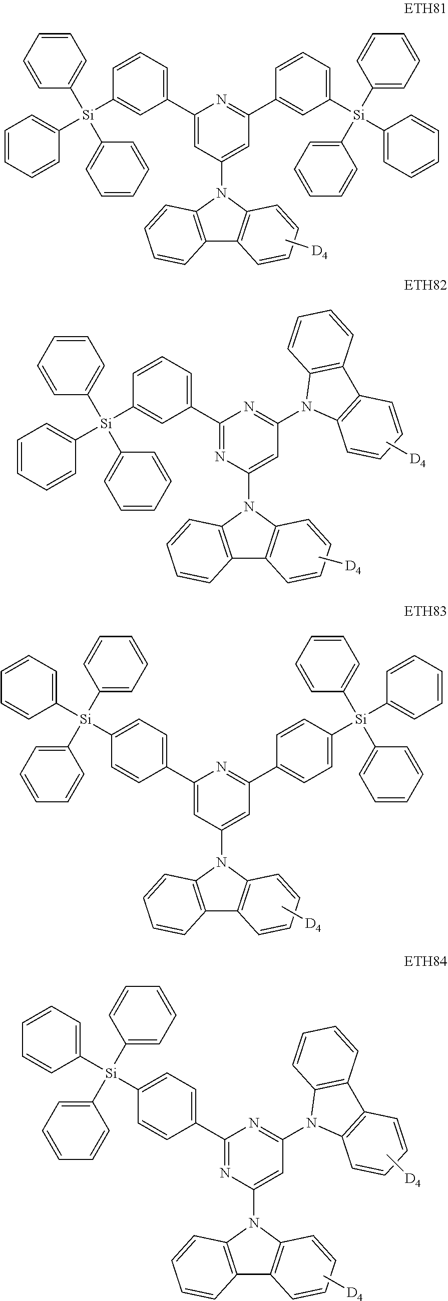

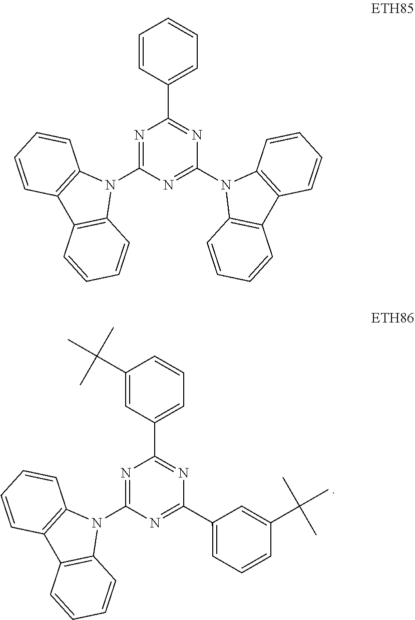 [SMILES: c1ccc(-c2nc(-n3c4ccccc4c4ccccc43)nc(-n3c4ccccc4c4ccccc43)n2)cc1 ; CC(C)(C)c1cccc(-c2nc(-c3cccc(C(C)(C)C)c3)nc(-n3c4ccccc4c4ccccc43)n2)c1]

In some embodiments, the emission layer EML may further include compounds that will be described below in addition to the heterocyclic compound of one or more embodiments. The emission layer EML may include an anthracene derivative, a pyrene derivative, a fluoranthene derivative, a chrysene derivative, a dihydrobenzanthracene derivative, and/or a triphenylene derivative. For example, the emission layer EML may include an anthracene derivative and/or a pyrene derivative.

The emission layer EML may include a compound represented by Formula E-1 below. The compound represented by Formula E-1 below may be used as a fluorescent host material.

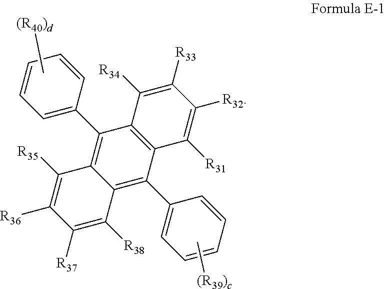

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, a substituted or unsubstituted heteroaryl group having 2 to 30 ring-forming carbon atoms, and/or bonded to an adjacent group to form a ring. In some embodiments, R₃₁ to R₄₀ may be bonded to an adjacent group to form a saturated hydrocarbon ring, an unsaturated hydrocarbon ring, a saturated heterocycle, or an unsaturated heterocycle.

In Formula E-1, c and d may each independently be an integer of 0 to 5. When c is an integer of 2 or greater, a plurality of R₃₉'s may all be the same or at least one may be different from the others. When d is an integer of 2 or greater, a plurality of R₄₀'s may all be the same or at least one may be different from the others. Formula E-1 may be represented by any one selected from among compounds E1 to E19 below.

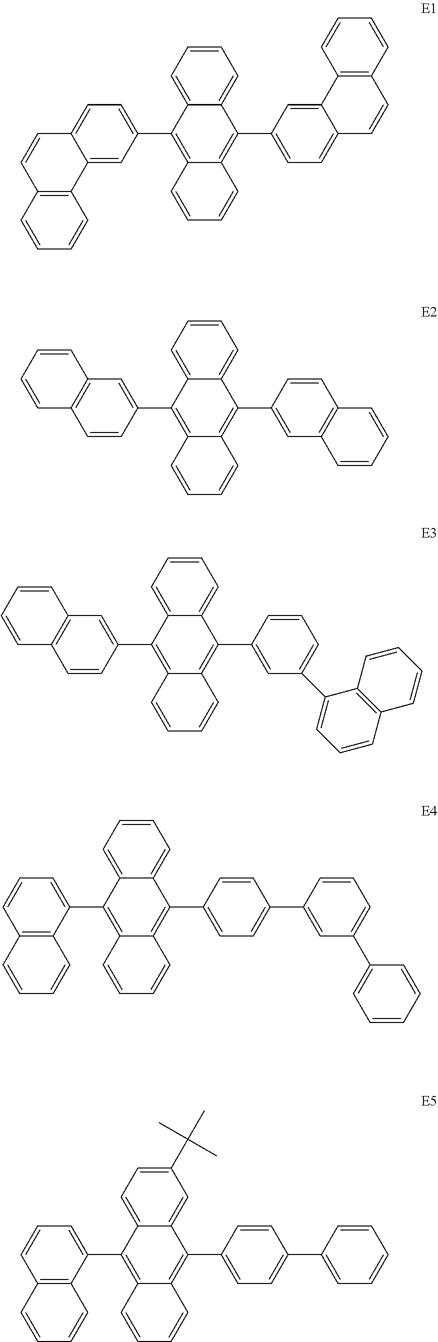

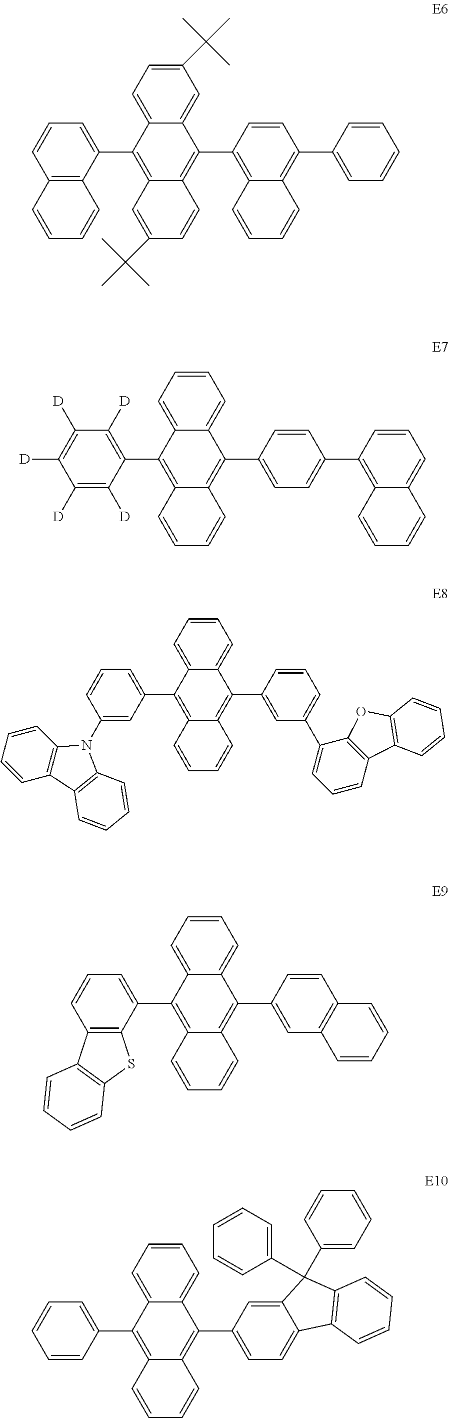

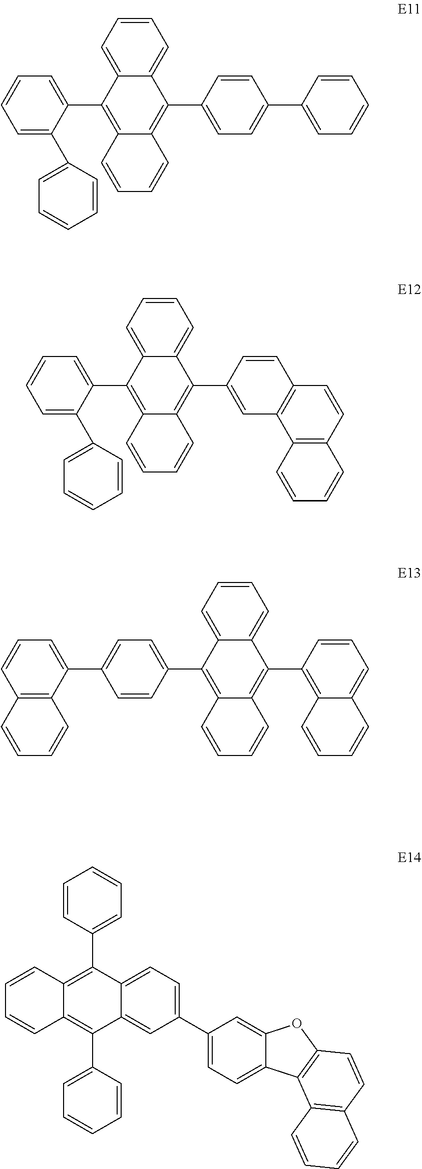

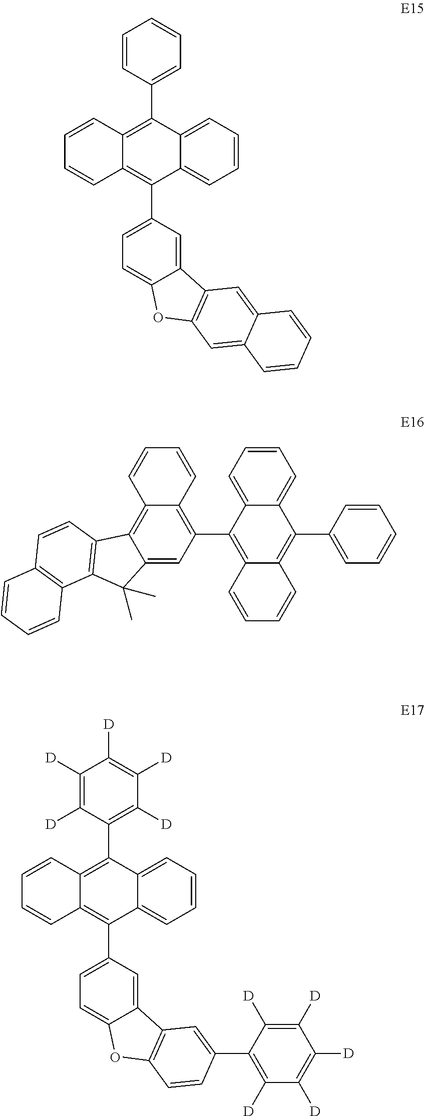

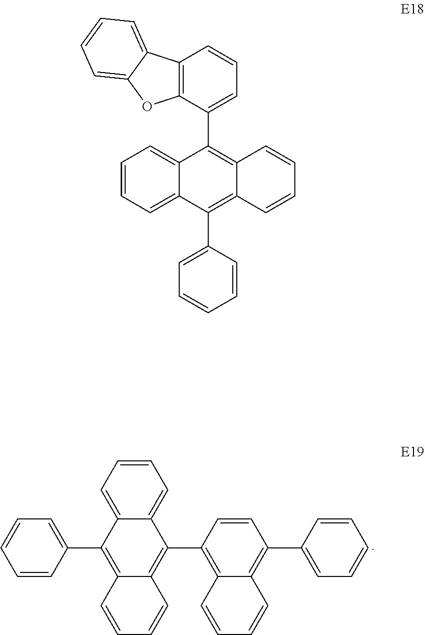

In one or more embodiments, the emission layer EML may include a compound represented by Formula E-2a or Formula E-2b below. The compound represented by Formula E-2a or Formula E-2b may be used as a phosphorescent host material

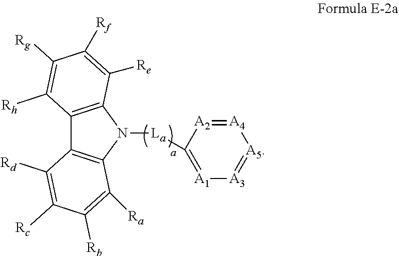

In Formula E-2a, a may be an integer of 0 to 10, and La may be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a is an integer of 2 or greater, a plurality of La's may each independently be a substituted or unsubstituted arylene group having 6 to 30 ring-forming carbon atoms, or a substituted or unsubstituted heteroarylene group having 2 to 30 ring-forming carbon atoms.

In some embodiments, in Formula E-2a, A₁ to A5 may each independently be N or CRᵢ. Rₐ to R₁ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, a substituted or unsubstituted heteroaryl group having 2 to 30 ring-forming carbon atoms, and/or bonded to an adjacent group to form a ring. Rₐ to Rᵢ may be bonded to an adjacent group to form a hydrocarbon ring or a heterocycle containing N, O, S, and/or the like as a ring-forming atom.

In some embodiments, in Formula E-2a, two or three selected from A₁ to A5 may be N, and the rest may be CRᵢ.

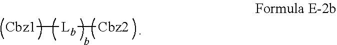

In Formula E-2b, Cbz1 and Cbz2 may each independently be an unsubstituted carbazole group or an aryl-substituted carbazole group having 6 to 30 ring-forming carbon atoms. L_{b} may be a direct linkage, a substituted or unsubstituted arylene group having 6 to 30 ring-forming carbon atoms, or a substituted or unsubstituted heteroarylene group having 2 to 30 ring-forming carbon atoms, b may be an integer of 0 to 10, and when b is an integer of 2 or greater, a plurality of L_{b}'s may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one selected from among compounds from Compound Group E-2 below. However, the compounds listed in Compound Group E-2 below are presented as an example, and the compound represented by Formula E-2a or Formula E-2b is not limited to those listed in Compound Group E-2 below.

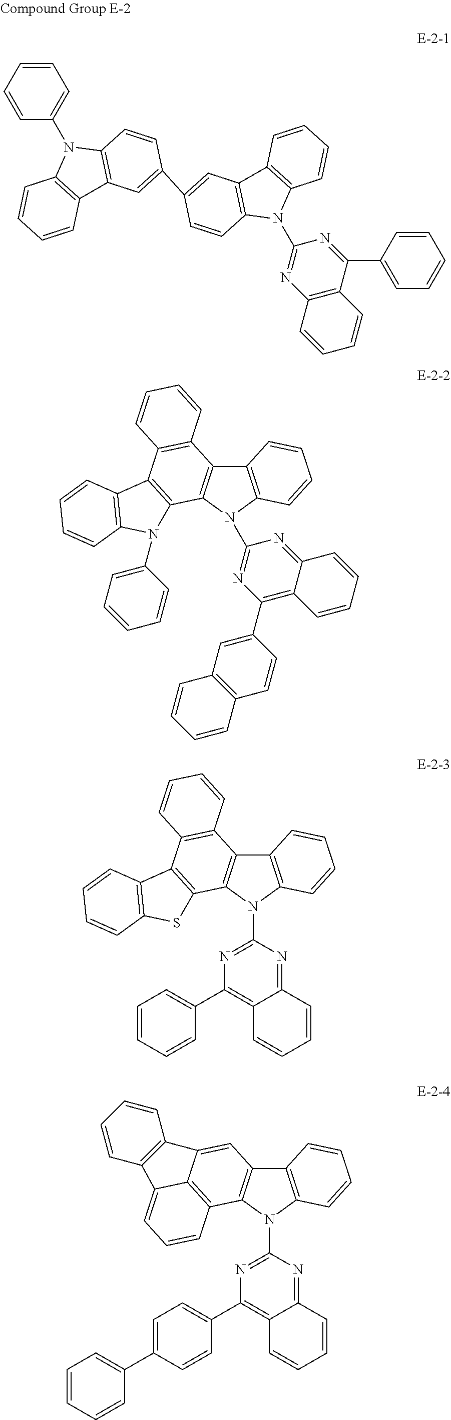

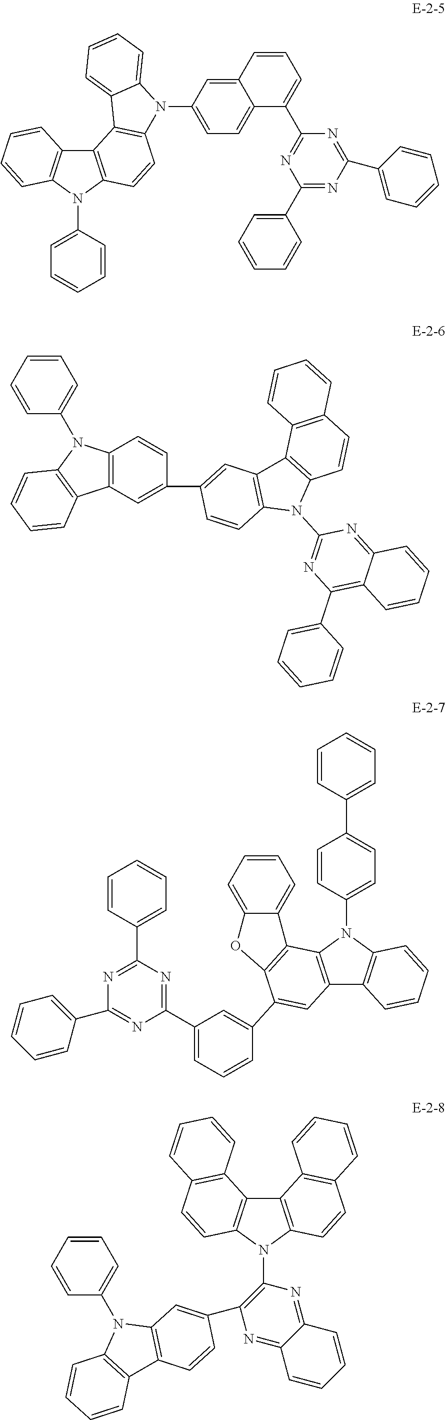

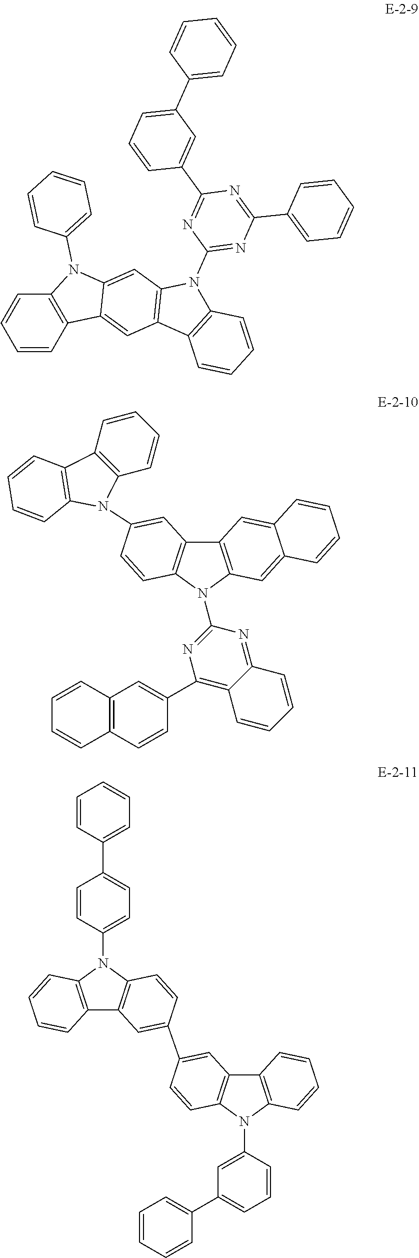

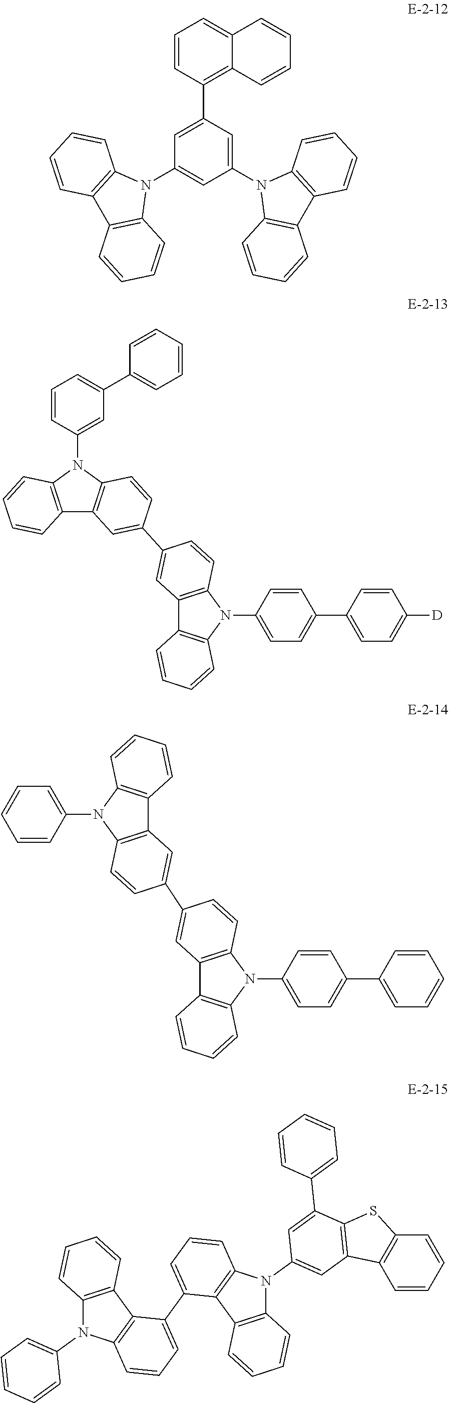 [SMILES: c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

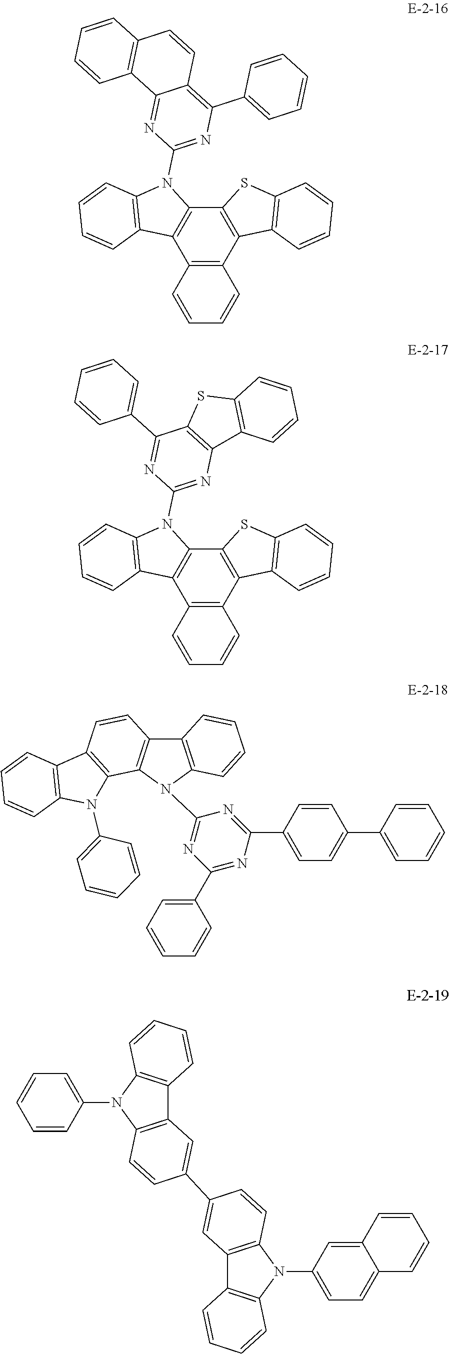 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1]

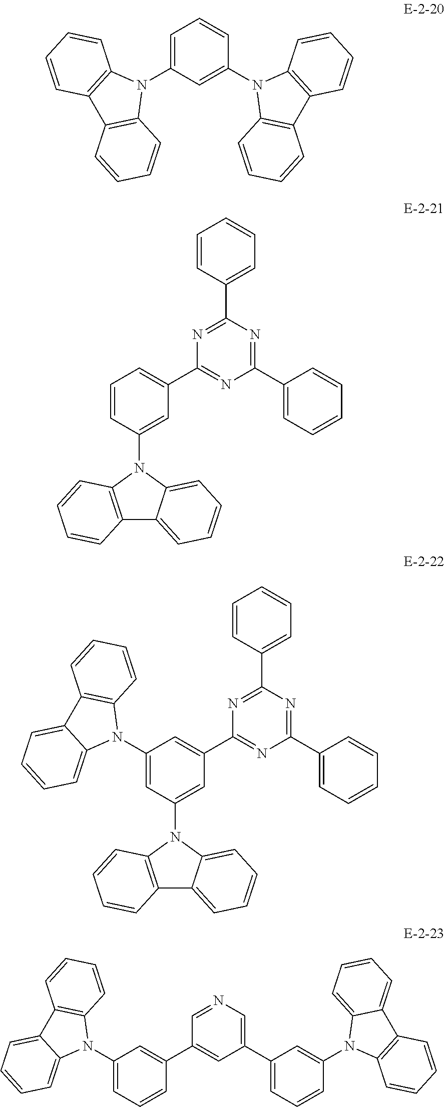

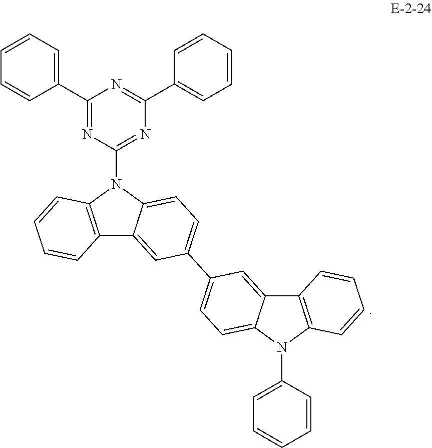 [SMILES: c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a suitable host material. For example, the emission layer EML may include, as a host material, at least one selected from among bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzofuran (PPF), 4,4′,4″-tris(carbazol-9-yl)-triphenylamine (TCTA), and 1,3,5-tris(1-phenyl-1H-benzo[d]imidazol-2-yl)benzene (TPBi). However, the embodiment of the present disclosure is not limited thereto, and for example, tris(8-hydroxyquinolino)aluminum (Alq3), 9,10-di(naphthalene-2-yl)anthracene (ADN), 3-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetrasiloxane (DPSiO₄), and/or the like may be used as a host material.

The emission layer EML may include a compound represented by Formula M-a or Formula M-b below. The compound represented by Formula M-a or Formula M-b below may be used as a phosphorescent dopant material.

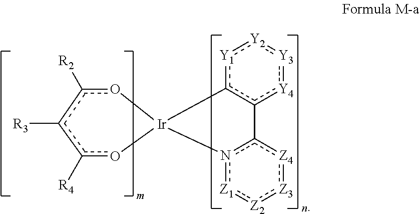

In Formula M-a above, Y₁ to Y₄, and Z₁ to Z₄ may each independently be CR₁ or N, and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, a substituted or unsubstituted heteroaryl group having 2 to 30 ring-forming carbon atoms, and/or bonded to an adjacent group to form a ring. In Formula M-a, m is 0 or 1, and n is 2 or 3. In Formula M-a, when m is 0, n is 3, and when m is 1, n is 2.

The compound represented by Formula M-a may be represented by any one selected from among compounds M-a1 to M-a25 below. However, the compounds M-a1 to M-a25 below are presented as an example, and the compound represented by Formula M-a is not limited to those represented by the compounds M-a1 to M-a25 below.

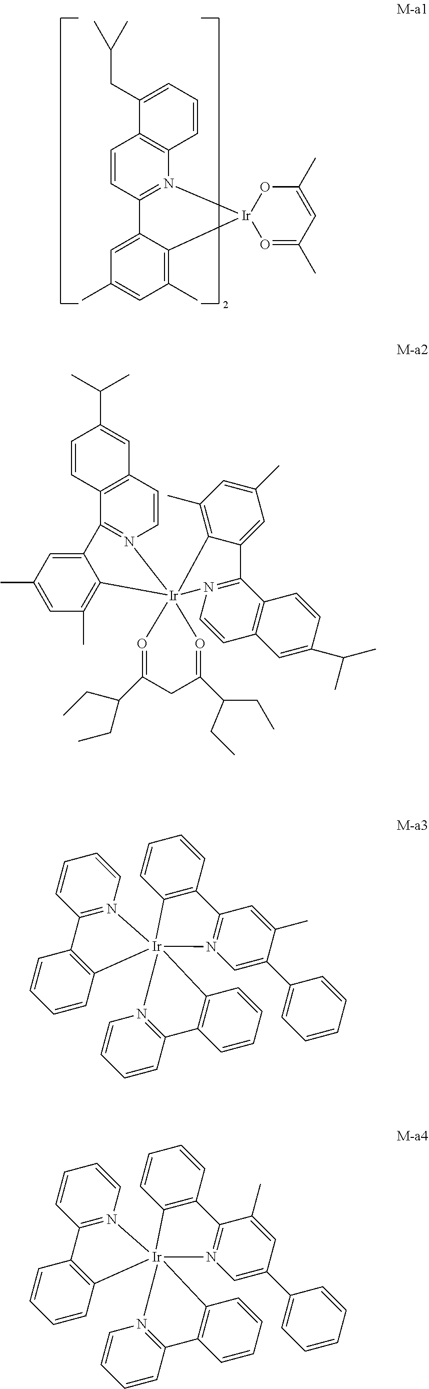

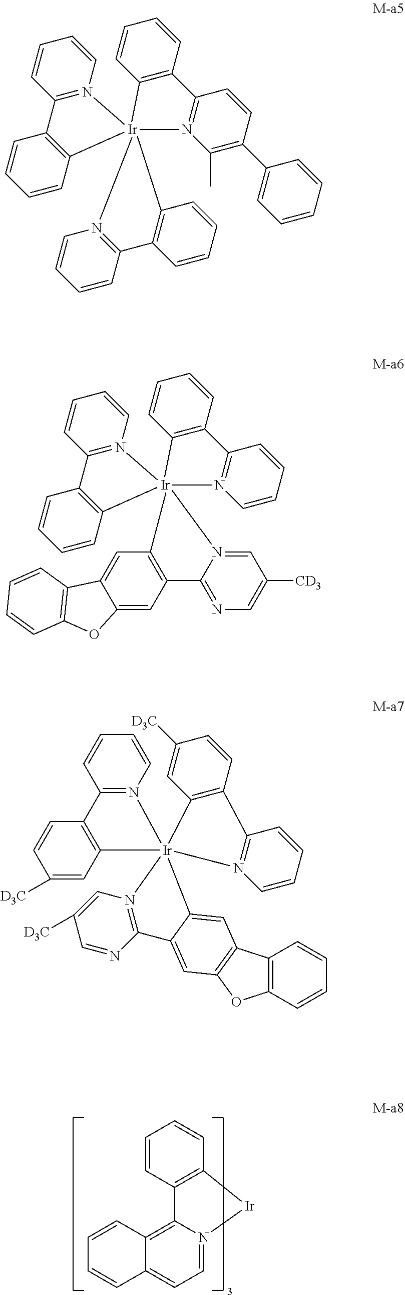

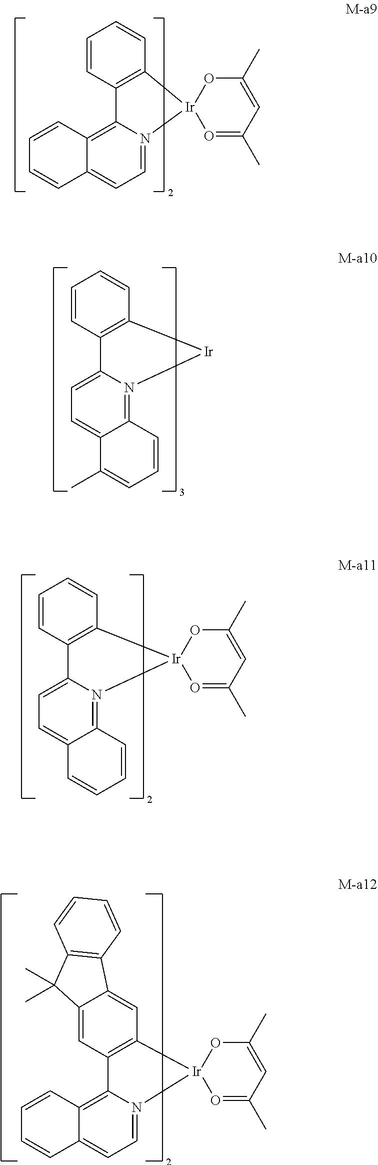

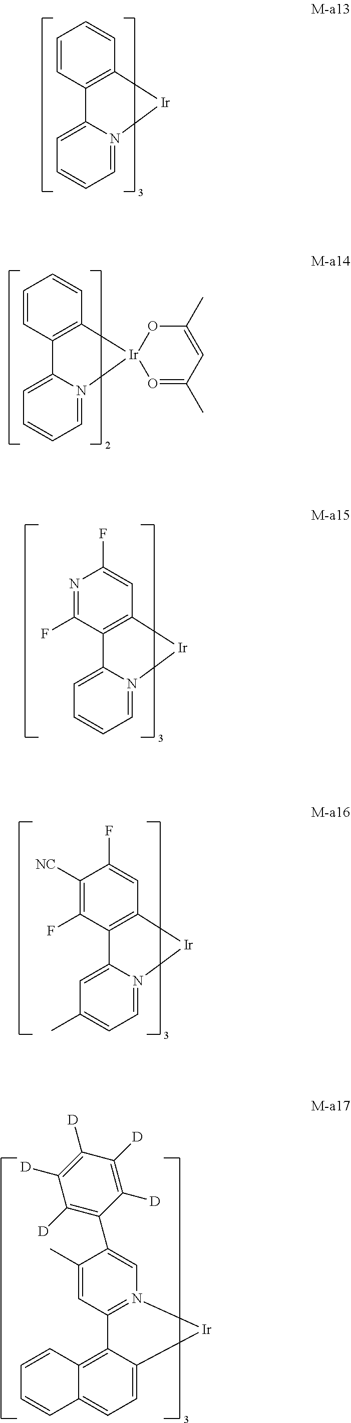

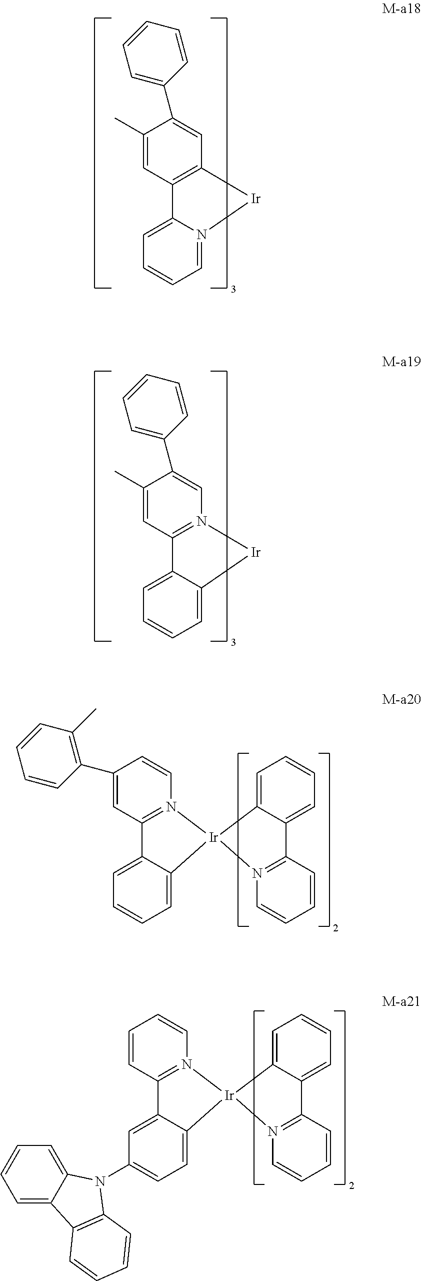

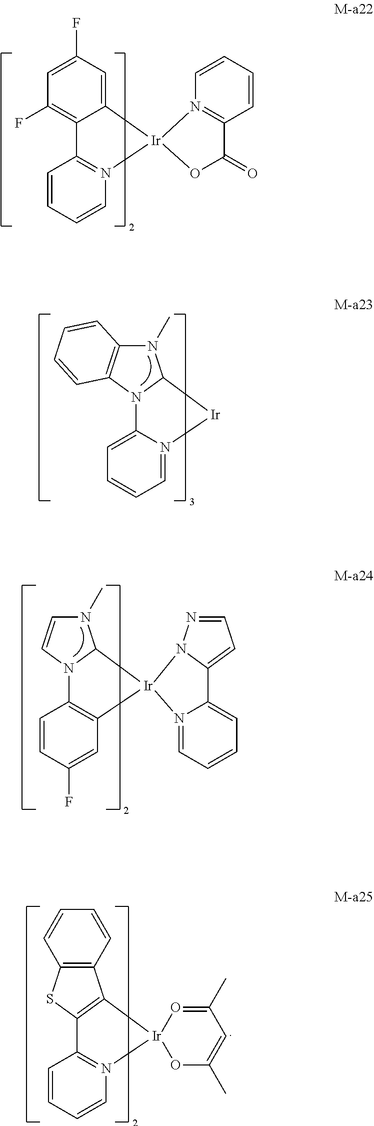

The compounds M-a1 and M-a2 may be used as a red dopant material. The compounds M-a3 to M-a7 may be used as a green dopant material.

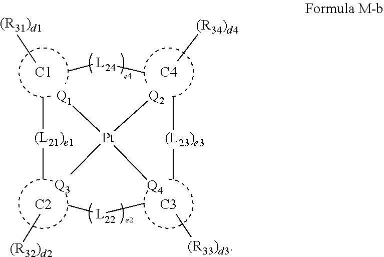

In Formula M-b, Q1 to 04 may each independently be C or N, and C1 to 04 may each independently be a substituted or unsubstituted hydrocarbon ring having 5 to 30 ring-forming carbon atoms, or a substituted or unsubstituted heterocycle having 2 to 30 ring-forming carbon atoms. L21 to L24 may each independently be a direct linkage,

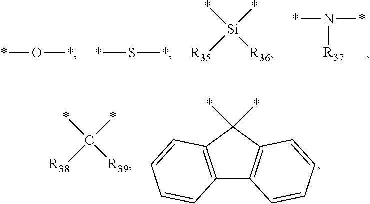

substituted or unsubstituted divalent alkyl group having 1 to 20 carbon atoms, a substituted or unsubstituted arylene group having 6 to 30 ring-forming carbon atoms, or substituted or unsubstituted heteroarylene group having 2 to 30 ring-forming carbon atoms, and e1 to e4 may each independently be 0 or 1.

In Formula M-b, R₃₁ to R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring. d1 to d4 may each independently be an integer of 0 to 4.

A compound represented by Formula M-b may be used as a blue phosphorescent dopant or a green phosphorescent dopant. The compound represented by Formula M-b may be represented by any one selected from among compounds from Compound Group AD below. However, the compounds below are presented as an example, and the compound represented by Formula M-b is not limited to those represented by the compounds below.

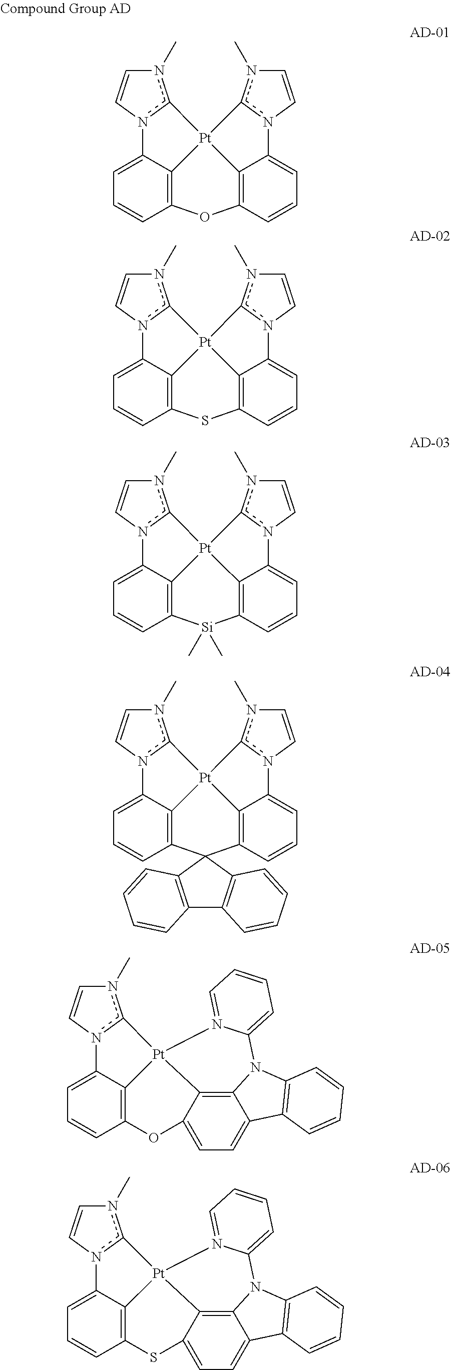

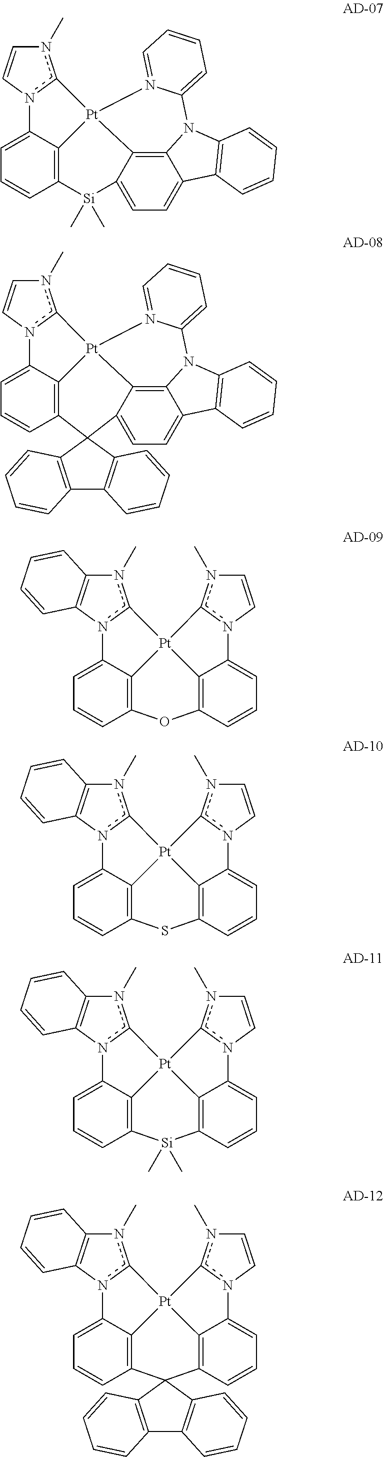

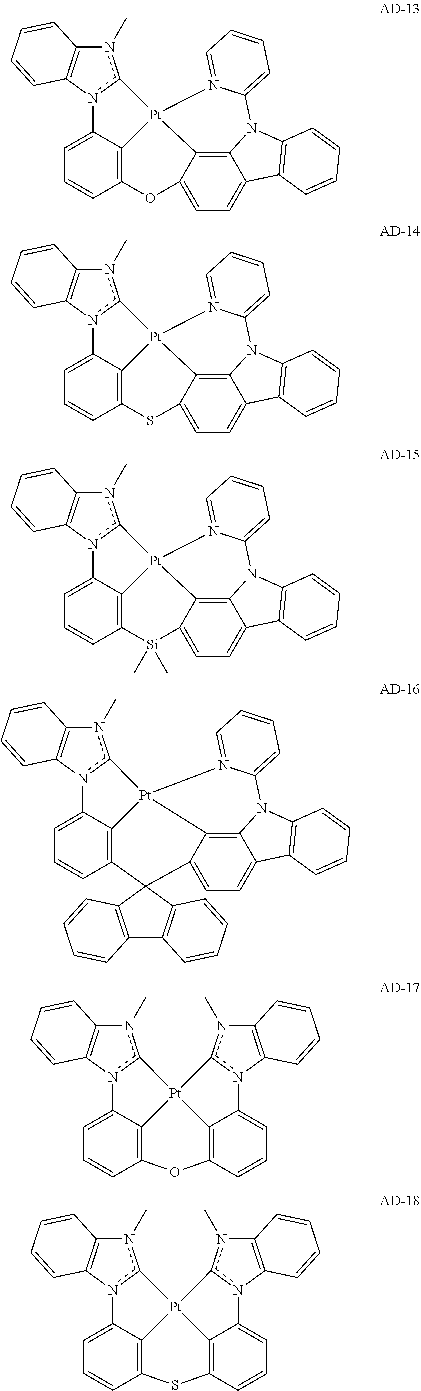

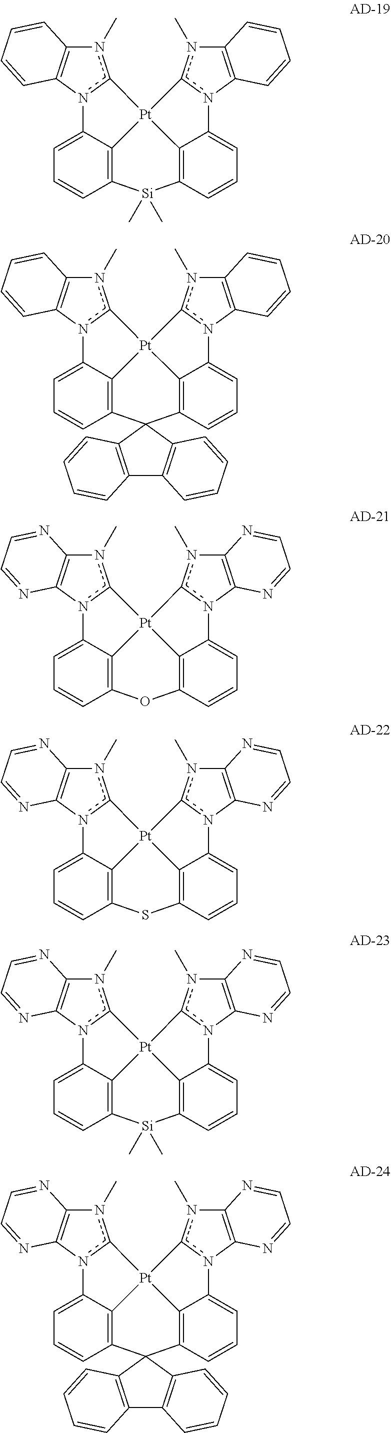

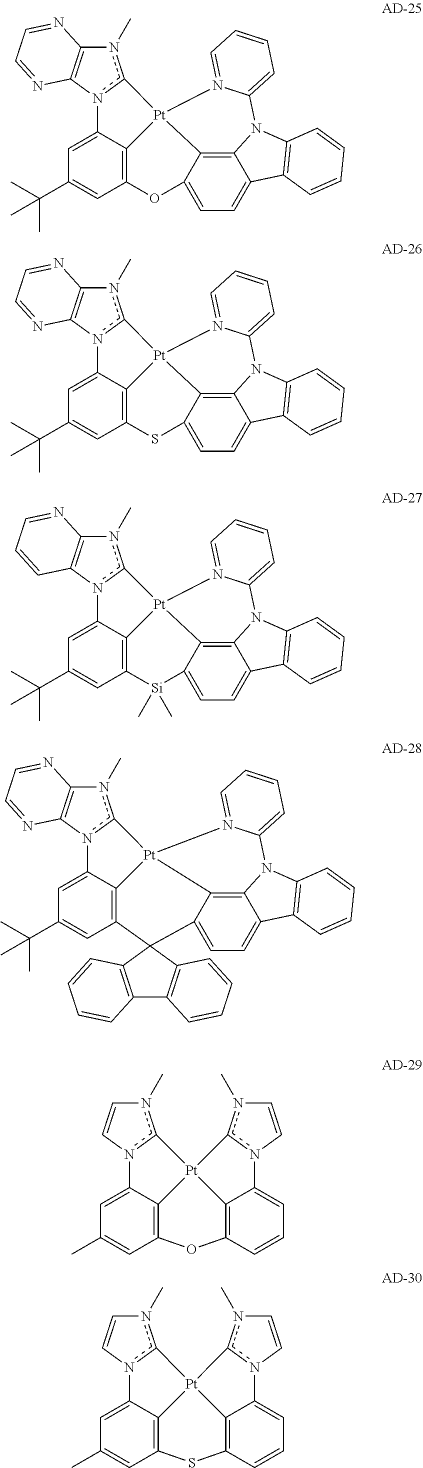

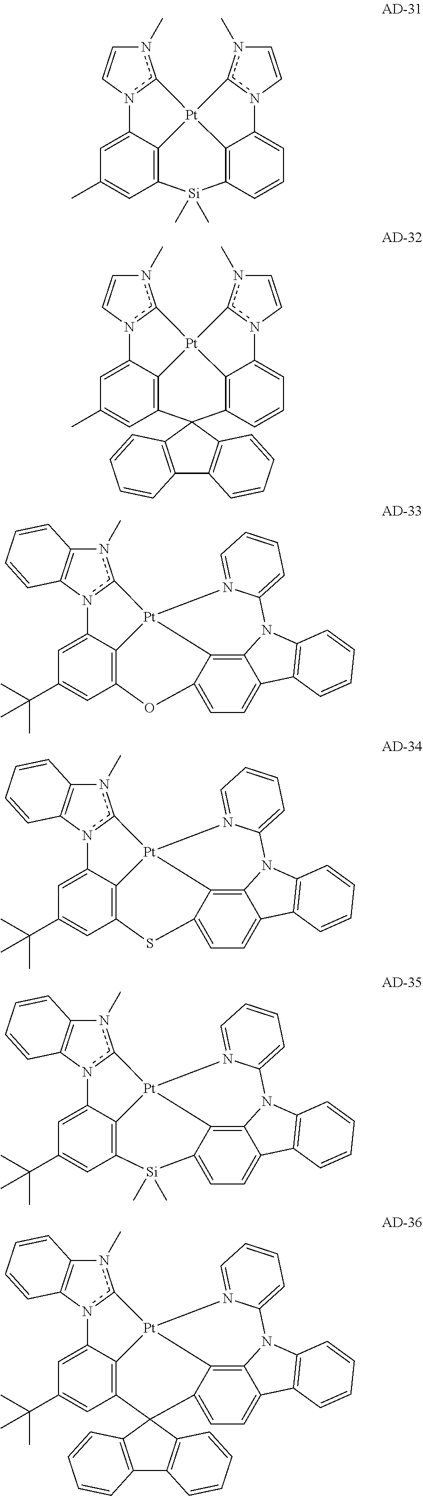

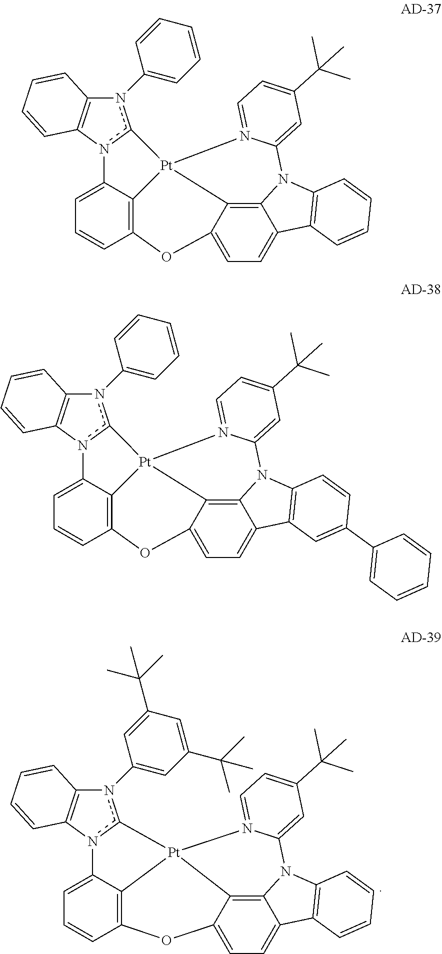

The emission layer EML may include a compound represented by any one selected from among Formulae F-a to F-c below. The compounds represented by Formulae F-a to F-c below may be used as a fluorescence dopant material.

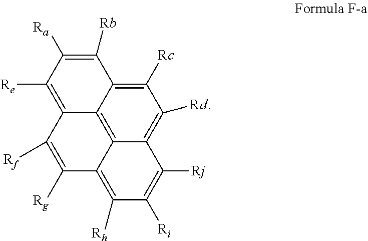

In Formula F-a above, two selected from Rₐ to Rⱼ may each independently be substituted with *—NAr₁Ar₂. The others selected from among Rₐ to Rⱼ which are not substituted with *—NAr₁Ar₂ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In *—NAr₁Ar₂, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, at least one of Ar₁ or Ar₂ may be a heteroaryl group containing O or S as a ring-forming atom.

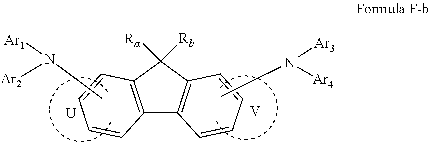

In Formula F-b above, Rₐ and R_{b} may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring. Ar₁ to Ar₄ may each independently be a substituted or unsubstituted aryl group having 6 to 30 ring-forming carbon atoms, or a substituted or unsubstituted heteroaryl group having 2 to 30 ring-forming carbon atoms.

In Formula F-b, U and V may each independently be a substituted or unsubstituted hydrocarbon ring having 5 to 30 ring-forming carbon atoms, or a substituted or unsubstituted heterocycle having 2 to 30 ring-forming carbon atoms. In Formula F-b, the number of rings represented by U and V may each independently be 0 or 1.

For example, in Formula F-b, when the number of U or V is 1, one ring forms a fused ring in a portion indicated by U or V, and when the number of U or V is 0, it means that no ring indicated by U or V is present. When the number of U is 0 and the number of V is 1, or when the number of U is 1 and the number of V is 0, a fused ring having a fluorene core of Formula F-b may be a cyclic compound having four rings. When both U and V are 0, the fused ring having a fluorene core of Formula F-b may be a cyclic compound having three rings. When both U and V are 1, the fused ring having a fluorene core of Formula F-b may be a cyclic compound having five rings.

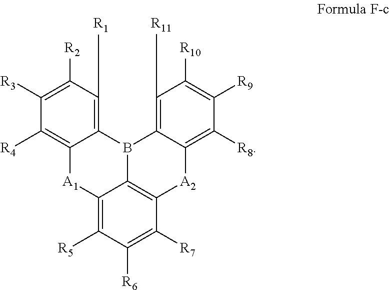

In Formula F-c, A₁ and A₂ may each independently be O, S, Se, or NRₘ, and Rm may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boron group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring.

In Formula F-c, A₁ and A₂ may each independently be bonded to substituent(s) of neighboring rings to form a fused ring. For example, when A₁ and A₂ are each independently NRₘ, A₁ may be bonded to R₄ or R₅ to form a ring. In some embodiments, A₂ may be bonded to R₇ or R₈ to form a ring.

The emission layer EML may include, as a suitable dopant material, styryl derivatives (e.g., 1,4-bis[2-(3-N-ethylcarbazolyl)vinyl]benzene (BCzVB), 4-(di-p-tolylamino)-4′-[(di-p-tolylamino)styryl]stilbene (DPAVB), and/or N-(4-((E)-2-(6-((E)-4-(diphenylamino)styryl)naphthalen-2-yl)vinyl)phenyl)-N-phenylbenzenamine (N-BDAVBi)), perylene and/or derivatives thereof (e.g., 2,5,8,11-tetra-t-butylperylene (TBP)), pyrene and/or derivatives thereof (e.g., 1,1′-dipyrene, 1,4-dipyrenylbenzene, 1,4-bis(N,N-diphenylamino)pyrene), and/or the like.

The emission layer EML may include a suitable phosphorescent dopant material. For example, as a phosphorescent dopant, a metal complex including iridium (Ir), platinum (Pt), osmium (Os), gold (Au), titanium (Ti), zirconium (Zr), hafnium (Hf), europium (Eu), and terbium (Tb), and/or thulium (Tm) may be used. For example, iridium(III) bis(4,6-difluorophenylpyridinato-N,C2′)picolinate (Firpic), bis(2,4-difluorophenylpyridinato)-tetrakis(1-pyrazolyl)borate iridium(III) (Fir6), platinum octaethyl porphyrin (PtOEP), and/or the like may be used as a phosphorescent dopant. However, the embodiment of the present disclosure is not limited thereto.

The emission layer EML may include a quantum dot material. The core of a quantum dot may be selected from a Group II-VI compound, a Group I-II-VI compound, a Group II-IV-VI compound, a Group I-II-IV-VI compound, a Group III-VI compound, a Group I-III-VI compound, a Group III-V element, a Group III-II-V compound, a Group II-IV-V compound, a Group IV-VI compound, a Group IV element, a Group IV compound, and a combination thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and mixtures thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and mixtures thereof.

In some embodiments, the Group II-VI compound may further include a Group I metal and/or a Group IV element. The Group I-II-VI compound may be selected from CuSnS and/or CuZnS, and the Group II-IV-VI compound may be selected from ZnSnS and/or the like. The Group I-II-IV-VI compound may be selected from quaternary compounds selected from the group consisting of Cu₂ZnSnS2, Cu₂ZnSnS₄, Cu₂ZnSnSe₄, Ag₂ZnSnS₂, and mixtures thereof.

The Group III-VI compound may include a binary compound such as In₂S₃ and/or In₂Se₃; a ternary compound such as InGaS₃ and/or InGaSe₃; or any combination thereof.

The Group I-III-VI compound may be selected from a ternary compound selected from the group consisting of AgInS, AgInS₂, CulnS, CuInS₂, AgGaS₂, CuGaS₂ CuGaO₂, AgGaO₂, AgAlO₂, and mixtures thereof, and a quaternary compound such as AgInGaS₂ and/or CuInGaS₂.

The Group III-V compound may be selected from the group consisting of a binary compound selected from the group consisting of GaN, GaP, GaAs, GaSb, AlN, AIP, AIAs, AISb, InN, InP, InAs, InSb, and mixtures thereof; a ternary compound selected from the group consisting of GaNP, GaNAs, GaNSb, GaPAs, GaPSb, AlNP, AINAs, AlNSb, AIPAs, AIPSb, InGaP, InAIP, InNP, InNAs, InNSb, InPAs, InPSb, and mixtures thereof, and a quaternary compound selected from the group consisting of GaAINP, GaAINAs, GaAINSb, GaAIPAs, GaAIPSb, GaInNP, GaInNAs, GaInNSb, GaInPAs, GaInPSb, InAINP, InAINAs, InAINSb, InAIPAs, InAIPSb, and mixtures thereof. In some embodiments, the Group III-V compound may further include a Group II metal. For example, InZnP, etc. may be selected as a Group III-II-V compound.

The Group II-IV-V compound may be a ternary compound selected from the group consisting of ZnSnP, ZnSnP₂, ZnSnAs₂, ZnGeP₂, ZnGeAs₂, CdSnP₂, CdGeP₂ and mixtures thereof.

The Group IV-VI compound may be selected from the group consisting of a binary compound selected from the group consisting of SnS, SnSe, SnTe, PbS, PbSe, PbTe, and mixtures thereof; a ternary compound selected from the group consisting of SnSeS, SnSeTe, SnSTe, PbSeS, PbSeTe, PbSTe, SnPbS, SnPbSe, SnPbTe, and mixtures thereof, and a quaternary compound selected from the group consisting of SnPbSSe, SnPbSeTe, SnPbSTe, and mixtures thereof. The Group IV element may be selected from the group consisting of Si, Ge, and a mixture thereof. The Group IV compound may be a binary compound selected from the group consisting of SiC, SiGe, and a mixture thereof.

In this case, the binary compound, the ternary compound, and/or the quaternary compound may be present in particles having a substantially uniform concentration distribution, or may be present in the same particles having a partially different concentration distribution. In some embodiments, a core/shell structure in which one quantum dot surrounds another quantum dot may be present. The core/shell structure may have a concentration gradient in which the concentration of an element present in the shell becomes lower towards the core.

In some embodiments, a quantum dot may have the core/shell structure including a core having nano-crystals, and a shell surrounding the core, which are described above. The shell of the quantum dot may serve as a protection layer to prevent or reduce the chemical deformation of the core so as to keep semiconductor properties, and/or a charging layer to impart electrophoresis properties to the quantum dot. The shell may be a single layer or multiple layers. Examples of the shell of the quantum dot may include a metal oxide, a non-metal oxide, a semiconductor compound, and combinations thereof.

For example, the metal oxide and the non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, CO₃O₄, and/or NiO, or a ternary compound such as MgAl₂O₄, CoFe₂O₄, NiFe₂O₄, and/or CoMn₂O₄, but the embodiment of the present disclosure is not limited thereto.

In some embodiments, the semiconductor compound may be, for example, CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AIAs, AIP, AISb, etc., but the embodiment of the present disclosure is not limited thereto.

The quantum dot may have, in an emission wavelength spectrum, a full width of half maximum (FWHM) of about 45 nm or less, for example, about 40 nm or less, or about 30 nm or less, and in any of these ranges, the color purity and/or the color reproducibility may be improved. In some embodiments, light emitted through the quantum dot is emitted in all directions, and thus a wide viewing angle may be improved.

In some embodiments, the form of a quantum dot is not particularly limited as long as it is a suitable form in the art, but for example, a quantum dot in the form of spherical, pyramidal, multi-arm, and/or cubic nanoparticles, nanotubes, nanowires, nanofibers, nanoplatelets, and/or the like may be used.

The quantum dot may control the colors of emitted light according to the particle size thereof, and thus the quantum dot may have various light emitting colors such as blue, red, green, and/or the like.

The emission layer EML may have, for example, a thickness of about 100 Å to about 1000 Å, or about 100 Å to about 300 Å. The emission layer EML may have a single layer formed of a single material, a single layer formed of a plurality of different materials, or a multilayer structure having a plurality of layers formed of a plurality of different materials.

Referring back to FIGS. 3 to 7, the first electrode EL1 may have conductivity. The first electrode EL1 may be formed of a metal material, a metal alloy, and/or a conductive compound. The first electrode EL1 may be an anode or a cathode. However, the embodiment of the present disclosure is not limited thereto. In some embodiments, the first electrode EL1 may be a pixel electrode. The first electrode EL1 may be a transmissive electrode, a transflective electrode, or a reflective electrode. The first electrode may include at least one selected from Ag, Mg, Cu, Al, Pt, Pd, Au, Ni, Nd, Ir, Cr, Li, Ca, LiF, Mo, Ti, W, In, Sn, and Zn, compounds of two or more selected therefrom, mixtures of two or more selected therefrom, and oxides thereof.

When the first electrode EL1 is the transmissive electrode, the first electrode EL1 may include a transparent metal oxide such as indium tin oxide (ITO), indium zinc oxide (IZO), zinc oxide (ZnO), and/or indium tin zinc oxide (ITZO). When the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stack structure of LiF and Ca), LiF/Al (a stack structure of LiF and Al), Mo, Ti, W, a compound thereof, or a mixture thereof (e.g., a mixture of Ag and Mg). In some embodiments, the first electrode EL1 may have a multilayer structure including a reflective film or a transflective film formed of any of the above-described materials, and a transparent conductive film formed of indium tin oxide (ITO), indium zinc oxide (IZO), zinc oxide (ZnO), indium tin zinc oxide (ITZO), and/or the like. For example, the first electrode EL1 may have a three-layer structure of ITO/Ag/ITO, but is not limited thereto. In some embodiments, the first electrode EL1 may include any of the above-described metal materials, a combination of two or more metal materials selected from the above-described metal materials, and/or an oxide of the above-described metal materials, but the embodiment of the present disclosure is not limited thereto. The first electrode EL1 may have a thickness of about 700 Å to about 10000 Å. For example, the first electrode EL1 may have a thickness of 1000 Å to about 3000 Å.

The hole transport region HTR may be provided on the first electrode EL1. The hole transport region HTR may include at least one selected from among a hole injection layer HIL, a hole transport layer HTL, a buffer layer, a light emitting auxiliary layer, and an electron blocking layer EBL. In some embodiments, the hole transport region HTR may include a first hole transport layer HTL-1 and a second hole transport layer HTL-2. Hereinafter, descriptions of the hole transport layer HTL may also be applied to the first hole transport layer HTL-1 and the second hole transport layer HTL-2.

For example, at least one of the hole injection layer HIL, the hole transport layer HTL, or the electron blocking layer EBL may include the heterocyclic compound of one or more embodiments. When the heterocyclic compound of one or more embodiments is provided to the electron blocking layer EBL, the electron blocking layer EBL may also serve as an auxiliary emission layer. For example, when the electron blocking layer EBL serves as the auxiliary emission layer, the electron blocking layer EBL may compensate for resonance distance of target light and regulate charge balance to increase light efficiency.

The hole transport region HTR may have a single layer formed of a single material, a single layer formed of a plurality of different materials, or a multilayer structure having a plurality of layers formed of a plurality of different materials. For example, the hole transport region HTR may have a single-layer structure formed of the hole injection layer HIL or the hole transport layer HTL, or a single-layer structure formed of a hole injection material and/or a hole transport material.

For example, the hole transport region HTR may have a single-layer structure formed of a plurality of different materials, or a structure in which a hole injection layer HIL/hole transport layer HTL, a hole injection layer HIL/hole transport layer HTL/buffer layer, a hole injection layer HIL/buffer layer, a hole transport layer HTL/buffer layer, or a hole injection layer HIL/hole transport layer HTL/electron blocking layer EBL are stacked in order from the first electrode EL1. However, this is presented as an example, and the embodiment of the present disclosure is not limited thereto.

The hole transport region HTR may be formed using one or more suitable methods such as a vacuum deposition method, a spin coating method, a cast method, a Langmuir-Blodgett (LB) method, an inkjet printing method, a laser printing method, and/or a laser induced thermal imaging (LITI) method.

The hole transport region HTR may include a compound represented by Formula H-1 below.

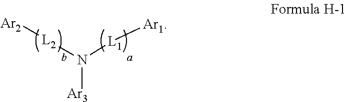

In Formula H-1 above, L₁ and L2 may each independently be a direct linkage, a substituted or unsubstituted arylene group having 6 to 30 ring-forming carbon atoms, or a substituted or unsubstituted heteroarylene group having 2 to 30 ring-forming carbon atoms. a and b may each independently be an integer of 0 to 10. In some embodiments, when a or b is an integer of 2 or greater, a plurality of Li's and L2's may each independently be a substituted or unsubstituted arylene group having 6 to 30 ring-forming carbon atoms, or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. In some embodiments, in Formula H-1, Ar₃ may be a substituted or unsubstituted aryl group having 6 to 30 ring-forming carbon atoms.

A compound represented by Formula H-1 above may be a monoamine compound. In some embodiments, the compound represented by Formula H-1 may be a diamine compound in which at least one selected from among Ar₁ to Ar₃ includes an amine group as a substituent. In some embodiments, the compound represented by Formula H-1 may be a carbazole-based compound including a substituted or unsubstituted carbazole group in at least one of Ar₁ or Ar₂, or a substituted or unsubstituted fluorene-based group in at least one of Ar₁ or Ar_{2.}

The compound represented by Formula H-1 may be represented by any one selected from among compounds from Compound Group H below. However, the compounds listed in Compound Group H below are presented as an example, and the compound represented by Formula H-1 is not limited to the those listed in Compound Group H below.

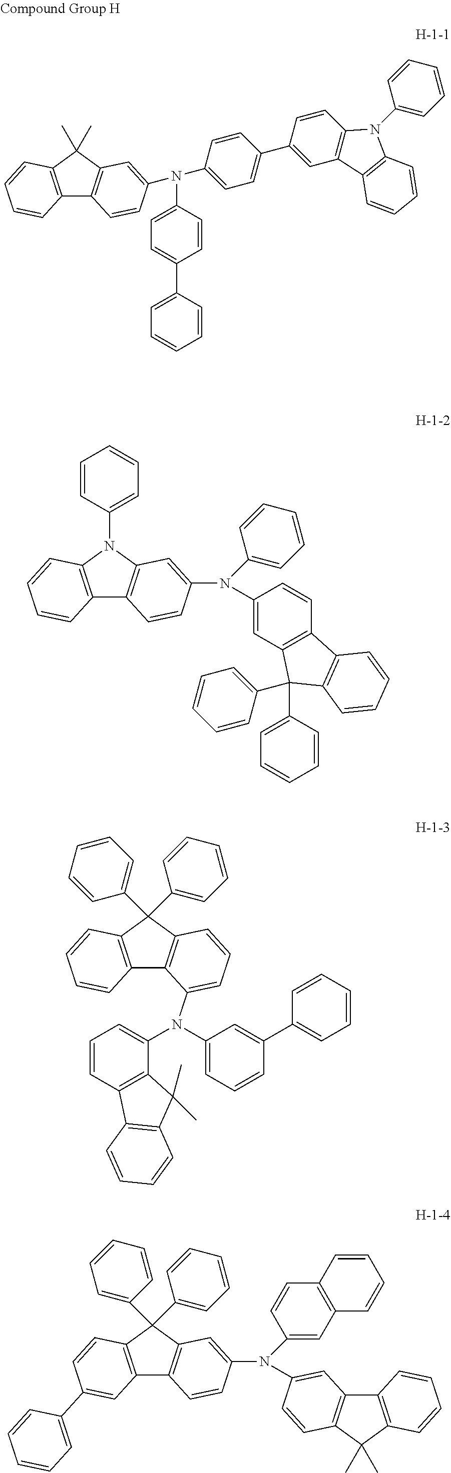 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

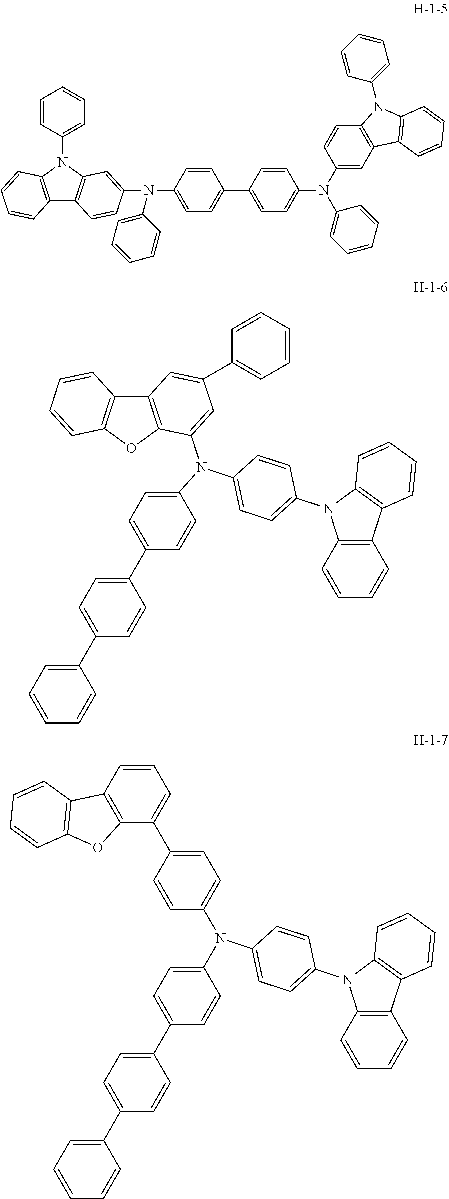 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

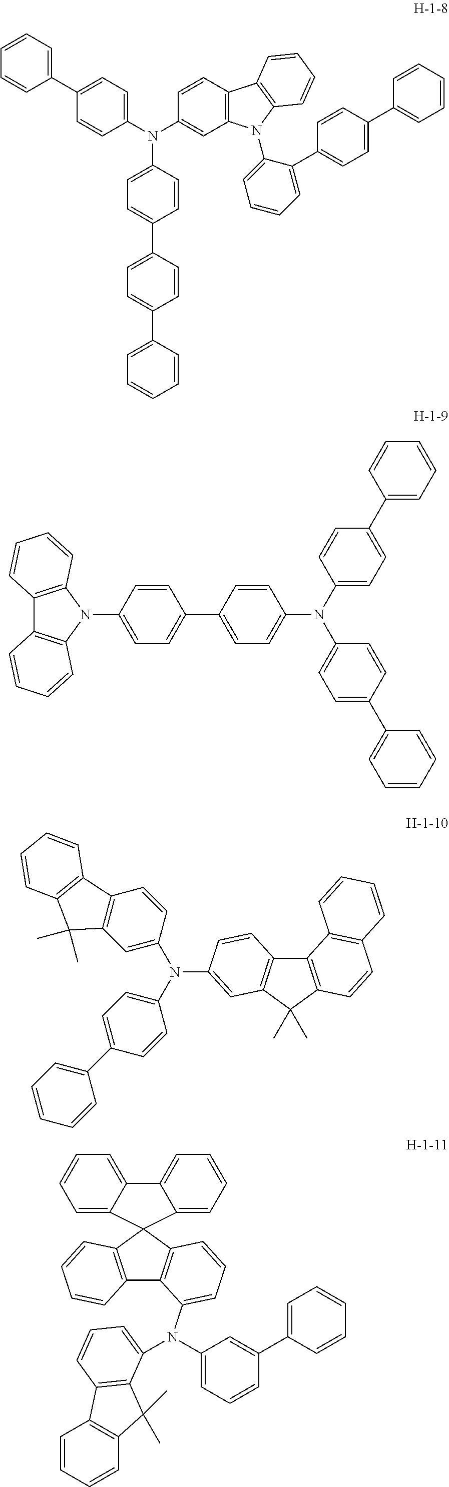

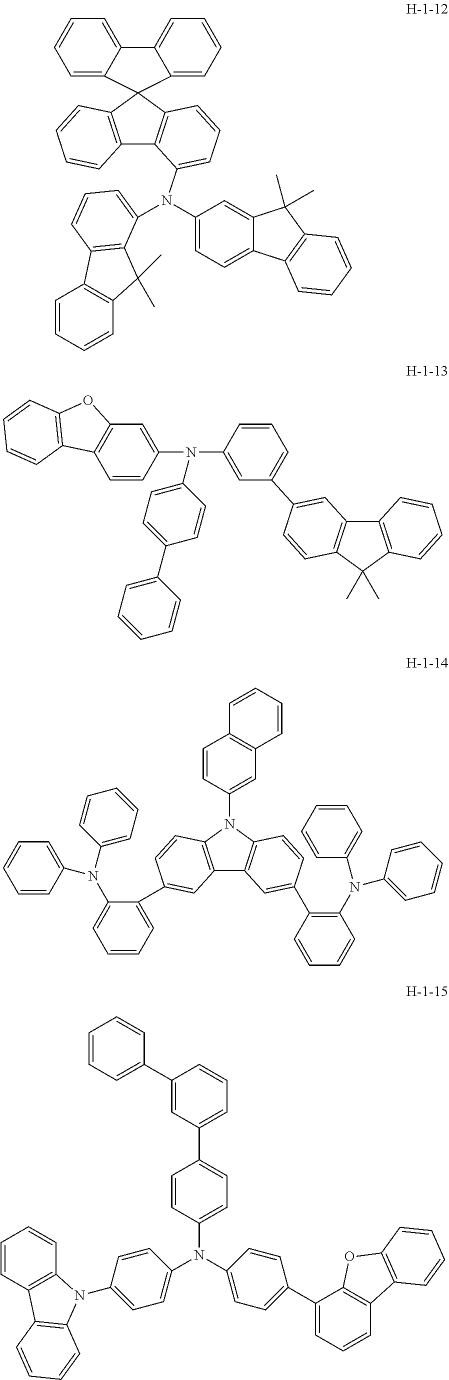 [SMILES: CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21 ; c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1]

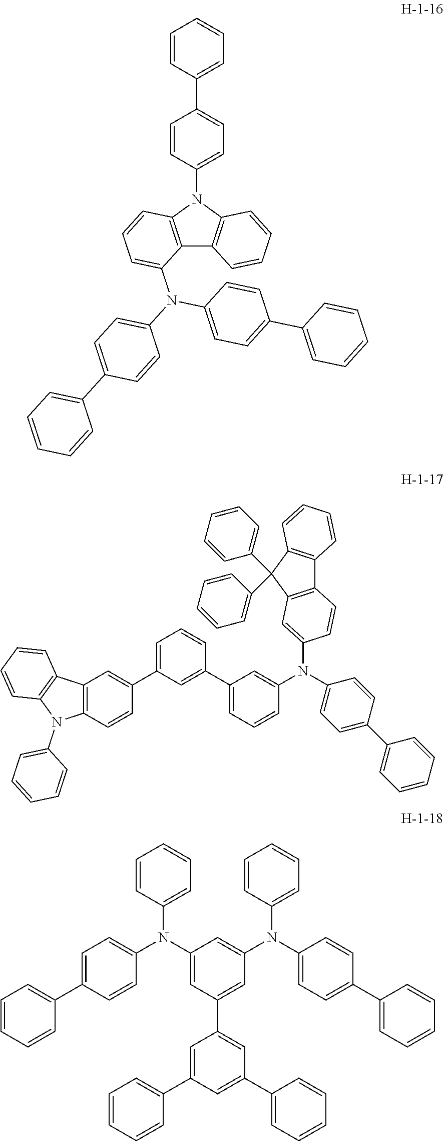 [SMILES: c1ccc(-c2ccc(N(c3ccc(-c4ccccc4)cc3)c3cccc4c3c3ccccc3n4-c3ccc(-c4ccccc4)cc3)cc2)cc1 ; c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1]

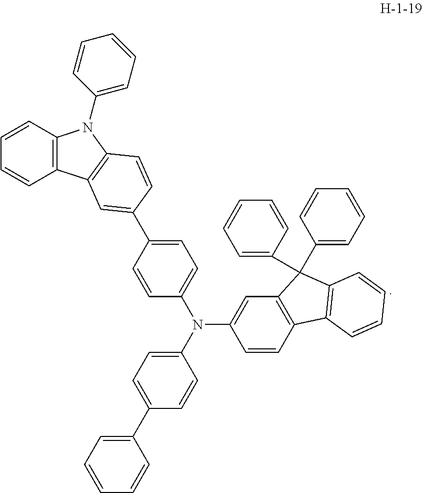 [SMILES: c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may include a phthalocyanine compound (such as copper phthalocyanine), N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine (m-MTDATA), 4,4′4″-tris(N,N-diphenylamino)triphenylamine (TDATA), 4,4′,4″-tris[N-(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonicacid (PANI/CSA), polyaniline/poly(4-styrenesulfonate) (PANI/PSS), N,N′-di(naphthalene-1-yl)-N,N′-diphenyl-benzidine (NPB), triphenylamine-containing polyetherketone (TPAPEK), 4-isopropyl-4′-methyldiphenyliodonium tetrakis(pentafluorophenyl)borate, dipyrazino[2,3-f: 2′,3′-h] quinoxaline-2,3,6,7,10,11-hexacarbonitrile (HATCN), and/or the like.

In some embodiments, the hole transport region HTR may include carbazole-based derivatives (such as N-phenyl carbazole and/or polyvinyl carbazole), fluorene-based derivatives, N,N′-bis(3-methylphenyl)-N,N′-diphenyl-[1,1′-biphenyl]-4,4′-diamine (TPD), triphenylamine-based derivatives (such as 4,4′,4″-tris(N-carbazolyl)triphenylamine (TCTA)), 4,4′-cyclohexylidene bis[N,N-bis(4-methylphenyl]benzenamine] (TAPC), 4,4′-bis[N,N′-(3-tolyl)amino]-3,3′-dimethylbiphenyl (HMTPD), 9-(4-tert-butylphenyl)-3,6-bis(triphenylsilyl)-9H-carbazole (CzSi), 9-phenyl-9H-3,9′-bicarbazole (CCP), 1,3-bis(N-carbazol-9-yl)benzene (mCP), 1,3-bis(1,8-dimethyl-9H-carbazol-9-yl)benzene (mDCP), 9-[3-(triphenylsilyl)phenyl]-9H-3,9′-bicarbazole (SiCzCz), and/or the like.

The hole transport region HTR may include the compounds of the hole transport region described above in at least one selected from among the hole injection layer HIL, the hole transport layer HTL, and the electron blocking layer EBL.

The hole transport region HTR may further include, in addition to the above-described materials, a charge generation material to increase conductivity. The charge generation material may be substantially uniformly or non-substantially uniformly dispersed in the hole transport region HTR. The charge generation material may be, for example, a p-dopant. The p-dopant may include at least one of halogenated metal compounds, quinone derivatives, metal oxides, or cyano group-containing compounds, but is not limited thereto. For example, the p-dopant may include halogenated metal compounds (such as Cul and/or Rbl), quinone derivatives (such as tetracyanoquinodimethane (TCNQ) and/or 2,3,5,6-tetrafluoro-7,7′,8,8′-tetracyanoquinodimethane (F4-TCNQ)), metal oxides (such as tungsten oxide and/or molybdenum oxide), cyano group-containing compounds (such as dipyrazino[2,3-f: 2′,3′-h] quinoxaline-2,3,6,7,10,11-hexacarbonitrile (HATCN) and/or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9)), and/or the like, but is not limited thereto.

As described above, the hole transport region HTR may further include at least one of a buffer layer or an electron blocking layer EBL in addition to the hole injection layer HIL and the hole transport layer HTL. The buffer layer may compensate a resonance distance according to wavelengths of light emitted from an emission layer EML, and may thus increase light emitting efficiency. Material(s) which may be included in the hole transport region HTR may be used as material(s) included in the buffer layer. The electron blocking layer EBL is a layer that serves to prevent or reduce the injection of electrons from the electron transport region ETR to the hole transport region HTR.

The hole transport region HTR may have, for example, a thickness of about 50 Å to about 15000 Å. The hole transport region HTR may have a thickness of about 100 Å to about 10000 Å, for example, about 100 Å to about 5000 Å. When the hole transport region HTR includes the hole injection layer HIL, the hole injection layer HIL may have a thickness of, for example, about 30 Å to about 1000 Å. When the hole transport region HTR includes the hole transport layer HTL, the hole transport layer HTL may have a thickness of about 30 Å to about 1000 Å. When the hole transport region HTR includes the electron blocking layer EBL, the electron blocking layer EBL may have a thickness of, for example, about 10 Å to about 1000 Å. When the thicknesses of the hole transport region HTR, the hole injection layer HIL, the hole transport layer HTL, and/or the electron blocking layer EBL satisfy their respective above-described ranges, satisfactory or suitable hole transport properties may be obtained without a substantial increase in driving voltage.

In the light emitting element ED of one or more embodiments, the electron transport region ETR may be provided on the emission layer EML. The electron transport region ETR may include at least one selected from among a hole blocking layer HBL, an electron transport layer ETL, and an electron injection layer EIL, but the embodiment of the present disclosure is not limited thereto.

The electron transport region ETR may have a single layer formed of a single material, a single layer formed of a plurality of different materials, or a multilayer structure having a plurality of layers formed of a plurality of different materials. For example, the electron transport region ETR may have a single layer structure of an electron injection layer EIL or an electron transport layer ETL, and may have a single layer structure formed of an electron injection material and/or an electron transport material. In some embodiments, the electron transport region ETR may have a single layer structure formed of a plurality of different materials, or may have a structure in which an electron transport layer ETL/electron injection layer EIL, or a hole blocking layer HBL/electron transport layer ETL/electron injection layer EIL are stacked in order from the emission layer EML, but is not limited thereto. The electron transport region ETR may have a thickness of, for example, about 1000 Å to about 1500 Å.

The electron transport region ETR may be formed using one or more suitable methods such as a vacuum deposition method, a spin coating method, a cast method, a Langmuir-Blodgett (LB) method, an inkjet printing method, a laser printing method, and/or a laser induced thermal imaging (LITI) method.

The electron transport region ETR may include a compound represented by Formula ET-1 described above. The electron transport region ETR may include an anthracene-based compound. However, the embodiment of the present disclosure is not limited thereto, and the electron transport region ETR may include, for example, tris(8-hydroxyquinolinato)aluminum (Alq3), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-ylphenyl)-9,10-dinaphthylanthracene, 1,3,5-tri(1-phenyl-1H-benzo[d]imidazol-2-yl)benzene (TPBi), 2,9-dimethyl-4,7-diphenyl-1, 10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,08)-(1,1′-biphenyl-4-olato)aluminum (BAIq), berylliumbis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), or a mixture thereof.

In some embodiments, the electron transport region ETR may include halogenated metals (such as LiF, NaCl, CsF, RbC, Rbl, Cul, and/or KI), lanthanide metals such as Yb, or co-deposition material(s) of a halogenated metal and a lanthanide metal. For example, the electron transport region ETR may include KI:Yb, Rbl:Yb, LiF:Yb, and/or the like as a co-deposition material. In some embodiments, for the electron transport region ETR, a metal oxide such as Li₂O and/or BaO, 8-hydroxyl-lithium quinolate (Liq), and/or the like may be used, but the embodiment of the present disclosure is limited thereto. The electron transport region ETR may also be formed of a mixture material of an electron transport material and an insulating organo-metal salt. The organo metal salt may be a material having an energy band gap of about 4 eV or greater. For example, the organo-metal salt may include, for example, one or more selected from among metal acetates, metal benzoates, metal acetoacetates, metal acetylacetonates, and metal stearates.

The electron transport region ETR may further include, for example, at least one selected from among 2,9-dimethyl-4,7-diphenyl-1, 10-phenanthroline (BCP), diphenyl(4-(triphenylsilyl)phenyl)phosphine oxide (TSPO1), and 4,7-diphenyl-1,10-phenanthroline (Bphen), in addition to the materials described above, but the embodiment of the present disclosure is not limited thereto. The electron transport region ETR may include the compounds of the electron transport region described above in at least one selected from among the electron injection layer EIL, the electron transport layer ETL, and the hole blocking layer HBL.

When the electron transport region ETR includes the electron transport layer ETL, the electron transport layer ETL may have a thickness of about 100 Å to about 1000 Å, for example, about 150 Å to about 500 Å. When the thickness of the electron transport layer ETL satisfies any of the above-described ranges, satisfactory or suitable electron transport properties may be obtained without a substantial increase in driving voltage. When the electron transport region ETR includes the electron injection layer EIL, the electron injection layer EIL may have a thickness of about 1 Å to about 100 Å, for example, about 3 Å to about 90 Å. When the thickness of the electron injection layer EIL satisfies any of the above-described ranges, satisfactory or suitable electron injection properties may be obtained without a substantial increase in driving voltage.

The second electrode EL2 may be provided on the electron transport region ETR. The second electrode EL2 may be a common electrode. The second electrode EL2 may be a cathode or an anode but the embodiment of the present disclosure is not limited thereto. For example, when the first electrode EL1 is an anode, the second electrode EL2 may be a cathode, and when the first electrode EL1 is a cathode, the second electrode EL2 may be an anode. The second electrode EL2 may include at least one selected from Ag, Mg, Cu, Al, Pt, Pd, Au, Ni, Nd, Ir, Cr, Li, Ca, LiF, Mo, Ti, W, In, Sn, and Zn, compounds of two or more selected therefrom, mixtures of two or more selected therefrom, and oxides thereof.

The second electrode EL2 may be a transmissive electrode, a transflective electrode, or a reflective electrode. When the second electrode EL2 is a transmissive electrode, the second electrode EL2 may be formed of a transparent metal oxide, for example, indium tin oxide (ITO), indium zinc oxide (IZO), zinc oxide (ZnO), indium tin zinc oxide (ITZO), and/or the like.

When the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, a compound thereof, and/or a mixture thereof (e.g., AgMg, AgYb, and/or MgYb). In some embodiments, the second electrode EL2 may have a multilayer structure including a reflective film or a transflective film formed of any of the above-described materials, and a transparent conductive film formed of indium tin oxide (ITO), indium zinc oxide (IZO), zinc oxide (ZnO), indium tin zinc oxide (ITZO), and/or the like. For example, the second electrode EL2 may include any of the above-described metal materials, a combination of two or more metal materials selected from the above-described metal materials, and/or an oxide of any of the above-described metal materials.

In some embodiments, the second electrode EL2 may be connected with an auxiliary electrode. When the second electrode EL2 is connected with the auxiliary electrode, the resistance of the second electrode EL2 may decrease.

In some embodiments, a capping layer CPL may be further provided on the second electrode EL2 of the light emitting element ED according to one or more embodiments. The capping layer CPL may include a multilayer or a single layer.

In one or more embodiments, the capping layer CPL may be an organic layer or an inorganic layer. For example, when the capping layer CPL includes an inorganic material, the inorganic material may include an alkali metal compound such as LiF, an alkaline earth metal compound such as MgF₂, SiON, SiNx, SiOy, and/or the like.

For example, when the capping layer CPL includes an organic material, the organic material may include a-NPD, NPB, TPD, m-MTDATA, Alq3 CuPc, N4,N4,N4′,N4′-tetra(biphenyl-4-yl) biphenyl-4,4′-diamine (TPD15), 4,4′,4″-tris(carbazol-9-yl)triphenylamine (TCTA), and/or the like, and/or may include epoxy resins and/or acrylates such as methacrylates. However, the embodiment of the present disclosure is not limited thereto, and the capping layer CPL may include compounds P1 to P5 below.

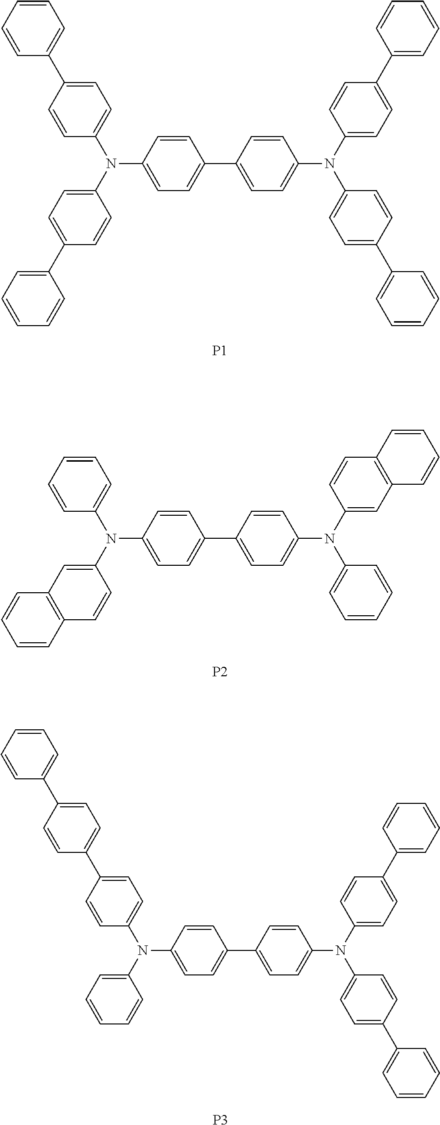 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; "P1" ; c1ccc(N(c2ccc(-c3ccc(N(c4ccccc4)c4ccc5ccccc5c4)cc3)cc2)c2ccc3ccccc3c2)cc1 ; "P2" ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; "P3"]

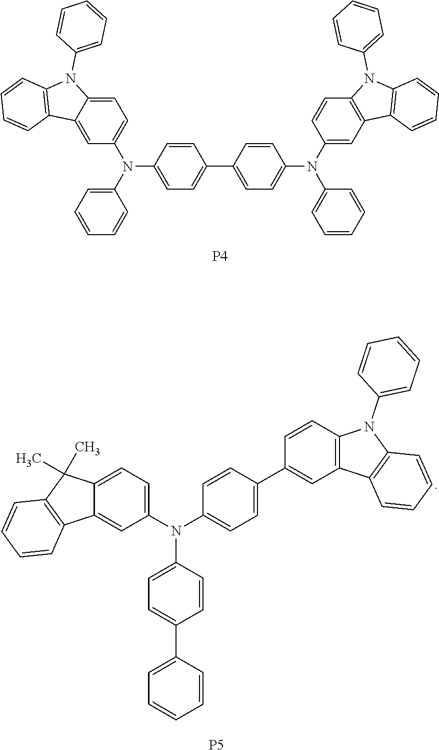

In some embodiments, the capping layer CPL may have a refractive index of about 1.6 or greater. For example, the capping layer CPL may have a refractive index of about 1.6 or greater in a wavelength range of about 550 nm to about 660 nm.

FIGS. 8 to 11 are each a cross-sectional view of a display device according to one or more embodiments. Hereinafter, in the description of the display device according to one or more embodiments with reference to FIGS. 8 to 11, content overlapping the one described above with reference to FIGS. 1 to 7 will not be described again, and the differences will be mainly described.

Referring to FIG. 8, a display device DD_a according to one or more embodiments may include a display panel DP having a display element layer DP-ED, a light control layer CCL provided on the display panel DP, and a color filter layer CFL. In one or more embodiments shown in FIG. 8, the display panel DP may include a base layer BS, a circuit layer DP-CL provided on the base layer BS, and a display element layer DP-ED, and the element layer DP-ED may include a light emitting element ED.

The light emitting element ED may include a first electrode EL1, a hole transport region HTR provided on the first electrode EL1, an emission layer EML provided on the hole transport region HTR, an electron transport region ETR provided on the emission layer EML, and a second electrode EL2 provided on the electron transport region ETR. In some embodiments, a structure of the light emitting element ED shown in FIG. 8 may be the same as the structure of the light emitting element of FIGS. 3 to 7 described above. The light emitting element ED shown in FIG. 8 may include the heterocyclic compound of one or more embodiments.

Referring to FIG. 8, the emission layer EML may be provided in the openings OH defined in the pixel defining films PDL. For example, the emission layer EML separated by the pixel defining films PDL and provided corresponding to each of light emitting regions PXA-R, PXA-G, and PXA-B may emit light in the same wavelength ranges. In the display device DD-a of one or more embodiments, the emission layer EML may emit blue light. In some embodiments, the emission layer EML may be provided as a common layer throughout the light emitting regions PXA-R, PXA-G, and PXA-B.

The light control layer CCL may be provided on the display panel DP. The light control layer CCL may include a light converter. The light converter may be a quantum dot and/or a phosphor. The light converter may wavelength-convert the provided light and emit the wavelength-converted light. For example, the light control layer CCL may be a layer containing quantum dots and/or phosphors.

The light control layer CCL may include a plurality of light control units CCP1, CCP2, and CCP3. The light control units CCP1, CCP2, and CCP3 may be spaced apart from each other.

Referring to FIG. 8, a division pattern BMP may be provided between the light control units CCP1, CCP2, and CCP3 spaced apart from each other, but the embodiment of the present disclosure is not limited thereto. In FIG. 7, the division pattern BMP is shown to non-overlap the light control units CCP1, CCP2, and CCP3, but edges of the light control units CCP1, CCP2, and CCP3 may overlap at least a portion of the division pattern BMP.

The light control layer CCL may include a first light control unit CCP1 including a first quantum dot QD1 for converting first color light provided from the light emitting element ED into second color light, a second light control unit CCP2 including a second quantum dot QD2 for converting the first color light into third color light, and a third light control unit CCP3 for transmitting the first color light. In one or more embodiments, the first light control unit CCP1 may provide red light, which is the second color light, and the second light control unit CCP2 may provide green light, which is the third color light. The third light control unit CCP3 may transmit and provide blue light, which is the first color light provided from the light emitting element ED. For example, the first quantum dot QD1 may be a red quantum dot and the second quantum dot QD2 may be a green quantum dot. The same descriptions as above may be applied to the quantum dots QD1 and QD2.

In some embodiments, the light control layer CCL may further include scatterers SP. The first light control unit CCP1 may include the first quantum dot QD1 and the scatterers SP, the second light control unit CCP2 may include the second quantum dot QD2 and the scatterers SP, and the third light control unit CCP3 may not include a quantum dot but may include the scatterers SP.

The scatterers SP may be inorganic particles. For example, the scatterers SP may include at least one selected from among TiO₂, ZnO, Al₂O₃, SiO₂, and hollow silica. The scatterers SP may include any one selected from among TiO₂, ZnO, Al₂O₃, SiO₂, and hollow silica, or may be a mixture of two or more materials selected from among TiO₂, ZnO, Al₂O₃, SiO₂, and hollow silica.

The first light control unit CCP1, the second light control unit CCP2, and the third light control unit CCP3 may each include base resins BR1, BR2, and BR3 for dispersing the quantum dots QD1 and QD2 and the scatterers SP. In one or more embodiments, the first light control unit CCP1 may include the first quantum dot QD1 and the scatterers SP dispersed in the first base resin BR1, the second light control unit CCP2 may include the second quantum dot QD2 and the scatterers SP dispersed in the second base resin BR2, and the third light control unit CCP3 may include the scatterers SP dispersed in the third base resin BR3.

The base resins BR1, BR2, and BR3 are a medium in which the quantum dots QD1 and QD2 and the scatterers SP are dispersed, and may be formed of one or more suitable resin compositions, which may be generally referred to as a binder. For example, the base resins BR1, BR2, and BR3 may be an acrylic-based resin, a urethane-based resin, a silicone-based resin, an epoxy-based resin, etc. The base resins BR1, BR2, and BR3 may be a transparent resin. In one or more embodiments, the first base resin BR1, the second base resin BR2, and the third base resin BR3 may each be the same as or different from each other.

The light control layer CCL may include a barrier layer BFL1. The barrier layer BFL1 may serve to prevent or reduce the introduction of moisture and/or oxygen (hereinafter referred to as “moisture/oxygen”). The barrier layer BFL1 may prevent or reduce the exposure of the light control units CCP1, CCP2, and CCP3 to moisture/oxygen. In some embodiments, the barrier layer BFL1 may cover the light control units CCP1, CCP2, and CCP3. In some embodiments, a barrier layer BFL2 may be provided between the light control units CCP1, CCP2, and CCP3 and the color filter layer CFL.

The barrier layers BFL1 and BFL2 may include at least one inorganic layer. For example, the barrier layers BFL1 and BFL2 may be formed of an inorganic material. For example, the barrier layers BFL1 and BFL2 may be formed including silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, a metal thin film in which light transmittance is secured, and/or the like. In some embodiments, the barrier layers BFL1 and BFL2 may further include an organic film. The barrier layers BFL1 and BFL2 may be formed of a single layer or a plurality of layers.

In the display device DD-a of one or more embodiments, the color filter layer CFL may be provided on the light control layer CCL. For example, the color filter layer CFL may be directly provided on the light control layer CCL. In this case, the barrier layer BFL2 may not be provided (e.g., may be omitted).

The color filter layer CFL may include first to third filters CF1, CF2, and CF3. The first to third filters CF1, CF2, and CF3 may be provided corresponding to the red light emitting region PXA-R, the green light emitting region PXA-G, and the blue light emitting region PXA-B, respectively.

For example, the color filter layer CFL may include a first filter CF1 for transmitting second color light, a second filter CF2 for transmitting third color light, and a third filter CF3 for transmitting first color light. For example, the first filter CF1 may be a red filter, the second filter CF2 may be a green filter, and the third filter CF3 may be a blue filter. The filters CF1, CF2, and CF3 may each include a polymer photosensitive resin, a pigment and/or a dye. The first filter CF1 may include a red pigment and/or a red dye, the second filter CF2 may include a green pigment and/or a green dye, and the third filter CF3 may include a blue pigment and/or a blue dye.

In some embodiments, the embodiment of the present disclosure is not limited thereto, and the third filter CF3 may not include a pigment or a dye. The third filter CF3 may include a polymer photosensitive resin, but not include a pigment or a dye. The third filter CF3 may be transparent. The third filter CF3 may be formed of a transparent photosensitive resin.

In some embodiments, in one or more embodiments, the first filter CF1 and the second filter CF2 may be yellow filters. The first filter CF1 and the second filter CF2 may not be separated and may be provided as a single body.

In one or more embodiments, the color filter layer CFL may further include a light blocking unit. The light blocking unit may be a black matrix. The light blocking unit may be formed including an organic light blocking material and/or an inorganic light blocking material, both including a black pigment and/or a black dye. The light blocking unit may prevent or reduce light leakage, and separate boundaries between the adjacent filters CF1, CF2, and CF3.

The base substrate BL may be provided on the color filter layer CFL. The base substrate BL may be a member providing a base surface on which the color filter layer CFL and the light control layer CCL are provided. The base substrate BL may be a glass substrate, a metal substrate, a plastic substrate, and/or the like. However, the embodiment of the present disclosure is not limited thereto, and the base substrate BL may be an inorganic layer, an organic layer, or a composite material layer (e.g., including an organic material and an inorganic material). In some embodiments, the base substrate BL may not be provided (e.g., may be omitted).

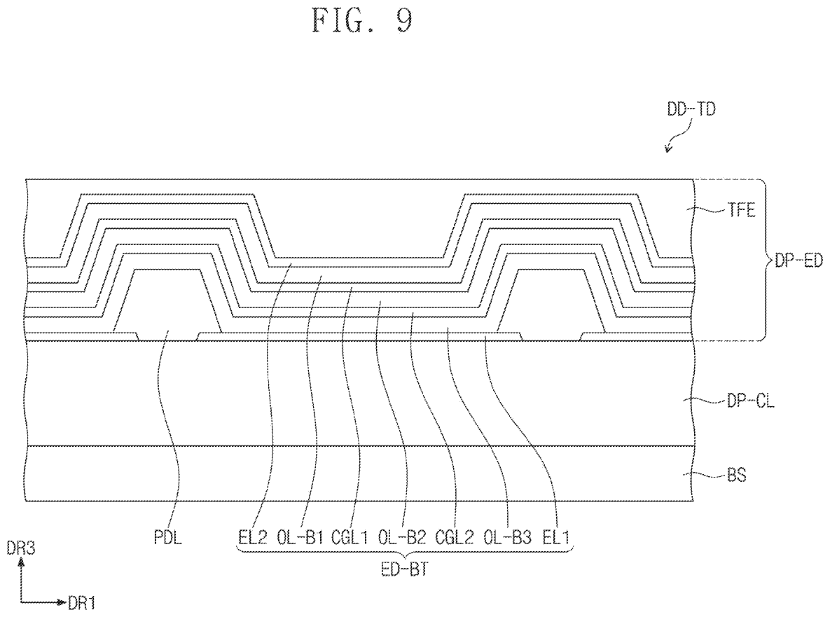

FIG. 9 is a cross-sectional view showing a portion of a display device according to one or more embodiments. FIG. 9 shows another embodiment of a portion corresponding to the display panel DP of FIG. 8.

In a display device DD-TD of one or more embodiments, a light emitting element ED-BT may include a plurality of light emitting structures OL-B1, OL-B2, and OL-B3. The light emitting element ED-BT may include the first electrode EL1 and the second electrode EL2 facing each other, and the plurality of light emitting structures OL-B1, OL-B2, and OL-B3 provided by being sequentially stacked in a thickness direction between the first electrode EL1 and the second electrode EL2. A light emitting element ED-BT may include the heterocyclic compound of one or more embodiments.

The light emitting structures OL-B1, OL-B2, and OL-B3 each may include the emission layer EML (FIG. 8), a hole transport region HTR and an electron transport region ETR provided with the emission layer EML (FIG. 8) therebetween. For example, the light emitting element ED-BT included in the display device DD-TD of one or more embodiments may be a light emitting element having a tandem structure including a plurality of emission layers.

In one or more embodiments shown in FIG. 9, light emitted from each of the light emitting structures OL-B1, OL-B2, and OL-B3 may all be blue light. However, the embodiment of the present disclosure is not limited thereto, and wavelength ranges of light emitted from each of the light emitting structures OL-B1, OL-B2, and OL-B3 may be different from each other. For example, the light emitting element ED-BT including the plurality of light emitting structures OL-B1, OL-B2, and OL-B3 emitting light in different wavelength ranges may emit white light.

Charge generation layers CGL1 and CGL2 may be provided between neighboring light emitting structures OL-B1, OL-B2, and OL-B3. The charge generation layers CGL1 and CGL2 may include a p-type charge generation layer and/or an n-type charge generation layer.

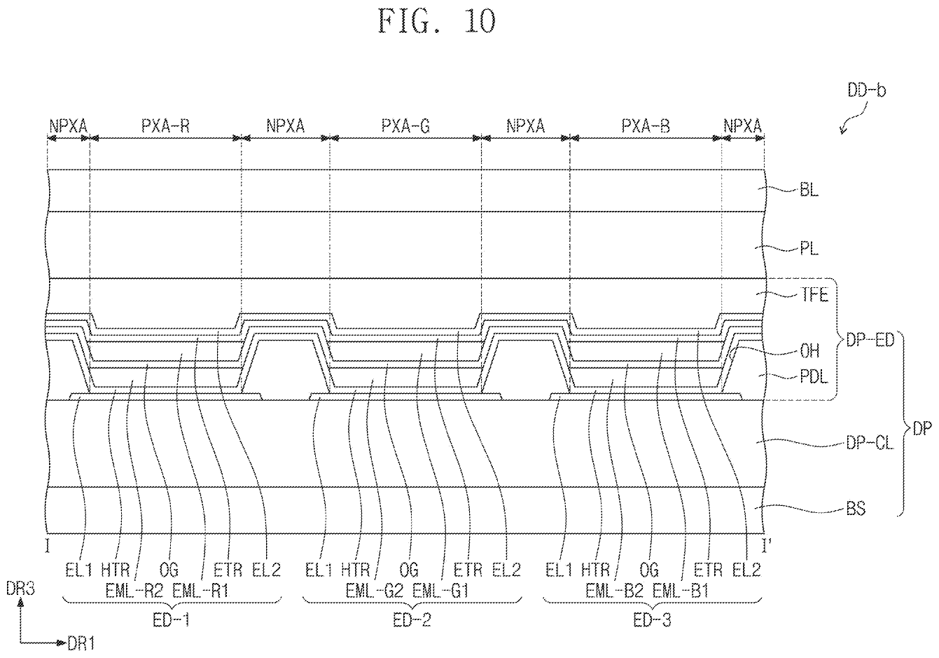

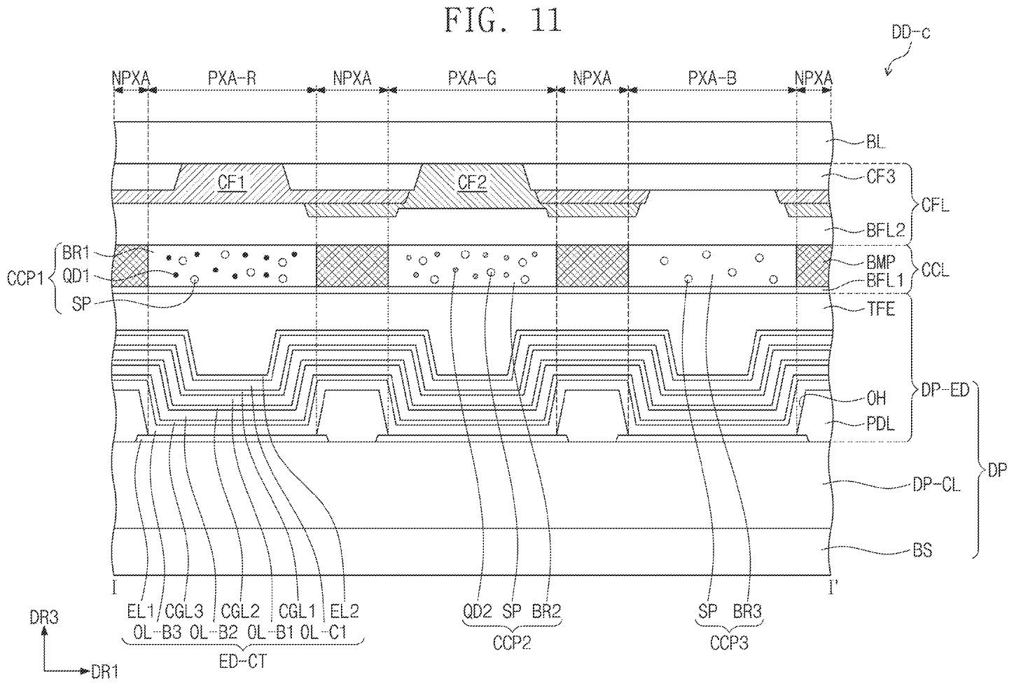

Referring to FIG. 10, a display device DD-b according to one or more embodiments may include light emitting elements ED-1, ED-2, and ED-3 in which two emission layers are stacked. Compared to the display device DD according to one or more embodiments shown in FIG. 2, the difference is that in the one or more embodiments shown in FIG. 10, the first to third light emitting elements ED-1, ED-2, and ED-3 each include two emission layers stacked in a thickness direction. In each of the first to third light emitting elements ED-1, ED-2, and ED-3, the two emission layers may emit light in the same wavelength range. At least one selected from among the first to third light emitting elements ED-1, ED-2, and ED-3 may include the heterocyclic compound of one or more embodiments.

The first light emitting element ED-1 may include a first red emission layer EML-R1 and a second red emission layer EML-R2. The second light emitting element ED-2 may include a first green emission layer EML-G1 and a second green emission layer EML-G2. In some embodiments, the third light emitting element ED-3 may include a first blue emission layer EML-B1 and a second blue emission layer EML-B2.

A light emitting auxiliary portion OG may be provided between the first red emission layer EML-R1 and the second red emission layer EML-R2, between the first green emission layer EML-G1 and the second green emission layer EML-G2, and between the first blue emission layer EML-B1 and the second blue emission layer EML-B2.

The light emitting auxiliary portion OG may include a single layer or multiple layers. The light emitting auxiliary portion OG may include a charge generation layer. For example, the light emitting auxiliary portion OG may include an electron transport region, a charge generation layer, and a hole transport region that are sequentially stacked. The light emitting auxiliary portion OG may be provided as a common layer throughout the first to third light emitting elements ED-1, ED-2, and ED-3. However, the embodiment of the present disclosure is not limited thereto, and the light emitting auxiliary portion OG may be provided to be patterned inside the openings OH defined in the pixel defining films PDL.

The first red emission layer EML-R1, the first green emission layer EML-G1, and the first blue emission layer EML-B1 may be provided between the light emitting auxiliary portion OG and the electron transport region ETR. The second red emission layer EML-R2, the second green emission layer EML-G2, and the second blue emission layer EML-B2 may be provided between the hole transport region HTR and the light emitting auxiliary portion OG.

In one or more embodiments, the light emitting element ED-1 may include the first electrode EL1, the hole transport region HTR, the second red emission layer EML-R2, the emission auxiliary portion OG, the first red emission layer EML-R1, the electron transport region ETR, and the second electrode EL2, which are sequentially stacked. The second light emitting element ED-2 may include the first electrode EL1, the hole transport region HTR, the second green emission layer EML-G2, the emission auxiliary portion OG, the first green emission layer EML-G1, the electron transport region ETR, and the second electrode EL2, which are sequentially stacked. The third light emitting element ED-3 may include the first electrode EL1, the hole transport region HTR, the second blue emission layer EML-B2, the emission auxiliary portion OG, the first blue emission layer EML-B1, the electron transport region ETR, and the second electrode EL2, which are sequentially stacked.

In some embodiments, an optical auxiliary layer PL may be provided on the display element layer DP-ED. The optical auxiliary layer PL may include a polarizing layer. The optical auxiliary layer PL may be provided on the display panel DP to control reflected light in the display panel DP due to external light. In some embodiments, the optical auxiliary layer PL may not be provided (e.g., may be omitted) in the display device according to one or more embodiments.

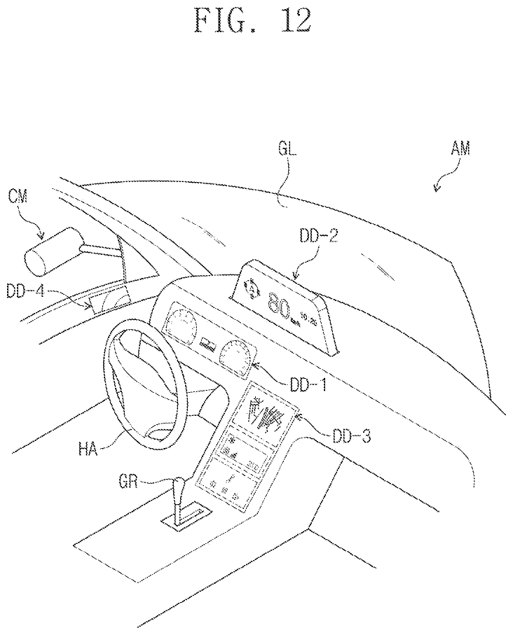

Unlike FIGS. 9 and 10, the display device DD-c of FIG. 11 is illustrated to include four light emitting structures OL-B1, OL-B2, OL-B3, and OL-C1. The light emitting element ED-CT may include the first electrode EL1 and the second electrode EL2 facing each other, and the first to fourth light emitting structures OL-B1, OL-B2, OL-B3, and OL-C1 sequentially stacked in a thickness direction between the first electrode EL1 and the second electrode EL2. At least one selected from among the first to fourth light emitting structures OL-B1, OL-B2, OL-B3, and OL-C1 may include the heterocyclic compound of one or more embodiments.

Among the four light emitting structures, the first to third light emitting structures OL-B1, OL-B2, and OL-B3 may emit blue light, and the fourth light emitting structure OL-C1 may emit green light. However, the embodiment of the present disclosure is not limited thereto, and the first to fourth light emitting structures OL-B1, OL-B2, OL-B3, and OL-C1 may emit light having different wavelength ranges.

Between the third light emitting structure OL-B3, the second light emitting structure OL-B2, the first light emitting structure OL-B1, and the fourth light emitting structure OL-C1, charge generation layers CGL3, CGL2, and CGL1 may be provided. The charge generation layers CGL3, CGL2 and CGL1 provided between the neighboring light emitting structures OL-B3, OL-B2, OL-B1, and OL-C1 may include a p-type charge generation layer (e.g., a p-charge generation layer) and/or an n-type charge generation layer (e.g., an n-charge generation layer).

FIG. 12 is a view showing a vehicle AM in which first to fourth display devices DD-1, DD-2, DD-3, and DD-4 are provided. At least one selected from among the first to fourth display devices DD-1, DD-2, DD-3, and DD-4 may have the same configuration as the display devices DD, DD-TD, DD-a, DD-b, and DD-c of one or more embodiments described with reference to FIGS. 1, 2, and 8 to 11.

FIG. 12 shows a car as the vehicle AM, but this is presented as an example, and the first to fourth display devices DD-1, DD-2, DD-3, and DD-4 may be provided on other means of transportation, such as bicycles, motorcycles, trains, ships, and/or airplanes. In some embodiments, at least one selected from among the first to fourth display devices DD-1, DD-2, DD-3, and DD-4 having the same configuration as any of the display devices DD, DD-TD, DD-a, DD-b, and DD-c of one or more embodiments may be adopted for personal computers, laptop computers, personal digital terminals, game consoles, portable electronic devices, televisions, monitors, outdoor billboards, and/or the like. In some embodiments, these are merely presented as one or more embodiments, and thus the display device may be adopted for other suitable electronic devices without departing from the present disclosure.

At least one selected from among the first to fourth display devices DD-1, DD-2, DD-3, and DD-4 may include the light emitting element ED of one or more embodiments described with reference to FIGS. 3 to 7. The light emitting element ED according to one or more embodiments may include a heterocyclic compound of one or more embodiments. At least one selected from among the first to fourth display devices DD-1, DD-2, DD-3, and DD-4 includes a light emitting element ED containing the heterocyclic compound of one or more embodiments, thereby increasing lifespan.

Referring to FIG. 12, the vehicle AM may include a wheel HA and a gear GR for operating the vehicle AM. In some embodiments, the vehicle AM may include a front window GL provided to face a driver.

The first display device DD-1 may be provided in a first region overlapping the wheel HA. For example, the first display device DD-1 may be a digital cluster displaying first information of the vehicle AM. The first information may include a first scale indicating driving speed of the vehicle AM, a second scale indicating engine revolutions (i.e., revolutions per minute (RPM)), an image indicating fuel gauge, and/or the like. The first scale and the second scale may be displayed as digital images.

The second display device DD-2 may be provided in a second region facing a driver seat and overlapping the front window GL. The driver seat may be a seat in which the wheel HA is provided. For example, the second display device DD-2 may be a head up display HUD displaying second information of the vehicle AM. The second display device DD-2 may be optically transparent. The second information may include digital numbers indicating driving speed of the vehicle AM and may further include information such as current time. In some embodiments, the second information of the second display device DD-2 may be projected and displayed on the front window GL.

The third display device DD-3 may be provided in a third region adjacent to the gear GR. For example, the third display device DD-3 may be a center information display CID for a vehicle, which is provided between a driver seat and a front passenger seat and displays third information. The passenger seat may be a seat spaced apart from the driver seat with the gear GR therebetween. The third information may include information about road conditions (e.g., navigation information), music and/or radio play, dynamic video (and/or image) play, temperature inside the vehicle AM, and/or the like.

The fourth display device DD-4 may be provided in a fourth region spaced apart from the wheel HA and the gear GR and adjacent to a side of the vehicle AM. For example, the fourth display device DD-4 may be a digital side mirror displaying fourth information. The fourth display device DD-4 may display images of conditions outside the vehicle AM, which are taken by a camera module CM provided outside the vehicle AM. The fourth information may include images of conditions outside the vehicle AM.

The first to fourth information described above are presented as an example, and the first to fourth display devices DD-1, DD-2, DD-3, and DD-4 may further display information about inside and/or outside the vehicle AM. The first to fourth information may include different information. However, the embodiments of the present disclosure are not limited thereto, and some of the first to fourth information may include the same information.

Hereinafter, with reference to Examples and Comparative Examples, a heterocyclic compound according to one or more embodiments of the present disclosure and a light emitting element according to one or more embodiments will be described. However, Examples shown below are shown only for the understanding of the present disclosure, and the scope of the present disclosure is not limited thereto.

### EXAMPLES

### 1. Synthesis of Heterocyclic Compounds of Examples

A process of synthesizing heterocyclic compounds according to one or more embodiments of the present disclosure will be described in more detail by presenting a process of synthesizing Compounds, 3, 4, 5, 8, 10, 18, 20, 36, and 39 as examples. However, a process of synthesizing heterocyclic compounds, which will be described hereinafter, is provided as an example, and thus a process of synthesizing compounds according to one or more embodiments of the present disclosure is not limited to Examples below.

### (1) Synthesis of Heterocyclic Compound 3

Heterocyclic compound 3 according to one or more embodiments may be synthesized by, for example, a process of Reaction Formula 1 below.

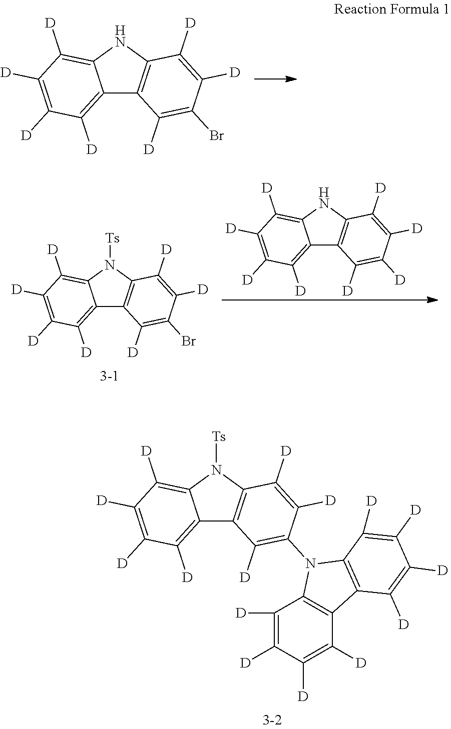

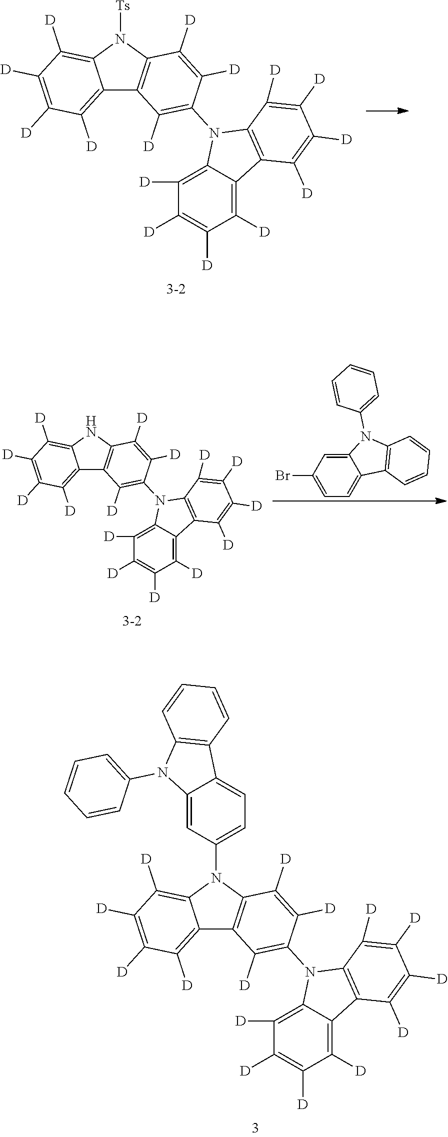

### Synthesis of Intermediate 3-1

3-bromo-9H-carbazole-1,2,4,5,6,7,8-d7 (CAS #=2764814-81-3) (1 eq), TsCl (p-toluenesulfonyl chloride, 1 eq), and KOH (1 eq)) were dissolved in acetone and refluxed overnight to obtain Intermediate 3-1. Intermediate 3-1 was determined (confirmed) through LC-MS.

C₁₉H₇D₇BrNO₂S M+1: 407.1

### Synthesis of Intermediate 3-2

Intermediate 3-1 (1 eq) and 9H-carbazole-1,2,3,4,5,6,7,8-d8 (CAS #=38537-24-5) (1 eq) were dissolved in toluene, and refluxed overnight under the conditions of Cul (0.5 eq), ethylenediamine (2 eq), and potassium phosphate (3 eq) to obtain Intermediate 3-2. Intermediate 3-2 was determined through LC-MS.

C₃₁H₇D₁₅N₂O₂S M+1:502.3

### Synthesis of Intermediate 3-3

Intermediate 3-2 (1 eq) and KOH (5 eq) were dissolved in a solution of THF:H₂O═1:1 (volume ratio), and refluxed overnight to obtain Intermediate 3-3.

Intermediate 3-3 was determined through LC-MS.

C₂₄HD₁₅N₂ M+1: 347.8

### Synthesis of Compound 3

2.8 g of Intermediate 3-3 and 3.6 g of 2-bromo-9-phenyl-9H-carbazole (CAS #=94994-62-4) were placed in a reaction vessel, and 0.32 g of Pd₂dba₃, 0.1 g of P(tBu)₃, 1.51 g of NaOtBu, and 50 mL of toluene were added dropwise. The reaction temperature was raised to 120° C., and the mixture was refluxed for 12 hours. After the reaction was completed, a reaction solution was extracted with ethyl acetate and an organic layer was collected. The collected organic layer was dried over magnesium sulfate and a solvent was evaporated to obtain a residue. The obtained residue was separated and purified through silica gel column chromatography to obtain 3.9 g (yield: 76%) of Compound 3. Compound 3 was determined through LC-MS.

C₄₂H₁₂D₁₅N₃ M+1: 589.3

### (2) Synthesis of Heterocyclic Compound 4

Heterocyclic Compound 4 according to one or more embodiments may be synthesized by, for example, a process of Reaction Formula 2 below.

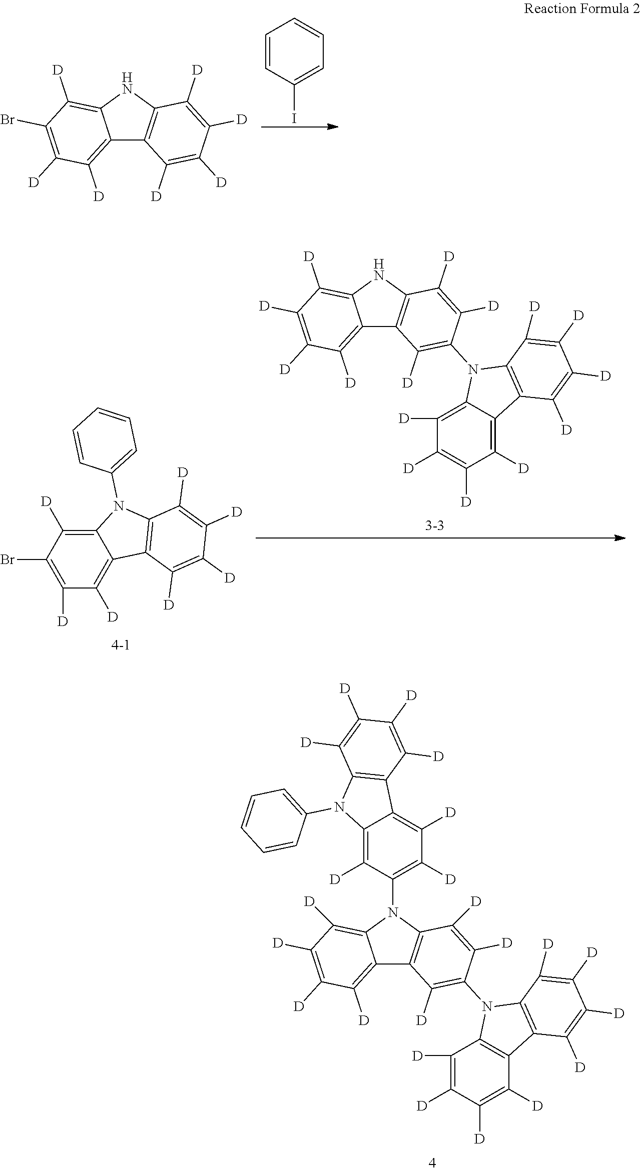 [SMILES: [2H]c1c([2H])c([2H])c2c([nH]c3c([2H])c(Br)c([2H])c([2H])c32)c1[2H] ; Ic1ccccc1 ; [2H]c1c([2H])c([2H])c2c(c1[2H])c1c([2H])c([2H])c(Br)c([2H])c1n2-c1ccccc1 ; [2H]c1c([2H])c([2H])c2c([nH]c3c([2H])c([2H])c(-n4c5c([2H])c([2H])c([2H])c([2H])c5c5c([2H])c([2H])c([2H])c([2H])c54)c([2H])c32)c1[2H] ; [2H]c1c([2H])c([2H])c2c(c1[2H])c1c([2H])c([2H])c(-n3c4c([2H])c([2H])c([2H])c([2H])c4c4c([2H])c(-n5c6c([2H])c([2H])c([2H])c([2H])c6c6c([2H])c([2H])c([2H])c([2H])c65)c([2H])c([2H])c43)c([2H])c1n2-c1ccccc1]

### Synthesis of Intermediate 4-1

2-bromo-9H-carbazole-1,3,4,5,6,7,8-d7 (CAS #=2650519-97-2), iodobenzene, Cul(0.5 eq), ethylenediamine(2 eq), and potassium phosphate(3 eq) were refluxed overnight to obtain Intermediate 4-1. Intermediate 4-1 was determined through LC-MS.

C₁₈H₅D₇BrN M+1: 329.1

### Synthesis of Compound 4

4.1 g of Intermediate 3-3 and 3.2 g of Intermediate 4-1 were placed in a reaction vessel, and 0.36 g of Pd₂dba₃, 0.1 g of P(tBu)3, 1.7 g of NaOtBu, and 30 mL of toluene were added dropwise. The reaction temperature was raised to 120° C., and the mixture was refluxed for 12 hours. After the reaction was completed, a reaction solution was extracted with ethyl acetate and an organic layer was collected. The collected organic layer was dried over magnesium sulfate and a solvent was evaporated to obtain a residue. The obtained residue was separated and purified through silica gel column chromatography to obtain 4.5 g (yield: 77%) of Compound 4. Compound 4 was determined through LC-MS.

C₄₂H₅D₂₂N₃ M+1: 595.36

### (3) Synthesis of Heterocyclic Compound 5

Heterocyclic Compound 5 according to one or more embodiments may be synthesized by, for example, a process of Reaction Formula 3 below.

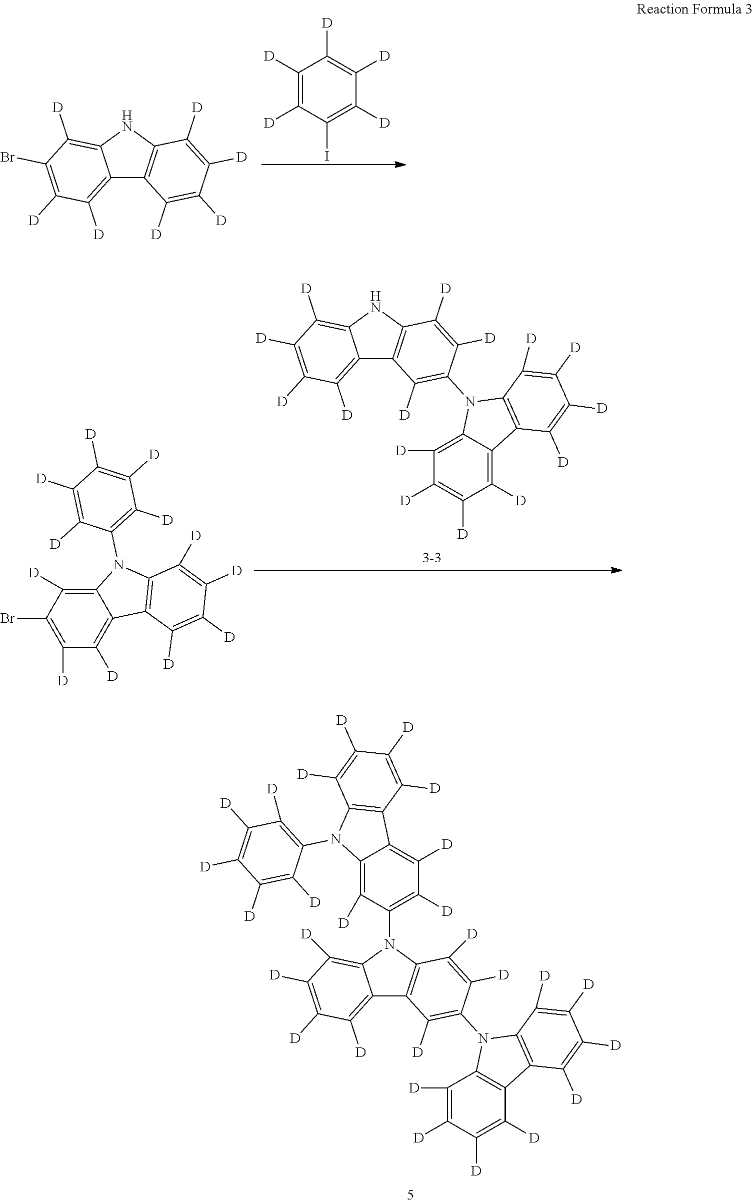

### Synthesis of Intermediate 5-1

2-bromo-9H-carbazole-1,3,4,5,6,7,8-d7(CAS #=2650519-97-2) (1 eq), 1-iodobenzene-2,3,4,5,6-d5 (CAS #=7379-67-1) (1 eq), Cul (0.5 eq), ethylenediamine (2 eq), and potassium phosphate (3 eq) were refluxed overnight to obtain Intermediate 5-1. Intermediate 5-1 was determined through LC-MS.

C₁₈D₁₂BrN M+1: 334.1

### Synthesis of Compound 5

9.7 g of Intermediate 5-1 and 10.1 g of Intermediate 3-3 were placed in a reaction vessel, and 0.12 g of Pd₂dba₃, 0.6 g of P(tBu)₃, 4.2 g of NaOtBu, and 150 mL of toluene were added dropwise. The reaction temperature was raised to 120° C., and the mixture was refluxed for 12 hours. After the reaction was completed, a reaction solution was extracted with ethyl acetate and an organic layer was collected. The collected organic layer was dried over magnesium sulfate and a solvent was evaporated to obtain a residue. The obtained residue was separated and purified through silica gel column chromatography to obtain 7.3 g (yield: 42%) of Compound 5. Compound 5 was determined through LC-MS.

C₄₂D₂₇N₃ M+1: 601.4

### (4) Synthesis of Heterocyclic Compound 8

Heterocyclic compound 8 according to one or more embodiments may be synthesized by, for example, a process of Reaction Formula 4 below.

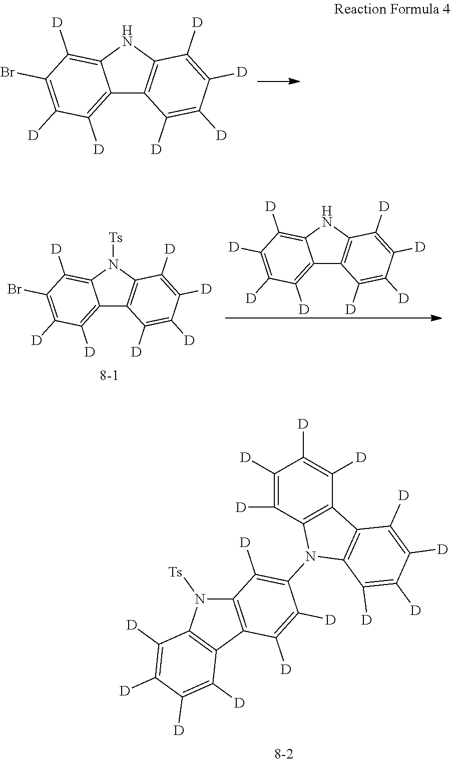

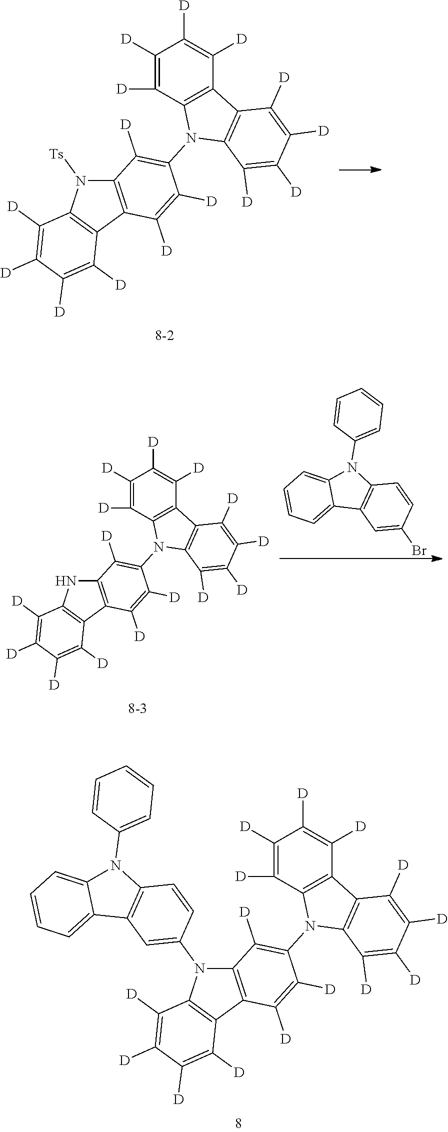

### Synthesis of Intermediate 8-1

2-bromo-9H-carbazole-1,2,4,5,6,7,8-d7(CAS #=2650519-97-2) (1 eq), TsCl (1 eq), and KOH (1 eq)) were dissolved in acetone and refluxed overnight to obtain Intermediate 8-1. Intermediate 8-1 was determined through LC-MS.

C₁₉H₇D₇BrNO₂S M+1: 406.04

### Synthesis of Intermediate 8-2

Intermediate 8-1 (1 eq) and 9H-carbazole-1,2,3,4,5,6,7,8-d8 (CAS #=38537-24-5) (1 eq) were dissolved in toluene, and refluxed overnight under the conditions of Cul (0.5 eq), ethylenediamine (2 eq), and potassium phosphate (3 eq) to obtain Intermediate 8-2. Intermediate 8-2 was determined through LC-MS.

C₃₁H₇D₁₅N₂O₂S M+1: 502.3

### Synthesis of Intermediate 8-3

Intermediate 8-2 (1 eq) and KOH (5 eq) were dissolved in a solution of THF:H₂O=1:1 (volume ratio), and refluxed overnight to obtain Intermediate 8-2. Intermediate 8-3 was determined through LC-MS.

C₂₄HD₁₅N₂ M+1: 347.23

### Synthesis of Compound 8

5.3 g of Intermediate 8-3 and 4.1 g of 3-bromo-9-phenyl-9H-carbazole (CAS #=1153-85-1) were placed in a reaction vessel, and 0.47 g of Pd₂dba₃, 0.11 g of P(tBu), 2.2 g of NaOtBu, and 30 mL of toluene were added dropwise. The reaction temperature was raised to 120° C., and the mixture was refluxed for 12 hours. After the reaction was completed, a reaction solution was extracted with ethyl acetate and an organic layer was collected. The collected organic layer was dried over magnesium sulfate and a solvent was evaporated to obtain a residue. The obtained residue was separated and purified through silica gel column chromatography to obtain 4.7 g (yield: 63%) of Compound 8. Compound 8 was determined through LC-MS.

C₄₂H₁₂D₁₅N₃ M+1: 589.3

### (5) Synthesis of Heterocyclic Compound 10

Heterocyclic compound 10 according to one or more embodiments may be synthesized by, for example, a process of Reaction Formula 5 below.

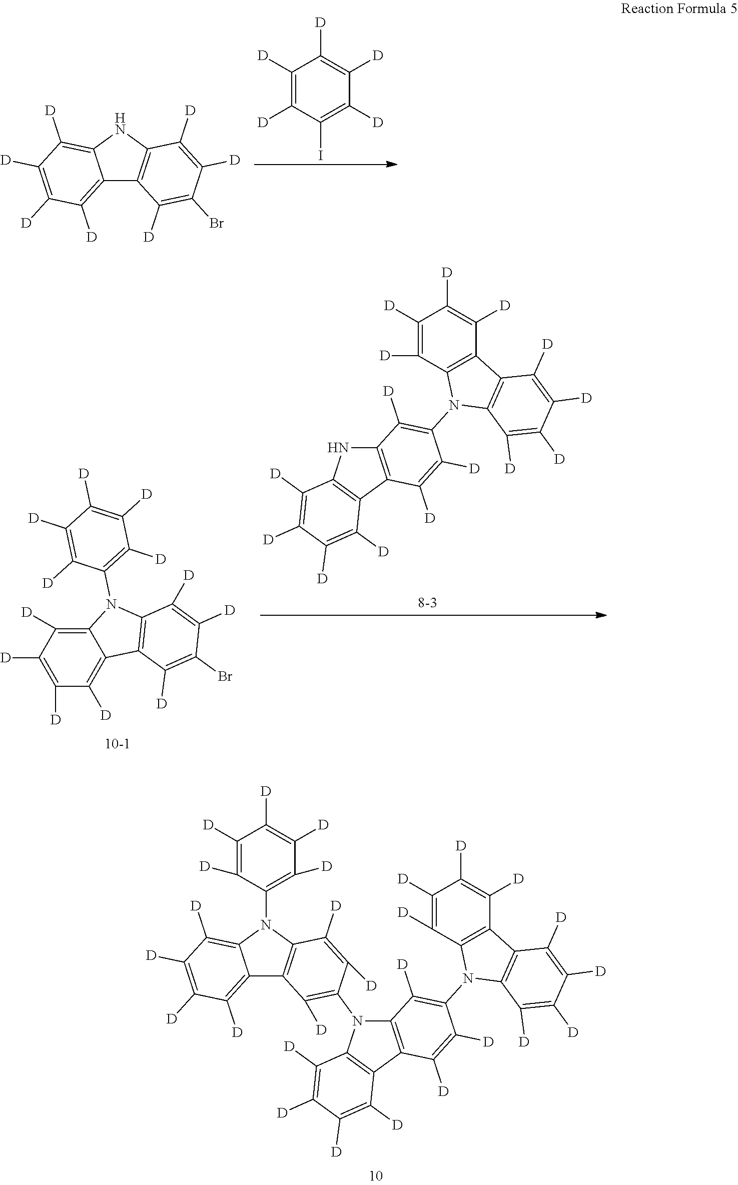

### Synthesis of Intermediate 10-1

3-bromo-9H-carbazole-1,2,4,5,6,7,8-d7(CAS #=2764814-81-3) (1 eq) and 1-iodobenzene-2,3,4,5,6-d5 (CAS #=7379-67-1) (1 eq) were dissolved in toluene and refluxed overnight under the conditions of Cul (0.5 eq), ethylenediamine (2 eq), and potassium phosphate (3 eq) to obtain Intermediate 10-1. Intermediate 10-1 was determined through LC-MS.

C₁₈D₁₂BrN M+1: 334.2

### Synthesis of Compound 10

3.1 g of Intermediate 10-1 and 3.9 g of Intermediate 8-3 were placed in a reaction vessel, and 0.34 g of Pd₂dba₃, 0.1 g of P(tBu)3, 1.6 g of NaOtBu, and 50 mL of toluene were added dropwise. The reaction temperature was raised to 120° C., and the mixture was refluxed for 12 hours. After the reaction was completed, a reaction solution was extracted with ethyl acetate and an organic layer was collected. The collected organic layer was dried over magnesium sulfate and a solvent was evaporated to obtain a residue. The obtained residue was separated and purified through silica gel column chromatography to obtain 3.3 g (yield: 59%) of Compound 10. Compound 10 was determined through LC-MS.

C₄₂D₂₇N₃ M+1: 601.3

### (6) Synthesis of Heterocyclic Compound 18

Heterocyclic compound 18 according to one or more embodiments may be synthesized by, for example, a process of Reaction Formula 6 below.

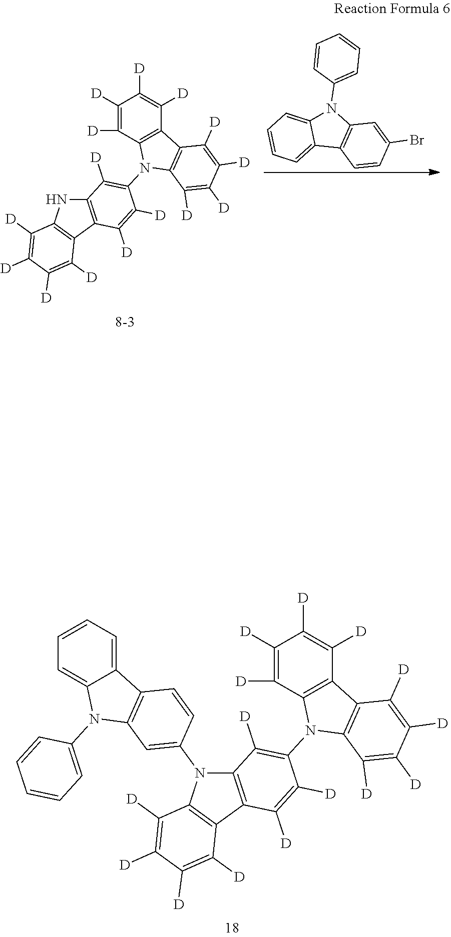

### Synthesis of Compound 18

4.8 g of Intermediate 8-3 and 3.7 g of 2-bromo-9-phenyl-9H-carbazole (CAS #=94994-62-4) were placed in a reaction vessel, and 0.42 g of Pd₂dba₃, 0.1 g of P(tBu)3, 2.0 g of NaOtBu, and 60 mL of toluene were added dropwise. The reaction temperature was raised to 120° C., and the mixture was refluxed for 12 hours. after the reaction was completed, a reaction solution was extracted with ethyl acetate, a collected organic layer was dried over magnesium sulfate, and a residue obtained after evaporating a solvent was separated and purified through silica gel column chromatography to obtain 4.3 g (yield: 64%) of Compound 18. Compound 18 was determined through LC-MS.

C₄₂H₁₂D₁₅N₃ M+1: 589.3

### (7) Synthesis of Heterocyclic Compound 20

Heterocyclic compound 20 according to one or more embodiments may be synthesized by, for example, a process of Reaction Formula 7 below.

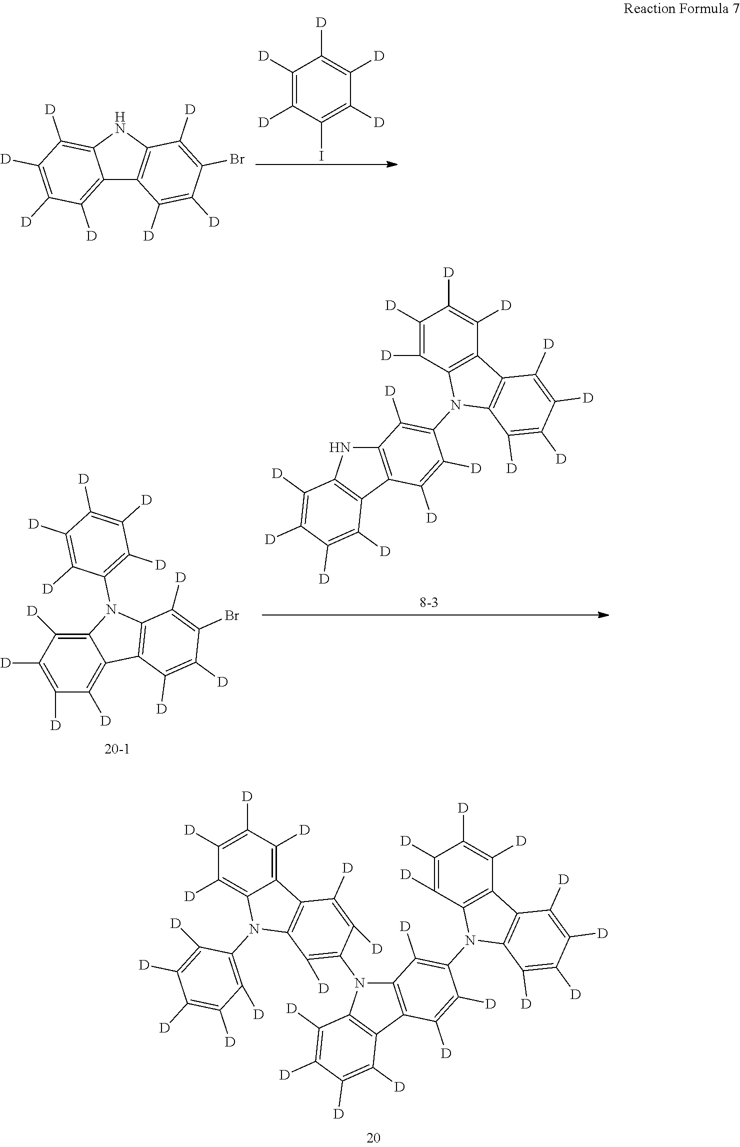

### Synthesis of Intermediate 20-1

2-bromo-9H-carbazole-1,2,4,5,6,7,8-d7(CAS #=2650519-97-2) (1 eq) and 1-iodobenzene-2,3,4,5,6-d5 (CAS #=7379-67-1) (1 eq) were dissolved in toluene and refluxed overnight under the conditions of Cul (0.5 eq), ethylenediamine (2 eq), and potassium phosphate (3 eq) to obtain Intermediate 20-1. Intermediate 20-1 was determined through LC-MS.

C₁₈D₁₂BrN M+1: 334.1

### Synthesis of Compound 20

3.2 g of Intermediate 20-1 and 4.0 g of Intermediate 8-3 were placed in a reaction vessel, and 0.35 g of Pd₂dba₃, 0.1 g of P(tBu)3, 1.7 g of NaOtBu, and 50 mL of toluene were added dropwise. The reaction temperature was raised to 120° C., and the mixture was refluxed for 12 hours. After the reaction was completed, a reaction solution was extracted with ethyl acetate and an organic layer was collected. The collected organic layer was dried over magnesium sulfate and a solvent was evaporated to obtain a residue. The obtained residue was separated and purified through silica gel column chromatography to obtain 4.2 g (yield: 73%) of Compound 20. Compound 20 was determined through LC-MS.

C₄₂D₂₇N₃ M+1: 601.3

### (8) Synthesis of Heterocyclic Compound 36

Heterocyclic compound 36 according to one or more embodiments may be synthesized by, for example, a process of Reaction Formula 8 below.

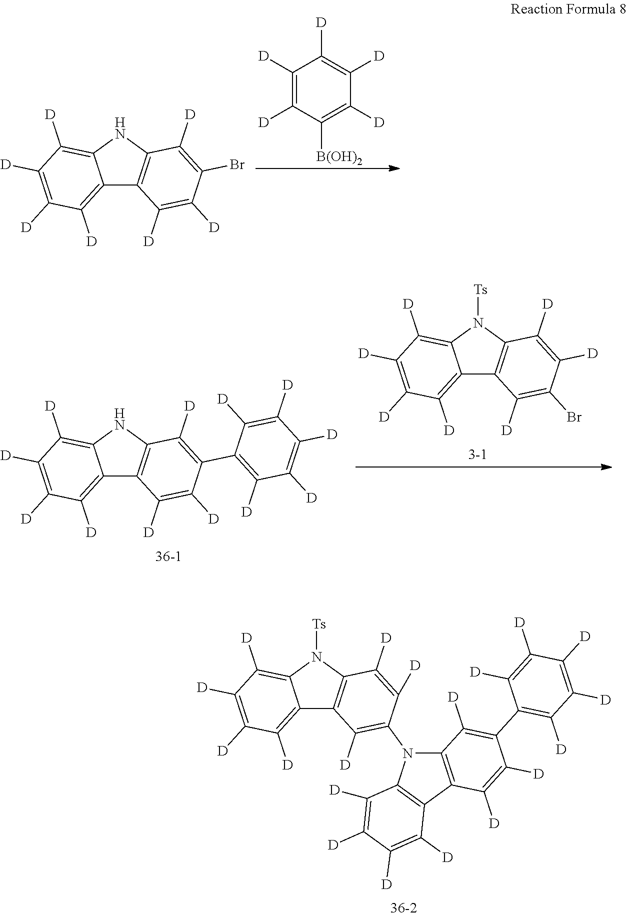 [SMILES: [2H]c1c([2H])c([2H])c2c([nH]c3c([2H])c(Br)c([2H])c([2H])c32)c1[2H] ; [2H]c1c([2H])c([2H])c(B(O)O)c([2H])c1[2H] ; [2H]c1c([2H])c([2H])c(-c2c([2H])c([2H])c3c([nH]c4c([2H])c([2H])c([2H])c([2H])c43)c2[2H])c([2H])c1[2H] ; [2H]c1c([2H])c([2H])c2c(c1[2H])c1c([2H])c(Br)c([2H])c([2H])c1n2S(=O)(=O)c1ccc(C)cc1 ; [2H]c1c([2H])c([2H])c(-c2c([2H])c([2H])c3c4c([2H])c([2H])c([2H])c([2H])c4n(-c4c([2H])c([2H])c5c(c4[2H])c4c([2H])c([2H])c([2H])c([2H])c4n5S(=O)(=O)c4ccc(C)cc4)c3c2[2H])c([2H])c1[2H]]

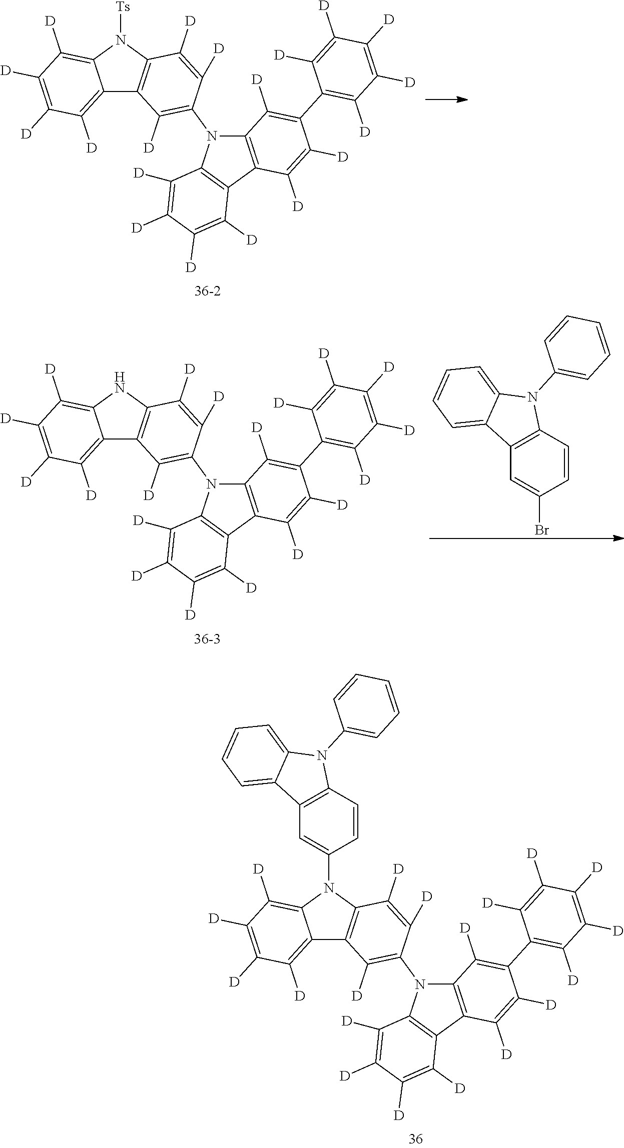

### Synthesis of Intermediate 36-1

2-bromo-9H-carbazole-1,2,4,5,6,7,8-d7 (CAS #=2650519-97-2) (1 eq), (phenyl-d5)boronic acid (1.1 eq), 0.58 g of tetrakis(triphenylphosphine)palladium (0.05 eq), and potassium carbonate (2.5 eq) were placed in a reaction vessel, dissolved in toluene, ethanol, and distilled water(a volume ratio of toluene, ethanol, and distilled water is 4d1:1), and refluxed overnight to obtain Intermediate 36-1. Intermediate 36-1 was determined through LC-MS.

C₁₈HD₁₂N M+1: 256.2

### Synthesis of Intermediate 36-2

Intermediate 36-1 (1 eq) and Intermediate 3-1 (1 eq) were dissolved in toluene, and refluxed overnight under the conditions of Cul (0.5 eq), ethylenediamine (2 eq), and potassium phosphate (3 eq) to obtain Intermediate 36-2. Intermediate 36-2 was determined through LC-MS.

C₃₇H₇D₁₉N₂O₂S M+1: 582.3

### Synthesis of Intermediate 36-3

Intermediate 36-2 (1 eq) and KOH (5 eq) were dissolved in a solution of THF:H₂O=1:1 (volume ratio), and refluxed overnight to obtain Intermediate 36-3. Intermediate 36-3 was determined through LC-MS.

C₃₀HD₁₉N₂ M+1: 428.3

### Synthesis of Compound 36

6.9 g of Intermediate 36-3 and 4.3 g of 3-bromo-9-phenyl-9H-carbazole (CAS #=1153-85-1) were placed in a reaction vessel, and 0.50 g of Pd₂dba₃, 0.12 g of P(tBu)3, 2.3 g of NaOtBu, and 70 mL of toluene were added dropwise. The reaction temperature was raised to 120° C., and the mixture was refluxed for 12 hours. After the reaction was completed, a reaction solution was extracted with ethyl acetate and an organic layer was collected. The collected organic layer was dried over magnesium sulfate and a solvent was evaporated to obtain a residue. The obtained residue was separated and purified through silica gel column chromatography to obtain 5.5 g (yield: 61%) of Compound 36. Compound 36 was determined through LC-MS.

C₄₈H₁₂D₁₉N₃ M+1: 669.4

### (9) Synthesis of Heterocyclic Compound 39

Heterocyclic Compound 39 according to one or more embodiments may be synthesized by, for example, a process of Reaction Formula 9 below.

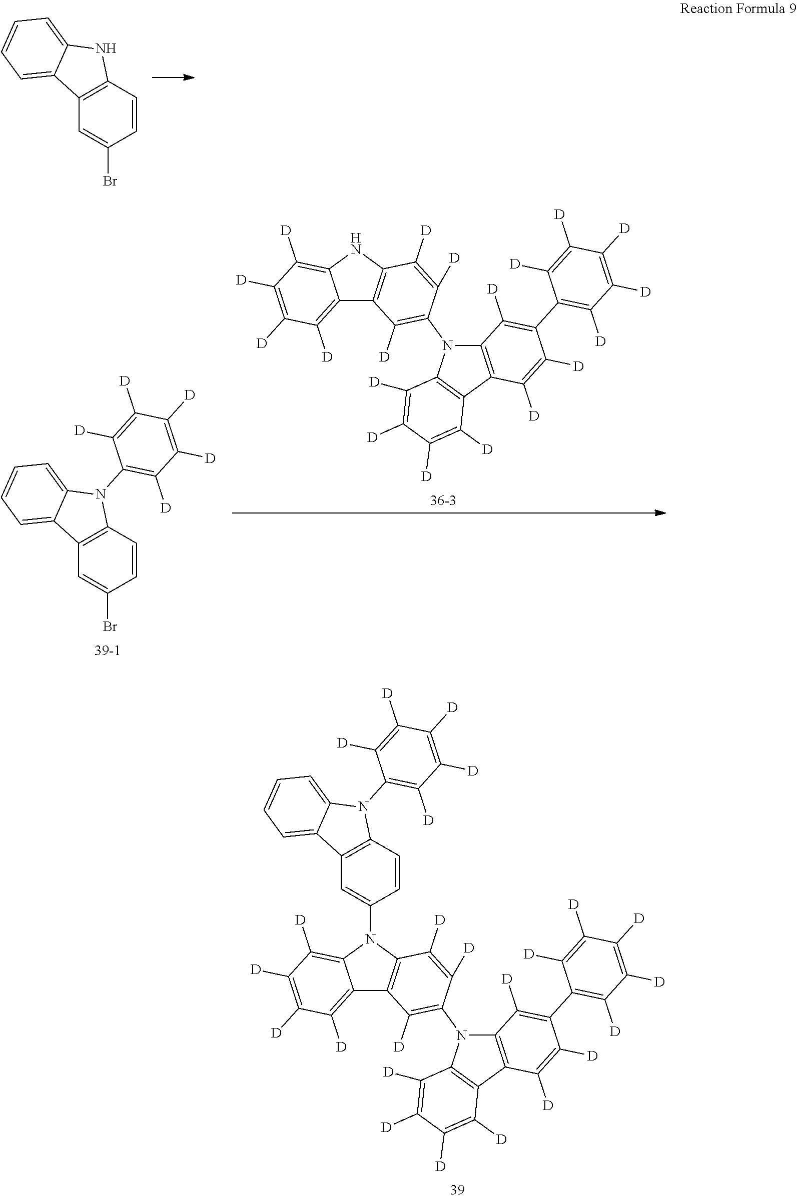 [SMILES: Brc1ccc2[nH]c3ccccc3c2c1 ; [2H]c1c([2H])c([2H])c(-n2c3ccccc3c3cc(Br)ccc32)c([2H])c1[2H] ; [2H]c1c([2H])c([2H])c(-c2c([2H])c([2H])c3c4c([2H])c([2H])c([2H])c([2H])c4n(-c4c([2H])c([2H])c5[nH]c6c([2H])c([2H])c([2H])c([2H])c6c5c4[2H])c3c2[2H])c([2H])c1[2H] ; [2H]c1c([2H])c([2H])c(-c2c([2H])c([2H])c3c4c([2H])c([2H])c([2H])c([2H])c4n(-c4c([2H])c([2H])c5c(c4[2H])c4c([2H])c([2H])c([2H])c([2H])c4n5-c4ccc5c(c4)c4ccccc4n5-c4c([2H])c([2H])c([2H])c([2H])c4[2H])c3c2[2H])c([2H])c1[2H]]

### Synthesis of Intermediate 39-1

3-bromo-9-phenyl-9H-carbazole(CAS #=1153-85-1) (1 eq) and 1-iodobenzene-2,3,4,5,6-d5 (CAS #=7379-67-1) (1 eq) were dissolved in toluene and refluxed overnight under the conditions of Cul (0.5 eq), ethylenediamine (2 eq), and potassium phosphate (3 eq) to obtain Intermediate 39-1. Intermediate 39-1 was determined through LC-MS.

C₁₈H₇D₅BrN M+1: 327.1

### Synthesis of Compound 39

4.4 g of Intermediate 39-1 and 6.9 g of Intermediate 36-3 were placed in a reaction vessel, and 0.50 g of Pd₂dba₃, 0.12 g of P(tBu)3, 2.3 g of NaOtBu, and 30 mL of toluene were added dropwise. The reaction temperature was raised to 120° C., and the mixture was refluxed for 12 hours. After the reaction was completed, a reaction solution was extracted with ethyl acetate and an organic layer was collected. The collected organic layer was dried over magnesium sulfate and a solvent was evaporated to obtain a residue. The obtained residue was separated and purified through silica gel column chromatography to obtain 5.5 g (yield: 60%) of Compound 39. Compound 39 was determined through LC-MS.

C₄₈H₇D₂₄N₃ M+1: 674.5

### 2. Preparation and Evaluation of Light Emitting Elements

### (1) Preparation of Light Emitting Elements

Light emitting elements including heterocyclic compounds according to one or more embodiments or Comparative Example compounds were prepared through a method below. Light emitting elements of Examples were prepared utilizing compounds 3, 4, 5, 8, 10, 18, 20, 36, and 39, which are heterocyclic compounds of one or more embodiments, as host materials for emission layers. Light emitting elements of Comparative Examples 1 to 9 were prepared utilizing Comparative Example Compounds CX1 to CX9 as host materials for emission layers.

As an anode, an ITO glass substrate (corning, 15 Ω/cm², 1200 Å) was cut to a size of 50 mm×50 mm×0.5 mm, subjected to ultrasonic cleaning using isopropyl alcohol and pure water for 5 minutes respectively and ultraviolet irradiation for 30 minutes, and then exposed to ozone for cleaning to form the glass substrate in a vacuum deposition apparatus.

On the substrate, HATCN was formed to have a thickness of 100 Å. Thereafter, BCFN was vacuum-deposited to have a thickness of 600 Å to form a first hole transport layer, and SiCzCz was vacuum-deposited to have a thickness of 50 Å to form a second hole transport layer.

On the second hole transport layer, a first host material, a second host material, and a phosphorescent dopant material were co-deposited in a weight ratio of 60:27:13 to form an emission layer having a thickness of 350 Å. Example compounds or Comparative Example Compounds were provided as first host materials, SiTrzCz2 was provided as a second host material, and PtON-TBBI was provided as a phosphorescent dopant material.

mSiTrz was deposited to have a thickness of 50 Å as a first electron transport layer on the emission layer, mSiTrz and LiQ were co-deposited in a weight ratio of 1:1 to have a thickness of 350 Å as a second electron transport layer. An alkali metal halide, LiF, was deposited on the second electron transport layer to have a thickness of 15 Å as an electron injection layer, and Al was vacuum-deposited to have a thickness of 80 Å to form a LiF/Al electrode.

Materials Used Upon Preparation of Light Emitting Elements

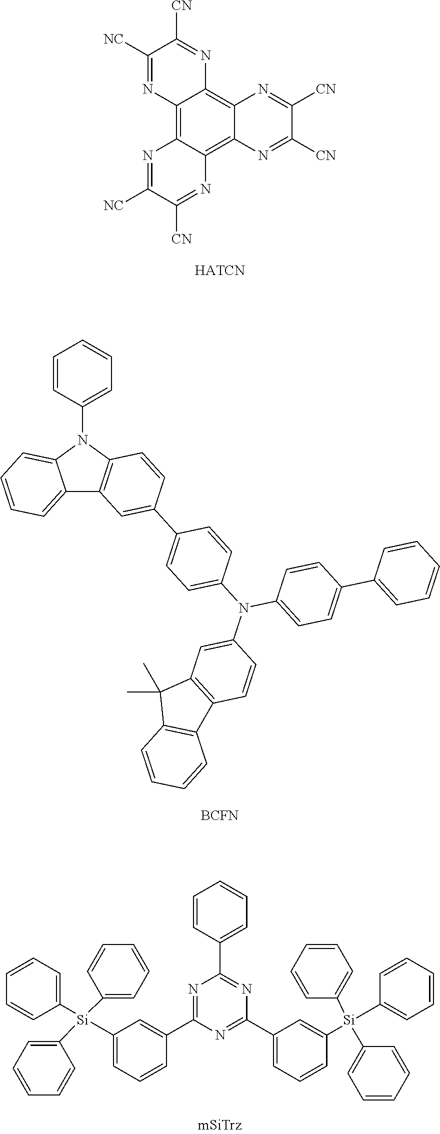

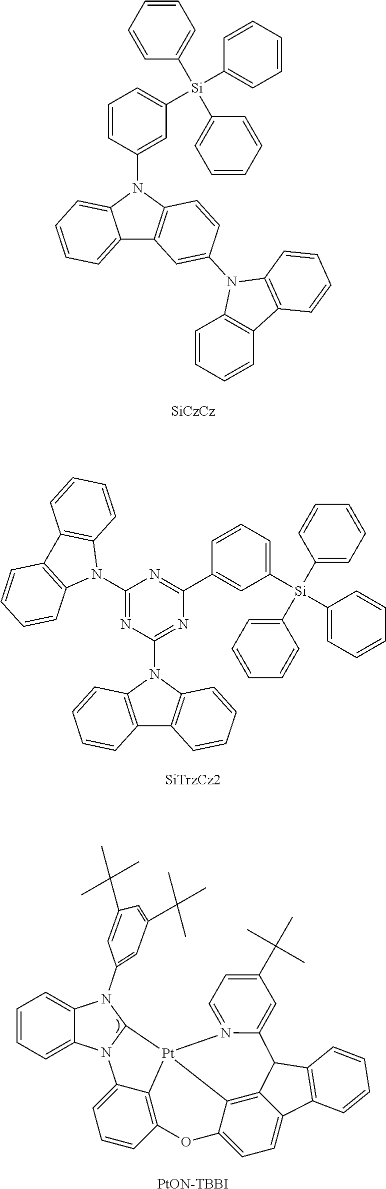

BCFN is the same as H-1-1 of Compound Group H described above. miSiTrz is the same as ETH2 of Compound Group FT described above. SiTrzCz2 is the same as ETH66 of Compound Group FT described above. PtON-TBBI is the same as AD-39 of Compound Group AD described above.

Example Compounds and Comparative Example Compounds used in the preparation of the light emitting elements of Examples and Comparative Examples are shown in Table 1 below.

TABLE 1

| | |
|---|---|
| Compound 3 | 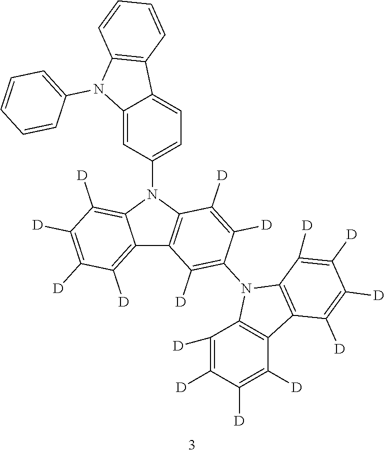 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc2c3ccccc3n(-c3ccccc3)c2c1] |
| Compound 4 | 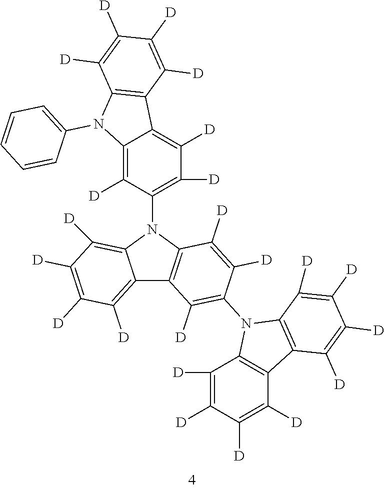 [SMILES: [2H]c1c([2H])c([2H])c2c(c1[2H])c1c([2H])c([2H])c(-n3c4c([2H])c([2H])c([2H])c([2H])c4c4c([2H])c(-n5c6c([2H])c([2H])c([2H])c([2H])c6c6c([2H])c([2H])c([2H])c([2H])c65)c([2H])c([2H])c43)c([2H])c1n2-c1ccccc1] |
| Compound 5 | 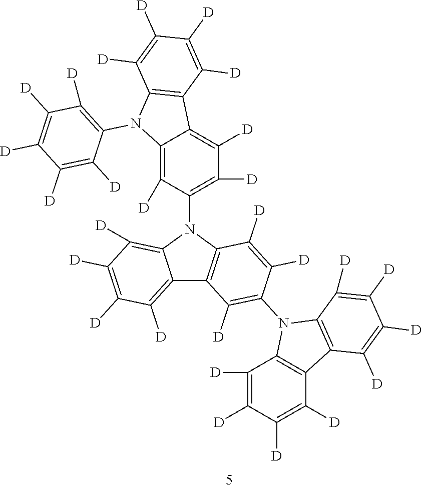 [SMILES: [2H]c1c([2H])c([2H])c(-n2c3c([2H])c([2H])c([2H])c([2H])c3c3c([2H])c([2H])c(-n4c5c([2H])c([2H])c([2H])c([2H])c5c5c([2H])c(-n6c7c([2H])c([2H])c([2H])c([2H])c7c7c([2H])c([2H])c([2H])c([2H])c76)c([2H])c([2H])c54)c([2H])c32)c([2H])c1[2H]] |
| Compound 8 | 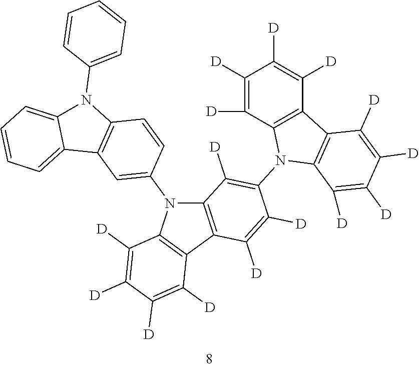 [SMILES: [2H]c1c([2H])c([2H])c2c(c1[2H])c1c([2H])c([2H])c(-n3c4c([2H])c([2H])c([2H])c([2H])c4c4c([2H])c([2H])c([2H])c([2H])c43)c([2H])c1n2-c1ccc2c(c1)c1ccccc1n2-c1ccccc1] |
| Compound 10 | 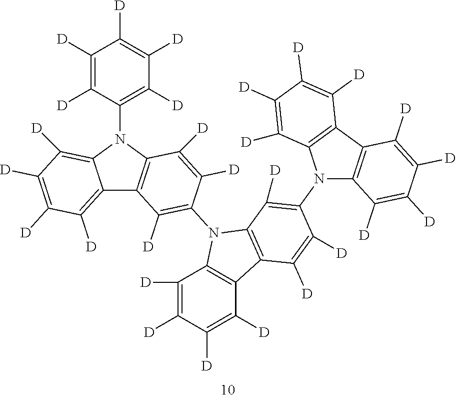 [SMILES: [2H]c1c([2H])c([2H])c(-n2c3c([2H])c([2H])c([2H])c([2H])c3c3c([2H])c(-n4c5c([2H])c([2H])c([2H])c([2H])c5c5c([2H])c([2H])c(-n6c7c([2H])c([2H])c([2H])c([2H])c7c7c([2H])c([2H])c([2H])c([2H])c76)c([2H])c54)c([2H])c([2H])c32)c([2H])c1[2H]] |
| Compound 18 | 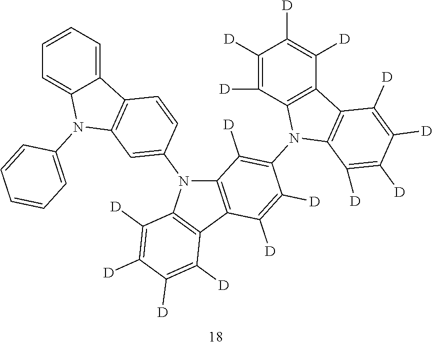 [SMILES: [2H]c1c([2H])c([2H])c2c(c1[2H])c1c([2H])c([2H])c(-n3c4c([2H])c([2H])c([2H])c([2H])c4c4c([2H])c([2H])c([2H])c([2H])c43)c([2H])c1n2-c1ccc2c3ccccc3n(-c3ccccc3)c2c1] |
| Compound 20 | 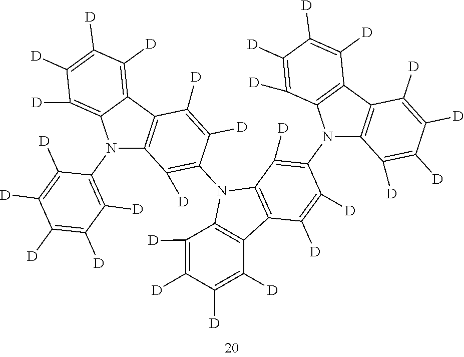 [SMILES: [2H]c1c([2H])c([2H])c(-n2c3c([2H])c([2H])c([2H])c([2H])c3c3c([2H])c([2H])c(-n4c5c([2H])c([2H])c([2H])c([2H])c5c5c([2H])c([2H])c(-n6c7c([2H])c([2H])c([2H])c([2H])c7c7c([2H])c([2H])c([2H])c([2H])c76)c([2H])c54)c([2H])c32)c([2H])c1[2H]] |
| Compound 36 | 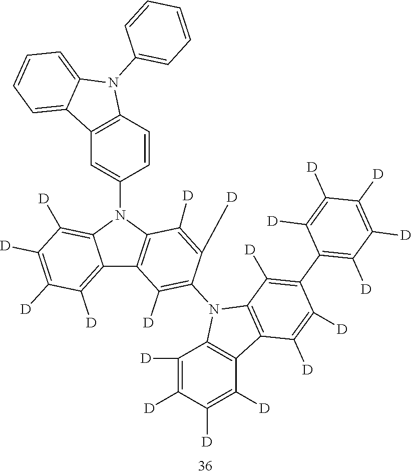 [SMILES: [2H]c1c([2H])c([2H])c(-c2c([2H])c([2H])c3c4c([2H])c([2H])c([2H])c([2H])c4n(-c4c([2H])c([2H])c5c(c4[2H])c4c([2H])c([2H])c([2H])c([2H])c4n5-c4ccc5c(c4)c4ccccc4n5-c4ccccc4)c3c2[2H])c([2H])c1[2H]] |
| Compound 39 | 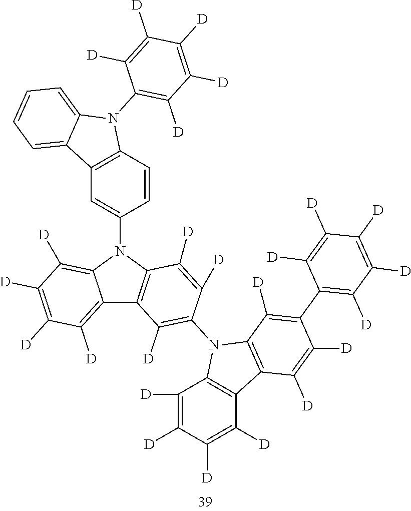 [SMILES: [2H]c1c([2H])c([2H])c(-c2c([2H])c([2H])c3c4c([2H])c([2H])c([2H])c([2H])c4n(-c4c([2H])c([2H])c5c(c4[2H])c4c([2H])c([2H])c([2H])c([2H])c4n5-c4ccc5c(c4)c4ccccc4n5-c4c([2H])c([2H])c([2H])c([2H])c4[2H])c3c2[2H])c([2H])c1[2H]] |
| — | — |
| Comparative Example Compound CX1 | 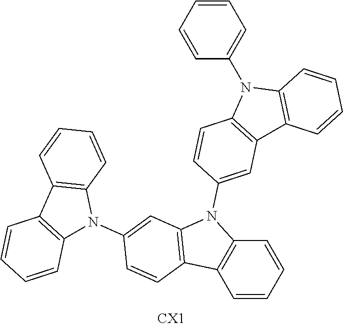 [SMILES: c1ccc(-n2c3ccccc3c3cc(-n4c5ccccc5c5ccc(-n6c7ccccc7c7ccccc76)cc54)ccc32)cc1] |
| Comparative Example Compound CX2 | 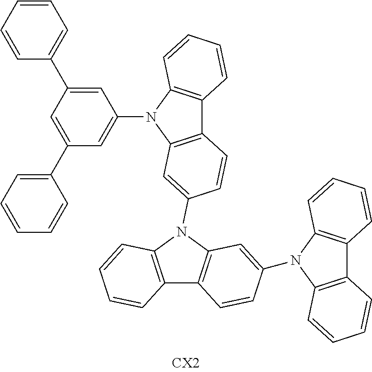 [SMILES: c1ccc(-c2cc(-c3ccccc3)cc(-n3c4ccccc4c4ccc(-n5c6ccccc6c6ccc(-n7c8ccccc8c8ccccc87)cc65)cc43)c2)cc1] |
| Comparative Example Compound CX3 | 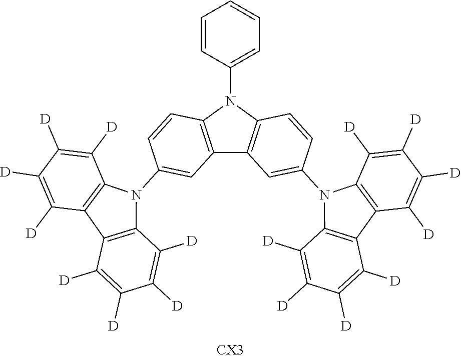 [SMILES: [2H]c1c([2H])c([2H])c2c(c1[2H])c1c([2H])c([2H])c([2H])c([2H])c1n2-c1ccc2c(c1)c1cc(-n3c4c([2H])c([2H])c([2H])c([2H])c4c4c([2H])c([2H])c([2H])c([2H])c43)ccc1n2-c1ccccc1] |
| Comparative Example Compound CX4 | 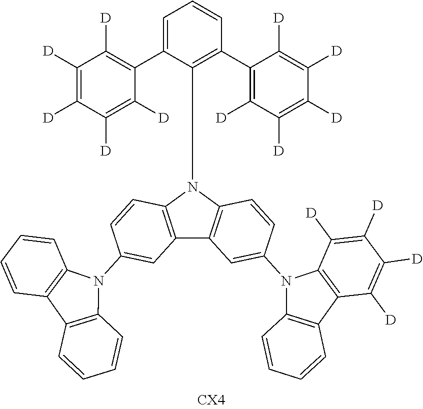 [SMILES: [2H]c1c([2H])c([2H])c(-c2cccc(-c3c([2H])c([2H])c([2H])c([2H])c3[2H])c2-n2c3ccc(-n4c5ccccc5c5ccccc54)cc3c3cc(-n4c5ccccc5c5c([2H])c([2H])c([2H])c([2H])c54)ccc32)c([2H])c1[2H]] |
| Comparative Example Compound CX5 | 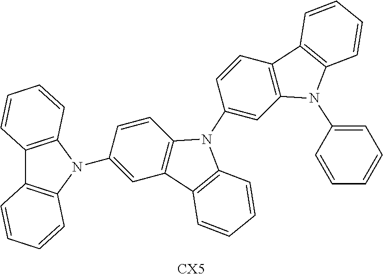 [SMILES: c1ccc(-n2c3ccccc3c3ccc(-n4c5ccccc5c5cc(-n6c7ccccc7c7ccccc76)ccc54)cc32)cc1] |
| Comparative Example Compound CX6 | 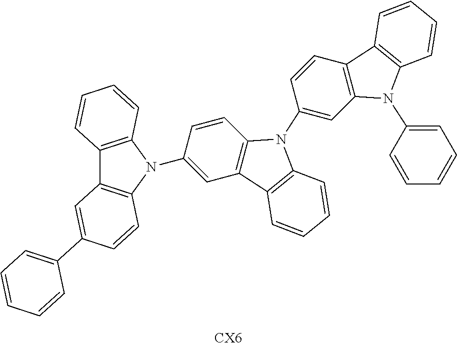 [SMILES: c1ccc(-c2ccc3c(c2)c2ccccc2n3-c2ccc3c(c2)c2ccccc2n3-c2ccc3c4ccccc4n(-c4ccccc4)c3c2)cc1] |
| Comparative Example Compound CX7 | 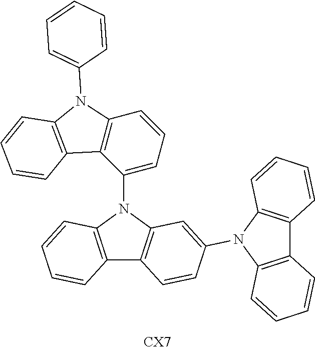 [SMILES: c1ccc(-n2c3ccccc3c3c(-n4c5ccccc5c5ccc(-n6c7ccccc7c7ccccc76)cc54)cccc32)cc1] |
| Comparative Example Compound CX8 | 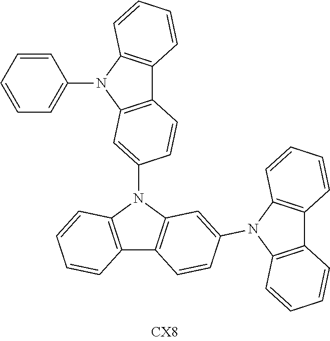 [SMILES: c1ccc(-n2c3ccccc3c3ccc(-n4c5ccccc5c5ccc(-n6c7ccccc7c7ccccc76)cc54)cc32)cc1] |
| Comparative Example Compound CX9 | 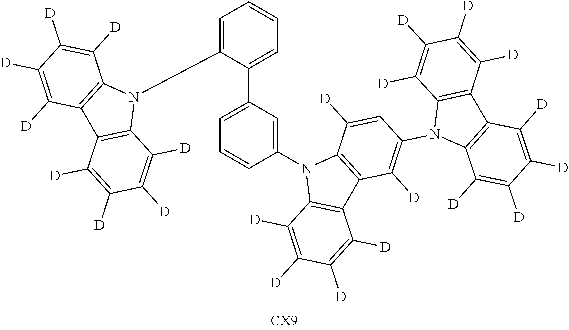 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)cc([2H])c1n2-c1cccc(-c2ccccc2-n2c3c([2H])c([2H])c([2H])c([2H])c3c3c([2H])c([2H])c([2H])c([2H])c32)c1] |
| — | — |

### (2) Evaluation of Light Emitting Elements

Table 2 below shows evaluation results of the light emitting elements of Examples and Comparative Examples. Driving voltage and maximum quantum efficiency at a current density of 10 mA/cm² were measured and are shown in Table 2 below. The driving voltage of a light emitting element was determined using a source meter (2400 series from Keithley Instruments), and the maximum quantum efficiency was determined using an external quantum efficiency measuring apparatus (C9920-2-12 from Hamamatsu Photonics) In the evaluation of the maximum quantum efficiency, the luminance and current densities were measured using a luminance meter that was calibrated for wavelength sensitivity, and the maximum quantum efficiency was converted under the assumption that an angular luminance distribution (Lambertian) was obtained with respect to a fully diffused reflective surface. Time taken for luminance to reach 95% with respect to an initial luminance was measured as lifespan, and relative lifespan was calculated with respect to Example 1, and the results are shown.

TABLE 2

| Example | | | Maximum | | |
|---|---|---|---|---|---|
| of | | Driving | quantum | | |
| element | Host | voltage | efficiency | Lifespan | Emitted |
| preparation | material | (V) | (%) | (%) | color |
| Example 1 | Compound 3 | 5.3 | 22.2 | 100 | Blue |
| Example 2 | Compound 4 | 5.3 | 22.4 | 133 | Blue |
| Example 3 | Compound 5 | 5.1 | 23.4 | 129 | Blue |
| Example 4 | Compound 8 | 5.3 | 24.1 | 115 | Blue |
| Example 5 | Compound 10 | 5.1 | 23.8 | 113 | Blue |
| Example 6 | Compound 18 | 5.3 | 22.7 | 112 | Blue |
| Example 7 | Compound 20 | 5.4 | 22.8 | 119 | Blue |
| Example 8 | Compound 36 | 5.2 | 23.5 | 109 | Blue |
| Example 9 | Compound 39 | 5.1 | 23.6 | 118 | Blue |
| Comparative | Comparative | 5.5 | 21.8 | 61 | Blue |
| Example 1 | Example | | | | |
| | Compound | | | | |
| | CX1 | | | | |
| Comparative | Comparative | 5.6 | 19.2 | 47 | Blue |
| Example 2 | Example | | | | |
| | Compound | | | | |
| | CX2 | | | | |
| Comparative | Comparative | 5.5 | 22.2 | 44 | Blue |
| Example 3 | Example | | | | |
| | Compound | | | | |
| | CX3 | | | | |
| Comparative | Comparative | 5.6 | 21.3 | 51 | Blue |
| Example 4 | Example | | | | |
| | Compound | | | | |
| | CX4 | | | | |
| Comparative | Comparative | 5.4 | 23.3 | 62 | Blue |
| Example 5 | Example | | | | |
| | Compound | | | | |
| | CX5 | | | | |
| Comparative | Comparative | 5.5 | 23.4 | 55 | Blue |
| Example 6 | Example | | | | |
| | Compound | | | | |
| | CX6 | | | | |
| Comparative | Comparative | 5.8 | 22.1 | 38 | Blue |
| Example 7 | Example | | | | |
| | Compound | | | | |
| | CX7 | | | | |
| Comparative | Comparative | 5.6 | 22.6 | 61 | Blue |
| Example 8 | Example | | | | |
| | Compound | | | | |
| | CX8 | | | | |
| Comparative | Comparative | 5.9 | 21.0 | 70 | Blue |
| Example 9 | Example | | | | |
| | Compound | | | | |
| | CX9 | | | | |

Referring to Table 2, it is seen that, compared to the light emitting elements of Comparative Examples 1 to 9, the light emitting elements of Examples 1 to 9 exhibit significantly longer service life. In some embodiments, it is seen that, compared to the light emitting element of Comparative Example 1 to 9, the light emitting elements of Examples 1 to 9 have reduced driving voltage. It is seen that the light emitting elements of Examples 4, 5, 8, and 9 exhibit excellent (improved) maximum quantum efficiency.

The light emitting elements of Examples 1 to 9 include Compounds 3, 4, 5, 8, 10, 18, 20, 36, and 39, and Compounds 3, 4, 5, 8, 10, 18, 20, 36, and 39 are heterocyclic compounds of one or more embodiments. Compounds 3, 4, 5, 8, 10, 18, 20, 36, and 39 include three carbazole groups (i.e., first to third carbazole groups) and a deuterium atom directly bonded to at least one selected from among the first to third carbazole groups. Accordingly, the heterocyclic compound of one or more embodiments may contribute to increasing lifespan.

The light emitting element of Comparative Example 1 includes Comparative Example Compound CX1, and the light emitting element of Comparative Example 2 includes Comparative Example Compound CX2. Comparative Example Compounds CX1 and CX2 include three carbazole groups, but do not include deuterium atoms. It is believed that, at least in part due to this reason, the light emitting elements of Comparative Examples 1 and 2 exhibit relatively short lifespan.

The light emitting element of Comparative Example 3 includes Comparative Example Compound CX3, and the light emitting element of Comparative Example 4 includes Comparative Example Compound CX4. Comparative Examples Compounds CX3 and CX4 include three carbazole groups, but the second carbazole group and the third carbazole group are both bonded to the first carbazole group. In addition, Comparative Example Compounds CX3 and CX4 do not contain deuterium atoms. It is believed that, at least in part due to these reasons, the light emitting elements of Comparative Examples 3 and 4 exhibit relatively short lifespan.

The light emitting element of Comparative Example 5 includes Comparative Example Compound CX5, and the light emitting element of Comparative Example 6 includes Comparative Example Compound CX6. The light emitting element of Comparative Example 7 includes Comparative Example Compound CX7, and the light emitting element of Comparative Example 8 includes Comparative Example Compound CX8. Comparative Example Compounds CX5 to CX8 include three carbazole groups, but do not include deuterium atoms. It is believed that, at least in part due to this reason, the light emitting elements of Comparative Examples 5 to 8 exhibit relatively short lifespan.

The light emitting element of Comparative Example 9 includes Comparative Example Compound CX9. Comparative Examples Compound CX9 includes three carbazole groups and a deuterium atom, but the second carbazole group and the third carbazole group are both bonded to the first carbazole group. Any one of the second carbazole group and the third carbazole group is directly bonded to the first carbazole group, and the other is indirectly bonded to the first carbazole group through a phenyl group. It is believed that, at least in part due to this reason, the light emitting element of Comparative Example 9 exhibit relatively short lifespan.

The light emitting element of one or more embodiments may include at least one functional layer provided between a first electrode and a second electrode, and the least one functional layer may include the heterocyclic compound of one or more embodiments. The heterocyclic compound of one or more embodiments may include three carbazole groups (i.e., first to third carbazole groups), and at least one carbazole group selected from among the three carbazole groups may be substituted with a deuterium atom. Deuterium atoms may be directly bonded to the carbazole group and may be present in two or more. Accordingly, the heterocyclic compound of one or more embodiments may contribute to increasing lifespan. The light emitting element of one or more embodiments including the heterocyclic compound of one or more embodiments may exhibit long lifespan characteristics.

A light emitting element of one or more embodiments includes the heterocyclic compound of one or more embodiments, and may thus exhibit reduced driving voltage and long lifespan.

The heterocyclic compound of one or more embodiments may contribute to reduction in driving voltage and an increase in lifespan of a light emitting element.

Although the present disclosure has been described with reference to example embodiments of the present disclosure, it will be understood that the present disclosure should not be limited to these embodiments but that various changes and modifications can be made by those skilled in the art without departing from the spirit and scope of the present disclosure.

Accordingly, the technical scope of the present disclosure is not intended to be limited to the contents set forth in the detailed description of the specification, but is intended to be defined by the appended claims and their equivalents.

What is claimed is:

1. A light emitting element comprising:
a first electrode;
a second electrode on the first electrode; and
at least one functional layer between the first electrode and the second electrode, the at least one functional layer comprising a heterocyclic compound represented by Formula 1: 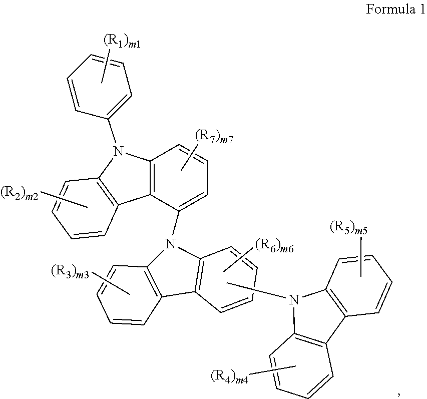
wherein in Formula 1,
m1 is an integer of 1 to 5,
m2 to m5 are each independently an integer of 1 to 4,
m6 and m7 are each independently an integer of 1 to 3,
at least one selected from among R₁ to R₇ is a deuterium atom, and
remaining ones of R₁ to R₇ are each independently a hydrogen atom or a substituted or unsubstituted aryl group having 6 to 30 ring-forming carbon atoms.

2. The light emitting element of claim 1, wherein Formula 1 is represented by any one selected from among Formulae 1-1 to 1-3: 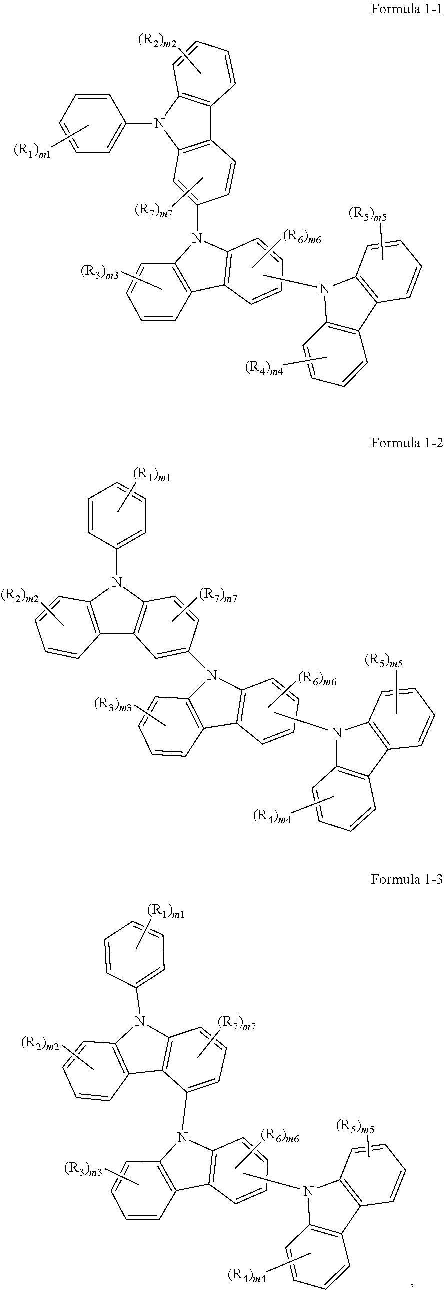
and
wherein in Formulae 1-1 to 1-3,
m1 to m7 and R₁ to R₇ are the same as defined in Formula 1.

3. The light emitting element of claim 2, wherein Formula 1-1 is represented by any one selected from among Formulae 1-1A to 1-1C: 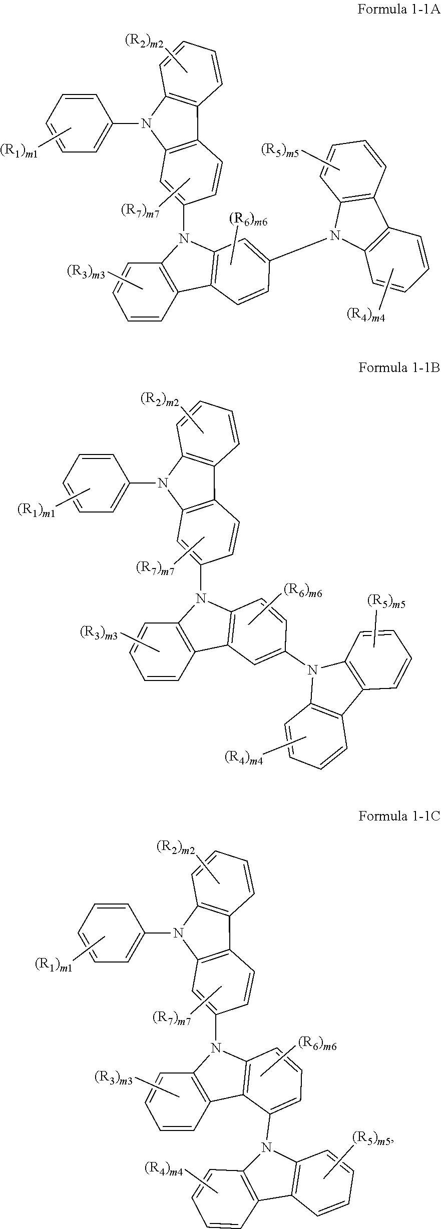
and
wherein in Formulae 1-1A to 1-1C,
m1 to m7 and R₁ to R₇ are the same as defined in Formula 1-1.

4. The light emitting element of claim 2, wherein Formula 1-2 is represented by Formula 1-2A or Formula 1-2B: 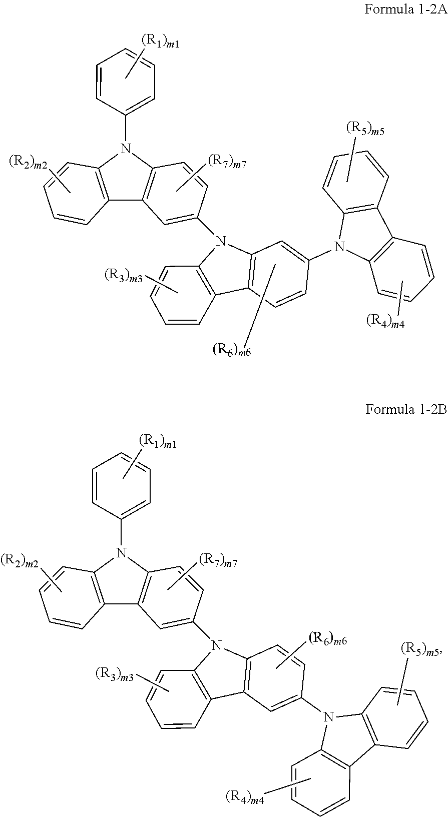
and
wherein in Formulae 1-2A and 1-2B,
m1 to m7 and R₁ to R₇ are the same as defined in Formula 1-2.

5. The light emitting element of claim 2, wherein Formula 1-3 is represented by Formula 1-3A or Formula 1-3B: 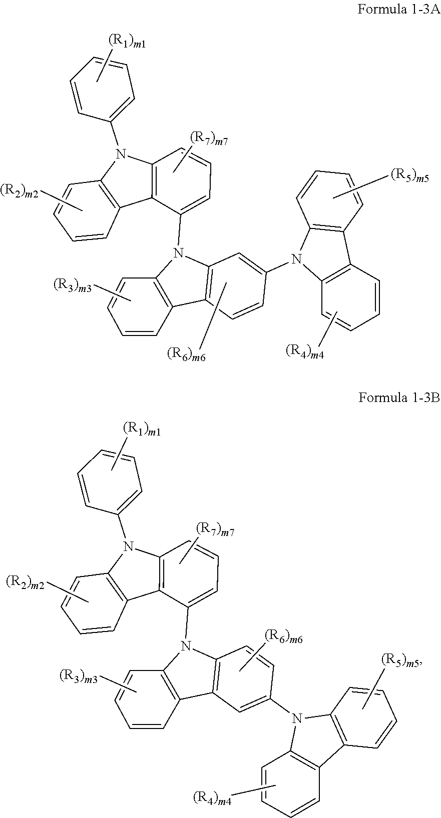
and
wherein in Formulae 1-3A and 1-3B,
m1 to m7 and R₁ to R₇ are the same as defined in Formula 1-3.

6. The light emitting element of claim 1, wherein Formula 1 is represented by Formula 1-X1 or Formula 1-X2: 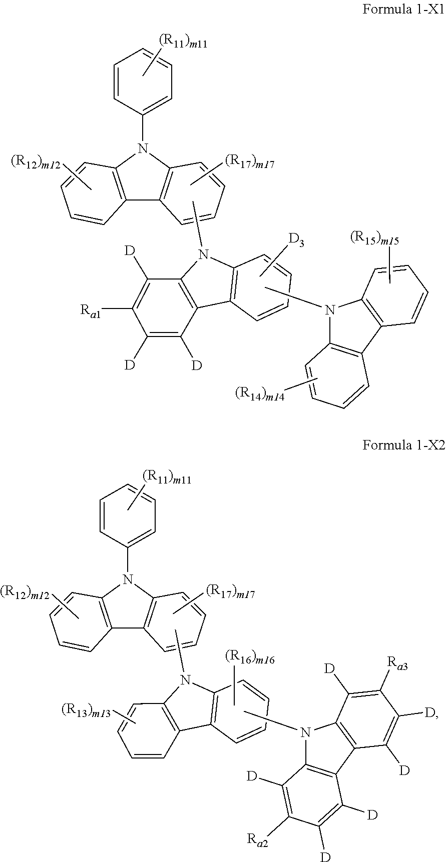
wherein in Formula 1-X1,
Rₐ₁ is a deuterium atom or a substituted or unsubstituted phenyl group,
m14 and m15 are each independently an integer of 1 to 4, and
R₁₄ and R₁₅ are each independently a hydrogen atom, a deuterium atom, or a substituted or unsubstituted phenyl group,
wherein in Formula 1-X2 above,
Rₐ₂ and Rₐ₃ are each independently a deuterium atom or a substituted or unsubstituted phenyl group,
m13 is an integer of 1 to 4,
m16 is an integer of 1 to 3, and
R₁₃ and R₁₆ are each independently a hydrogen atom, a deuterium atom, or a substituted or unsubstituted phenyl group, and
wherein in Formulae 1-X1 and 1-X2,
D is a deuterium atom,
m11 is an integer of 1 to 5,
m12 is an integer of 1 to 4,
m17 is an integer of 1 to 3, and
R₁₁, R₁₂, and R₁₇ are each independently a hydrogen atom, a deuterium atom, or a substituted or unsubstituted phenyl group.

7. The light emitting element of claim 1, wherein Formula 1 is represented by Formula 1-X3: 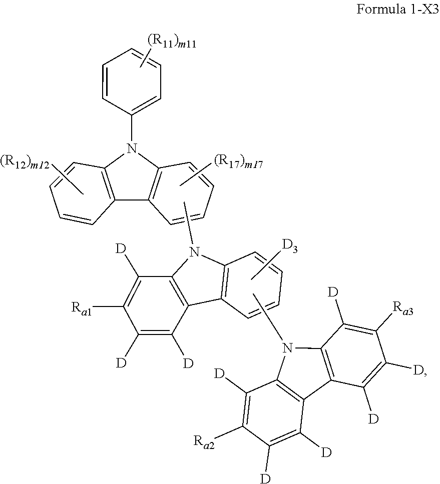
wherein in Formula 1-X3,
D is a deuterium atom,
m11 is an integer of 1 to 5,
m12 is an integer of 1 to 4,
m17 is an integer of 1 to 3,
R₁₁, R₁₂, and R₁₇ are each independently a hydrogen atom, a deuterium atom, or a substituted or unsubstituted phenyl group, and
Rₐ₁ to Rₐ₃ are each independently a deuterium atom or a substituted or unsubstituted phenyl group.

8. The light emitting element of claim 1, wherein the at least one functional layer comprises an emission layer, a hole transport region between the first electrode and the emission layer, and an electron transport region between the emission layer and the second electrode, and
at least one of the emission layer or the hole transport region comprises the heterocyclic compound.

9. The light emitting element of claim 8, wherein the emission layer comprises a dopant and a host, and
the host comprises the heterocyclic compound.

10. The light emitting element of claim 8, wherein the hole transport region comprises a hole injection layer on the first electrode, a hole transport layer on the hole injection layer, and an electron blocking layer on the hole transport layer, and
at least one of the hole injection layer, the hole transport layer, or the electron blocking layer comprises the heterocyclic compound.

11. The light emitting element of claim 1, wherein the heterocyclic compound is represented by any one selected from among compounds of Compound Group 1: 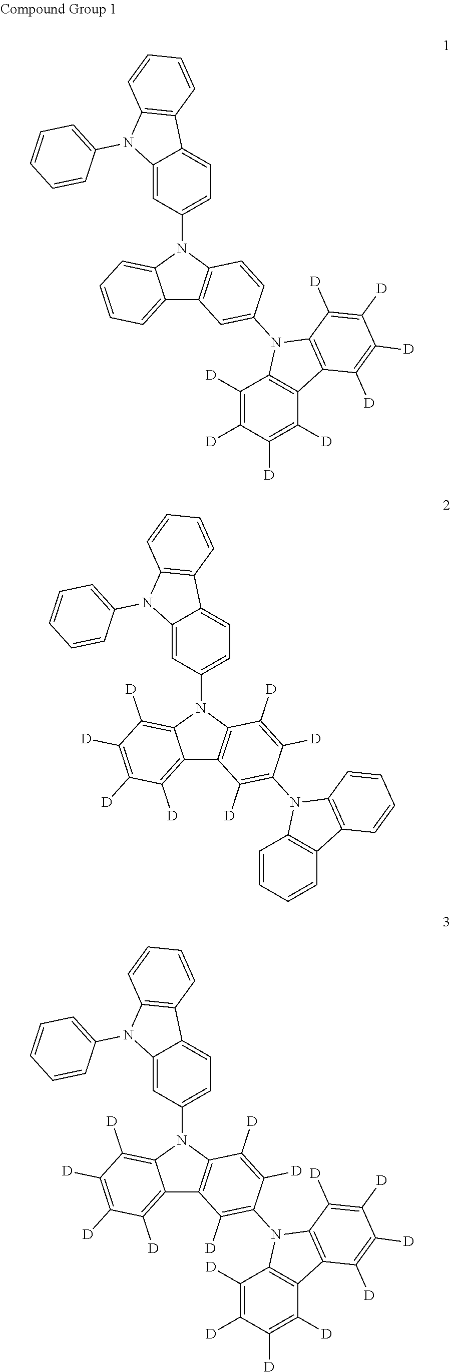 [SMILES: [2H]c1c([2H])c([2H])c2c(c1[2H])c1c([2H])c([2H])c([2H])c([2H])c1n2-c1ccc2c(c1)c1ccccc1n2-c1ccc2c3ccccc3n(-c3ccccc3)c2c1 ; [2H]c1c([2H])c([2H])c2c(c1[2H])c1c([2H])c(-n3c4ccccc4c4ccccc43)c([2H])c([2H])c1n2-c1ccc2c3ccccc3n(-c3ccccc3)c2c1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc2c3ccccc3n(-c3ccccc3)c2c1] 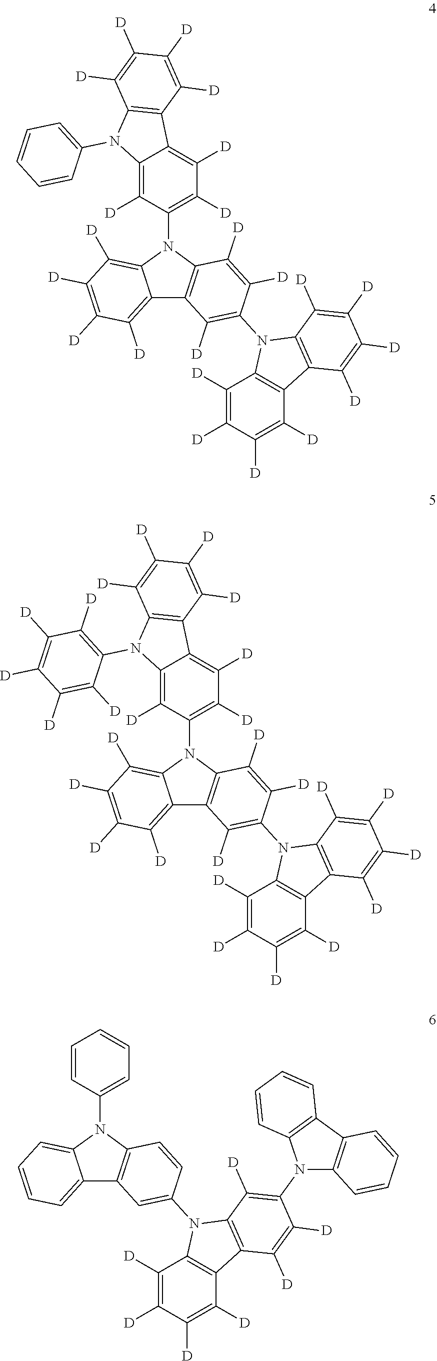 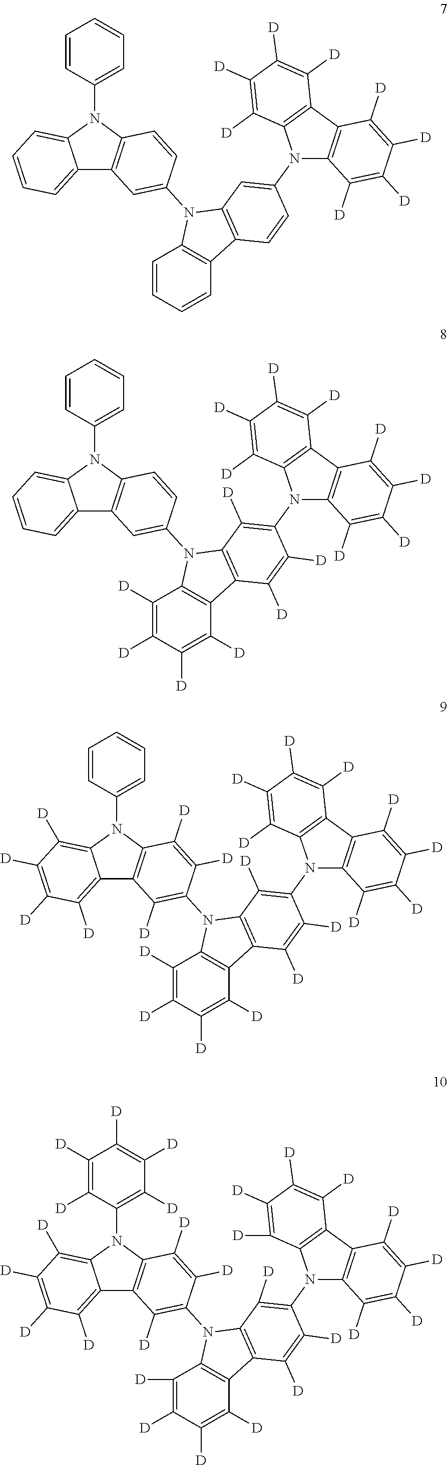 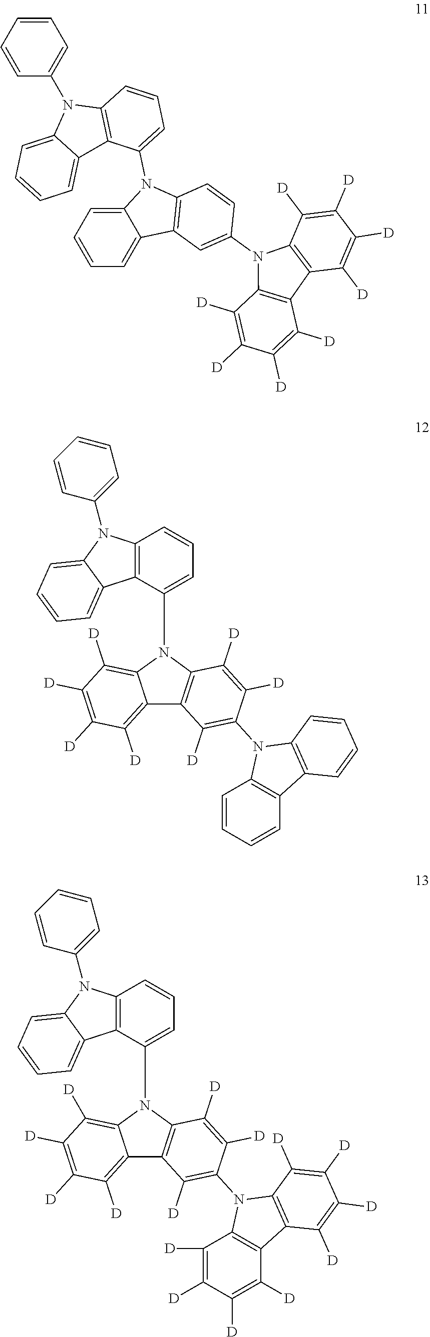 [SMILES: [2H]c1c([2H])c([2H])c2c(c1[2H])c1c([2H])c([2H])c([2H])c([2H])c1n2-c1ccc2c(c1)c1ccccc1n2-c1cccc2c1c1ccccc1n2-c1ccccc1 ; [2H]c1c([2H])c([2H])c2c(c1[2H])c1c([2H])c(-n3c4ccccc4c4ccccc43)c([2H])c([2H])c1n2-c1cccc2c1c1ccccc1n2-c1ccccc1 ; [2H]c1c([2H])c([2H])c2c(c1[2H])c1c([2H])c([2H])c([2H])c([2H])c1n2-c1c([2H])c([2H])c2c(c1[2H])c1c([2H])c([2H])c([2H])c([2H])c1n2-c1cccc2c1c1ccccc1n2-c1ccccc1] 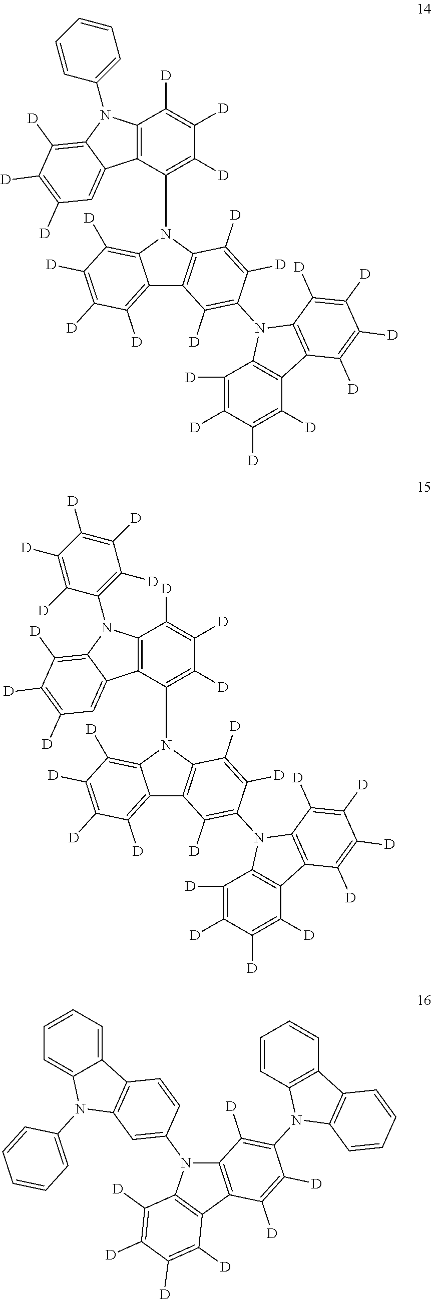 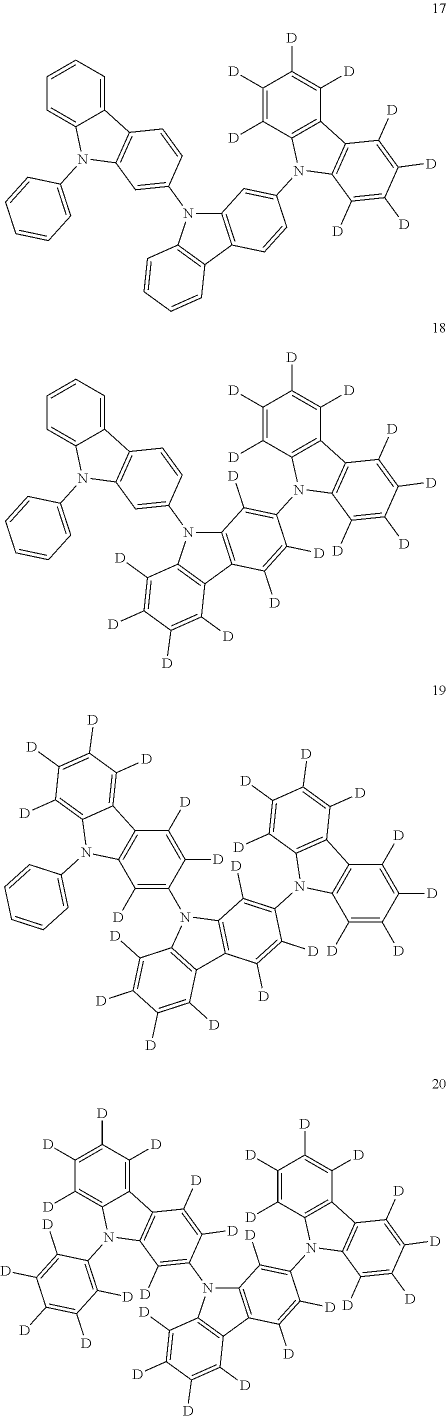 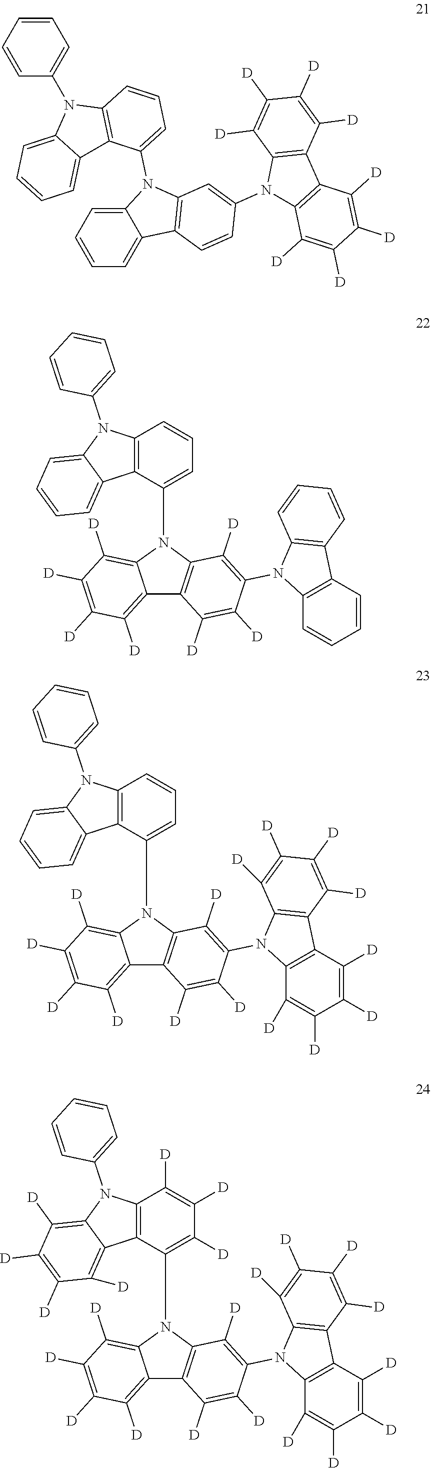 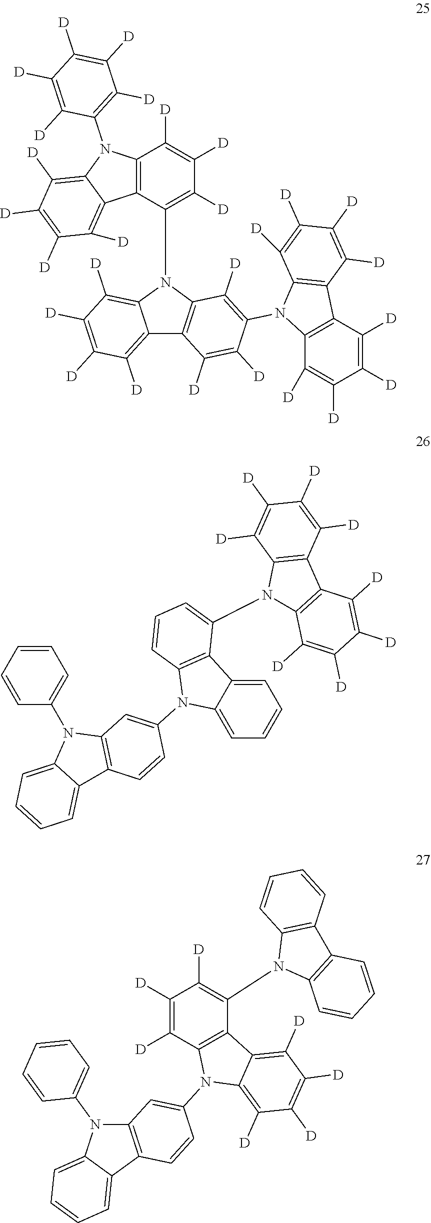 [SMILES: [2H]c1c([2H])c([2H])c(-n2c3c([2H])c([2H])c([2H])c([2H])c3c3c(-n4c5c([2H])c([2H])c([2H])c([2H])c5c5c([2H])c([2H])c(-n6c7c([2H])c([2H])c([2H])c([2H])c7c7c([2H])c([2H])c([2H])c([2H])c76)c([2H])c54)c([2H])c([2H])c([2H])c32)c([2H])c1[2H] ; [2H]c1c([2H])c([2H])c2c(c1[2H])c1c([2H])c([2H])c([2H])c([2H])c1n2-c1cccc2c1c1ccccc1n2-c1ccc2c3ccccc3n(-c3ccccc3)c2c1 ; [2H]c1c([2H])c([2H])c2c(c1[2H])c1c(-n3c4ccccc4c4ccccc43)c([2H])c([2H])c([2H])c1n2-c1ccc2c3ccccc3n(-c3ccccc3)c2c1] 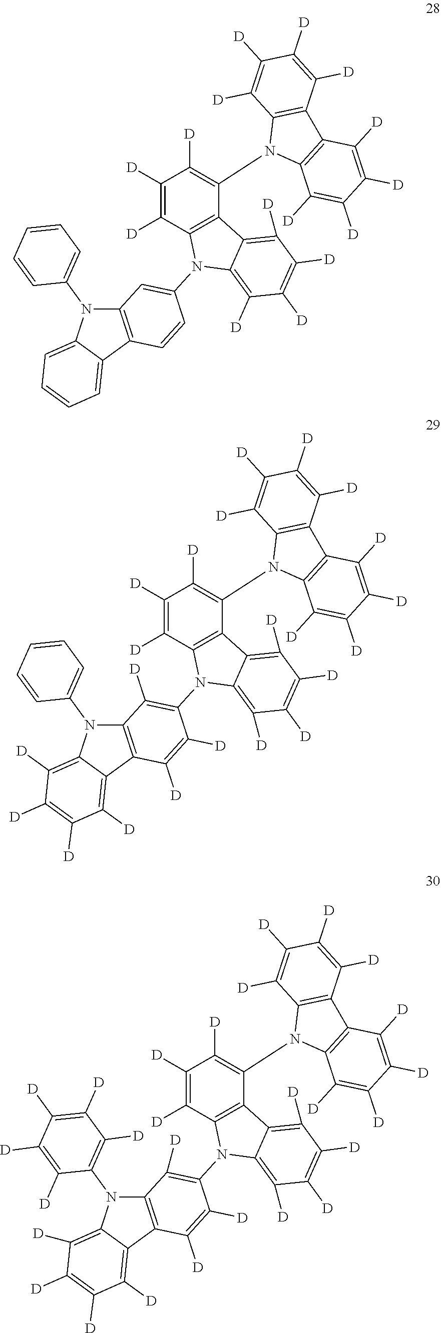 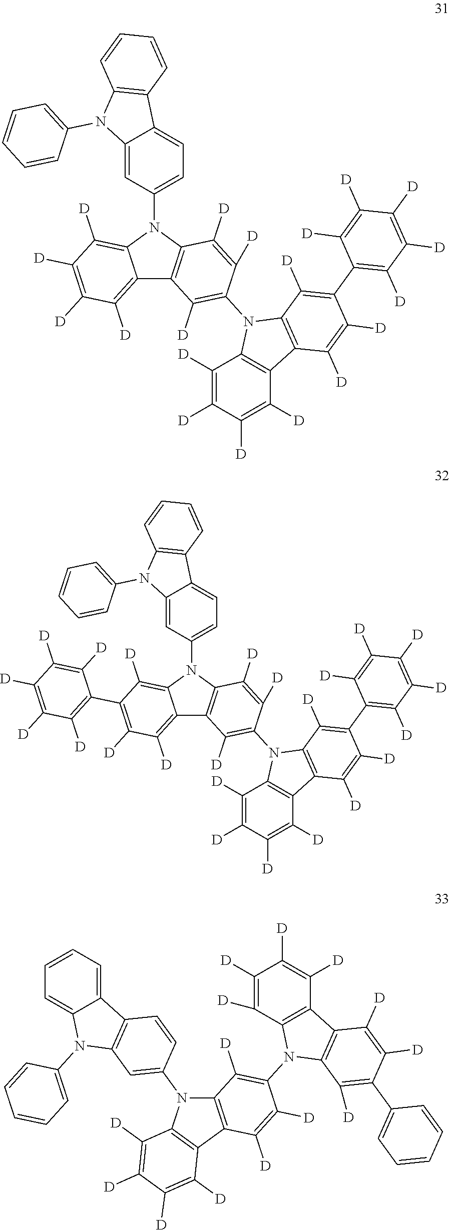 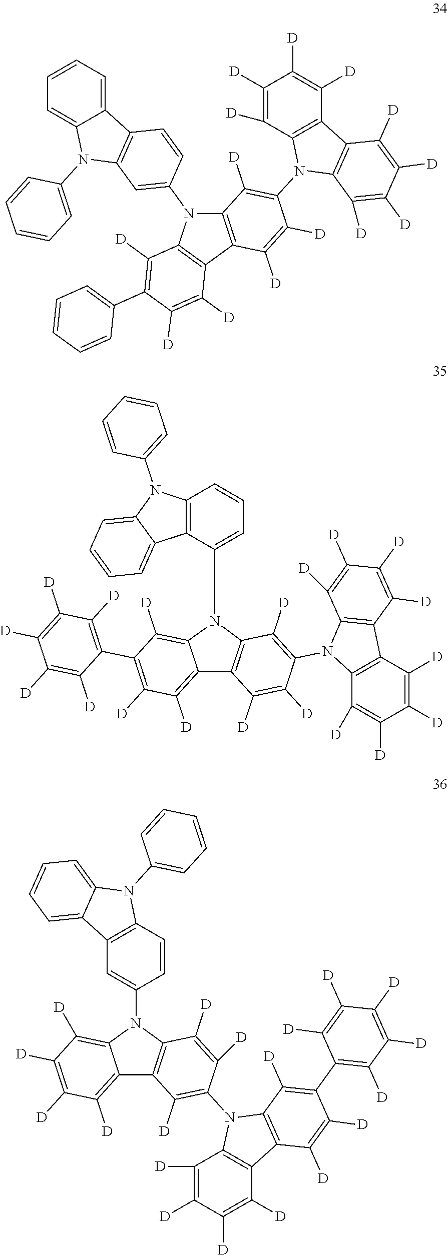 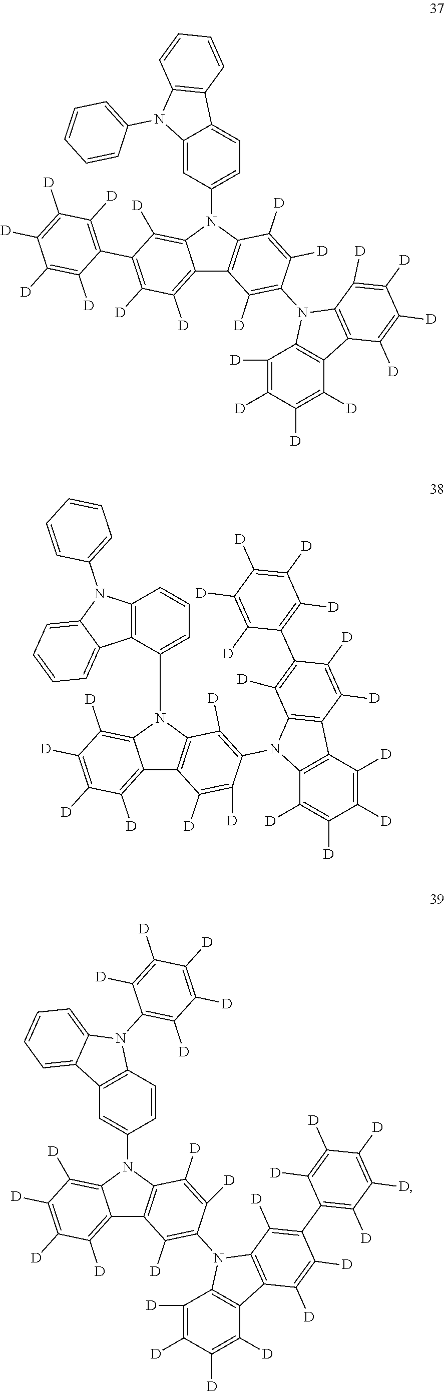
wherein in Compound Group 1, D is a deuterium atom.

12. A heterocyclic compound represented by Formula 1: 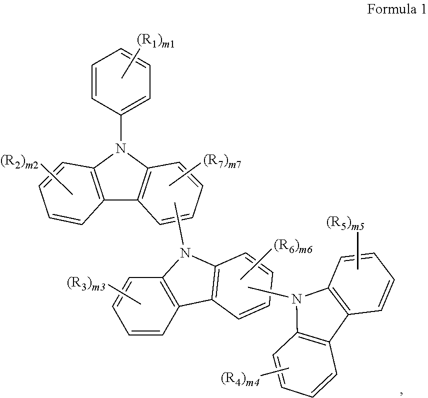
wherein in Formula 1,
m1 is an integer of 1 to 5,
m2 to m5 are each independently an integer of 1 to 4,
m6 and m7 are each independently an integer of 1 to 3,
at least one selected from among R₁ to R₇ is a deuterium atom, and
remaining ones of R₁ to R₇ are each independently a hydrogen atom or a substituted or unsubstituted aryl group having 6 to 30 ring-forming carbon atoms.

13. The heterocyclic compound of claim 12, wherein Formula 1 is represented by any one selected from among Formulae 1-1 to 1-3: 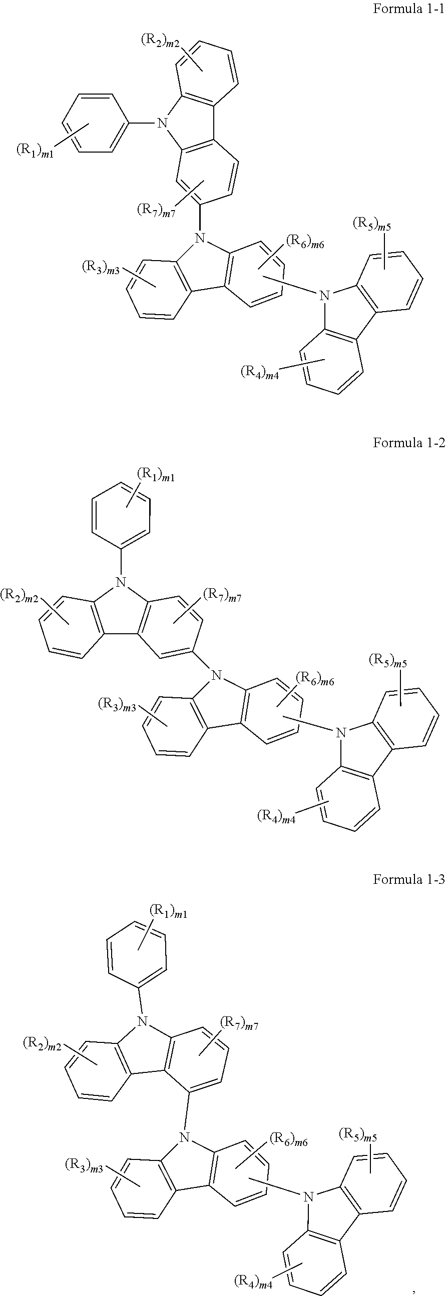
wherein in Formulae 1-1 to 1-3,
m1 to m7 and R₁ to R₇ are the same as defined in Formula 1.

14. The heterocyclic compound of claim 13, wherein Formula 1-1 is represented by any one selected from among Formulae 1-1A to 1-1C: 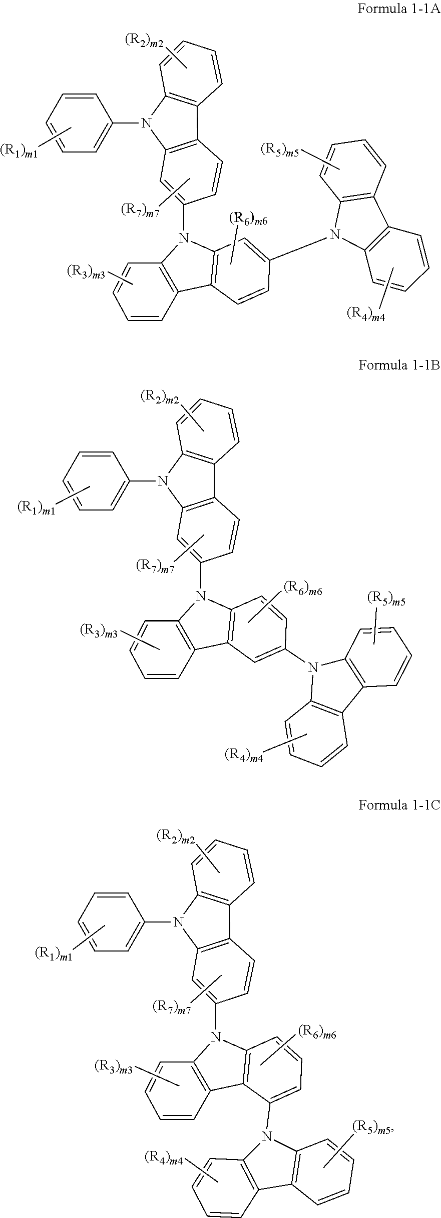
wherein in Formulae 1-1 Å to 1-1C,
m1 to m7 and R₁ to R₇ are the same as defined in Formula 1-1.

15. The heterocyclic compound of claim 13, wherein Formula 1-2 is represented by Formula 1-2A or Formula 1-2B: 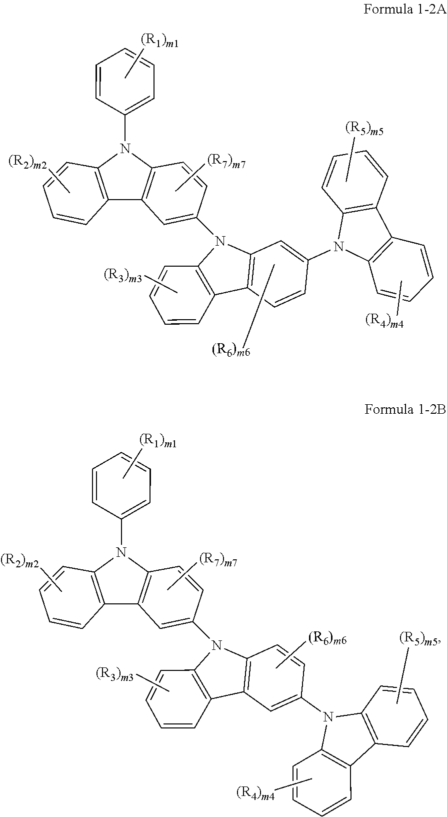
wherein in Formulae 1-2A and 1-2B,
m1 to m7 and R₁ to R₇ are the same as defined in Formula 1-2.

16. The heterocyclic compound of claim 13, wherein Formula 1-3 is represented by Formula 1-3A or Formula 1-3B: 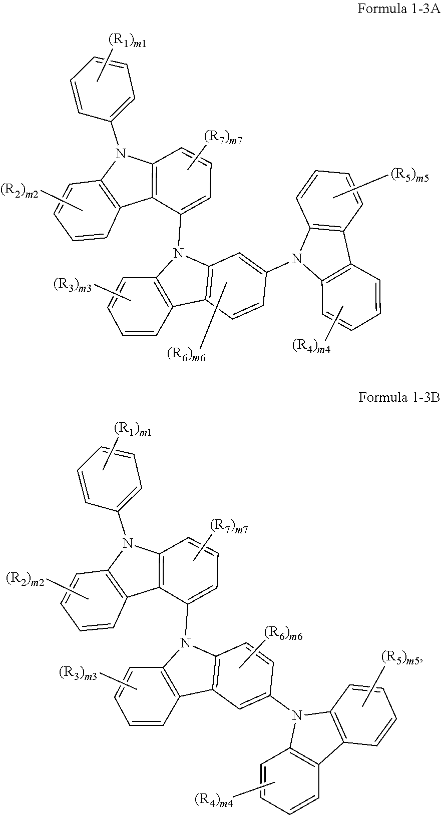
wherein in Formulae 1-3A and 1-3B,
m1 to m7 and R₁ to R₇ are the same as defined in Formula 1-3.

17. The heterocyclic compound of claim 12, wherein Formula 1 is represented by Formula 1-X1 or Formula 1-X2: 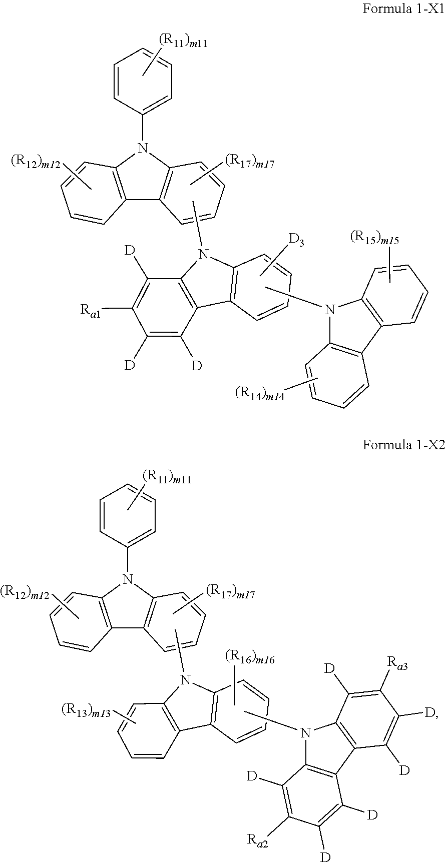
wherein in Formula 1-X1,
Rₐ₁ is a deuterium atom or a substituted or unsubstituted phenyl group,
m14 and m15 are each independently an integer of 1 to 4, and
R₁₄ and R₁₅ are each independently a hydrogen atom, a deuterium atom, or a substituted or unsubstituted phenyl group,
wherein in Formula 1-X2,
Rₐ₂ and Rₐ₃ are each independently a deuterium atom or a substituted or unsubstituted phenyl group,
m13 is an integer of 1 to 4,
m16 is an integer of 1 to 3, and
R₁₃ and R₁₆ are each independently a hydrogen atom, a deuterium atom, or a substituted or unsubstituted phenyl group, and
wherein in Formulae 1-X1 and 1-X2,
D is a deuterium atom,
m11 is an integer of 1 to 5,
m12 is an integer of 1 to 4,
m17 is an integer of 1 to 3, and
R₁₁, R₁₂, and R₁₇ are each independently a hydrogen atom, a deuterium atom, or a substituted or unsubstituted phenyl group.

18. The heterocyclic compound of claim 12, wherein Formula 1 is represented by Formula 1-X3: 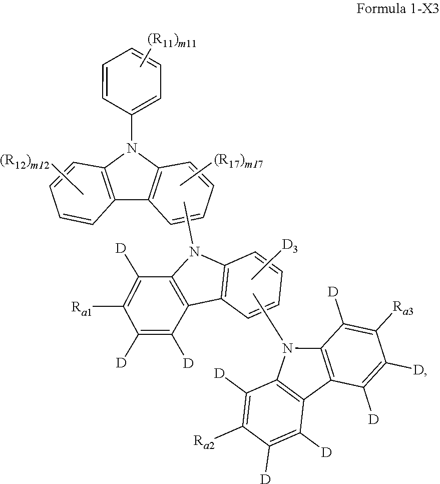
wherein in Formula 1-X3,
D is a deuterium atom,
m11 is an integer of 1 to 5,
m12 is an integer of 1 to 4,
m17 is an integer of 1 to 3,
R₁₁, R₁₂, and R₁₇ are each independently a hydrogen atom, a deuterium atom, or a substituted or unsubstituted phenyl group, and
Rₐ₁ to Rₐ₃ are each independently a deuterium atom or a substituted or unsubstituted phenyl group.

19. The heterocyclic compound of claim 18, wherein in Formula 1-X3, at least one selected from among Rₐ₁ to Rₐ₃ is an unsubstituted phenyl group or a phenyl group substituted with a deuterium atom.

20. The heterocyclic compound of claim 12, wherein Formula 1 is represented by any one selected from among compounds of Compound Group 1: 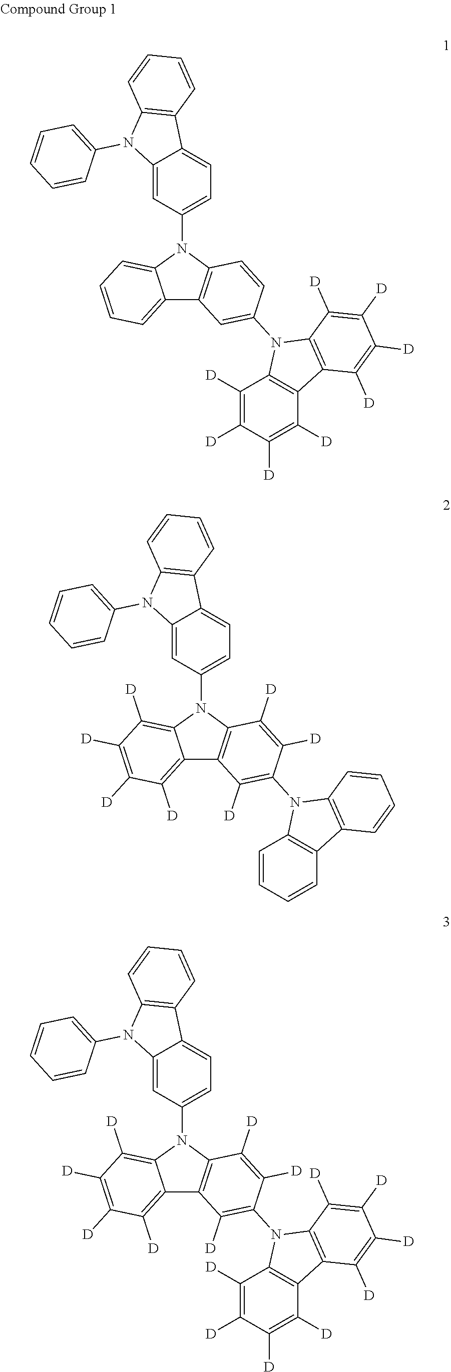 [SMILES: [2H]c1c([2H])c([2H])c2c(c1[2H])c1c([2H])c([2H])c([2H])c([2H])c1n2-c1ccc2c(c1)c1ccccc1n2-c1ccc2c3ccccc3n(-c3ccccc3)c2c1 ; [2H]c1c([2H])c([2H])c2c(c1[2H])c1c([2H])c(-n3c4ccccc4c4ccccc43)c([2H])c([2H])c1n2-c1ccc2c3ccccc3n(-c3ccccc3)c2c1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc2c3ccccc3n(-c3ccccc3)c2c1] 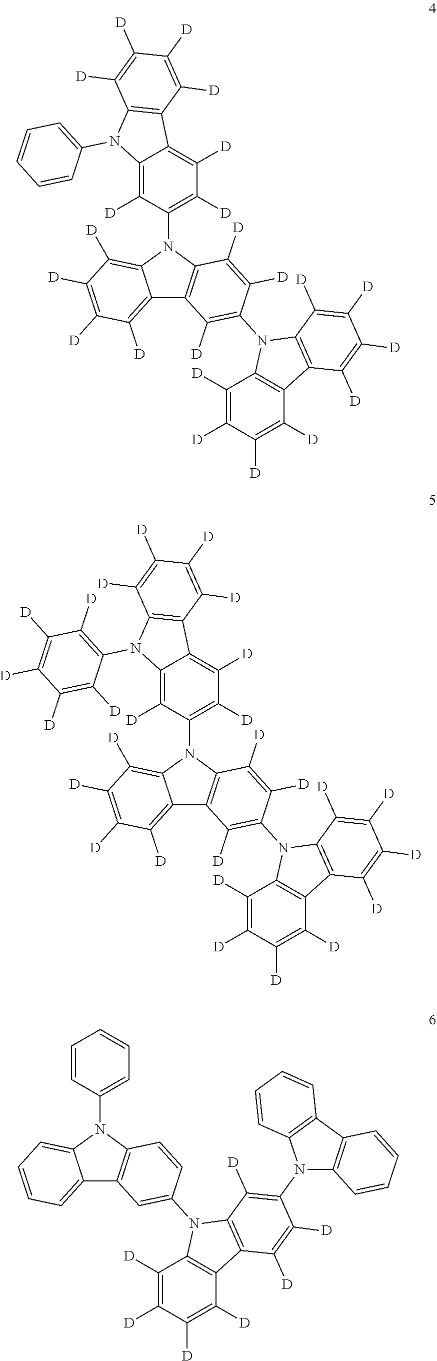 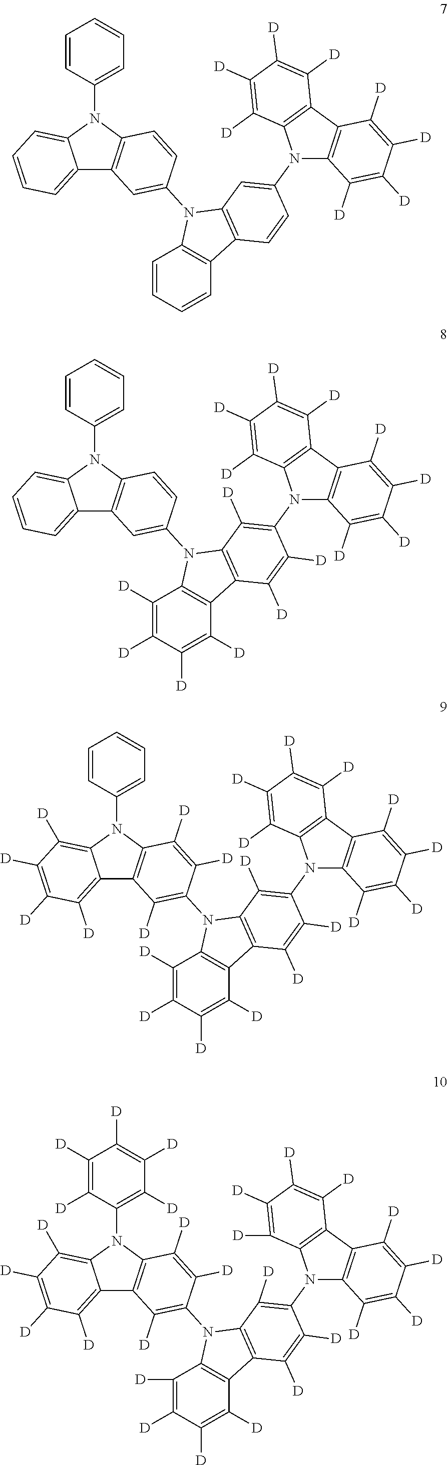 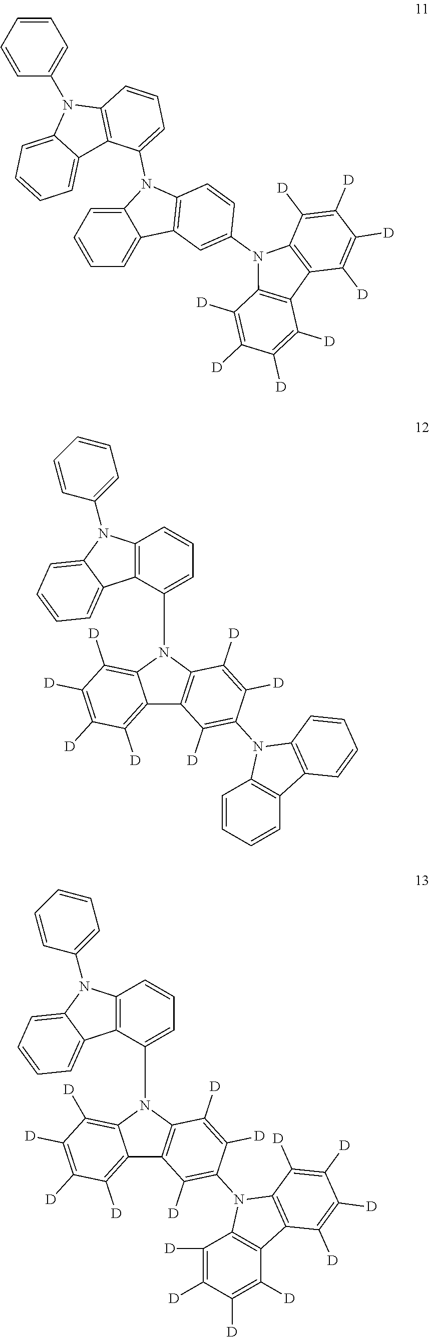 [SMILES: [2H]c1c([2H])c([2H])c2c(c1[2H])c1c([2H])c([2H])c([2H])c([2H])c1n2-c1ccc2c(c1)c1ccccc1n2-c1cccc2c1c1ccccc1n2-c1ccccc1 ; [2H]c1c([2H])c([2H])c2c(c1[2H])c1c([2H])c(-n3c4ccccc4c4ccccc43)c([2H])c([2H])c1n2-c1cccc2c1c1ccccc1n2-c1ccccc1 ; [2H]c1c([2H])c([2H])c2c(c1[2H])c1c([2H])c([2H])c([2H])c([2H])c1n2-c1c([2H])c([2H])c2c(c1[2H])c1c([2H])c([2H])c([2H])c([2H])c1n2-c1cccc2c1c1ccccc1n2-c1ccccc1] 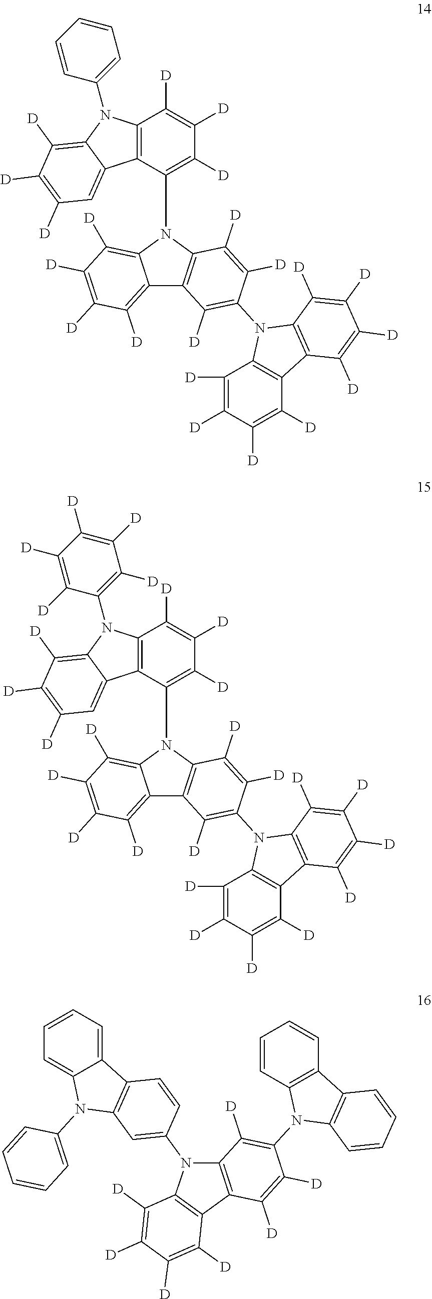 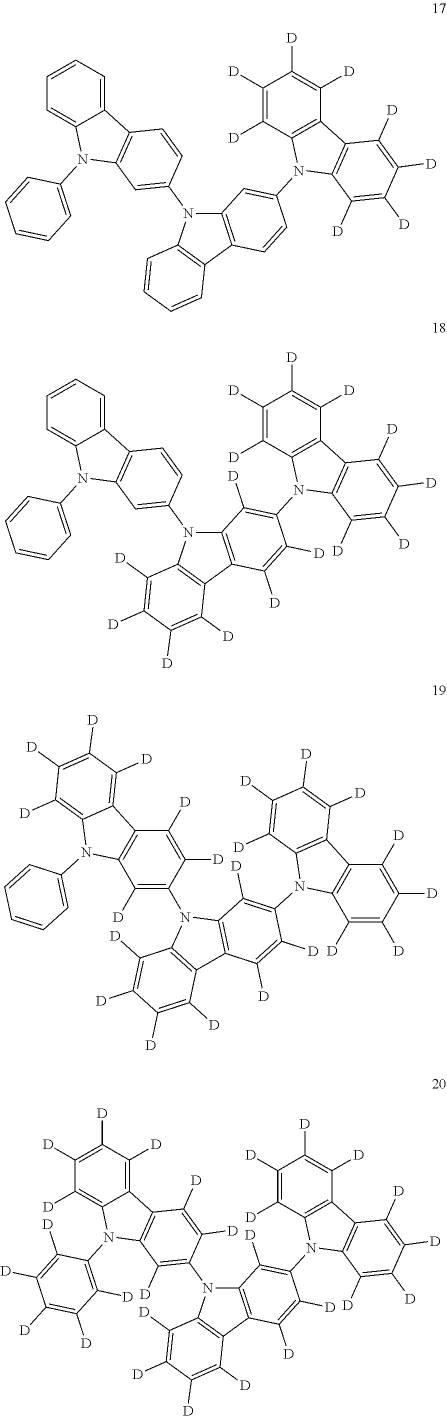 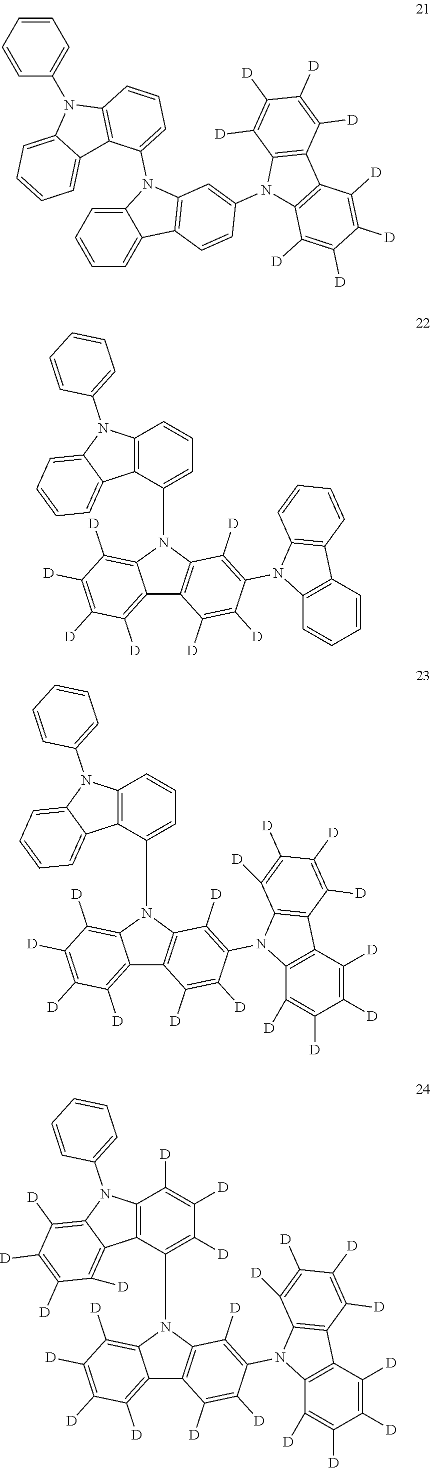 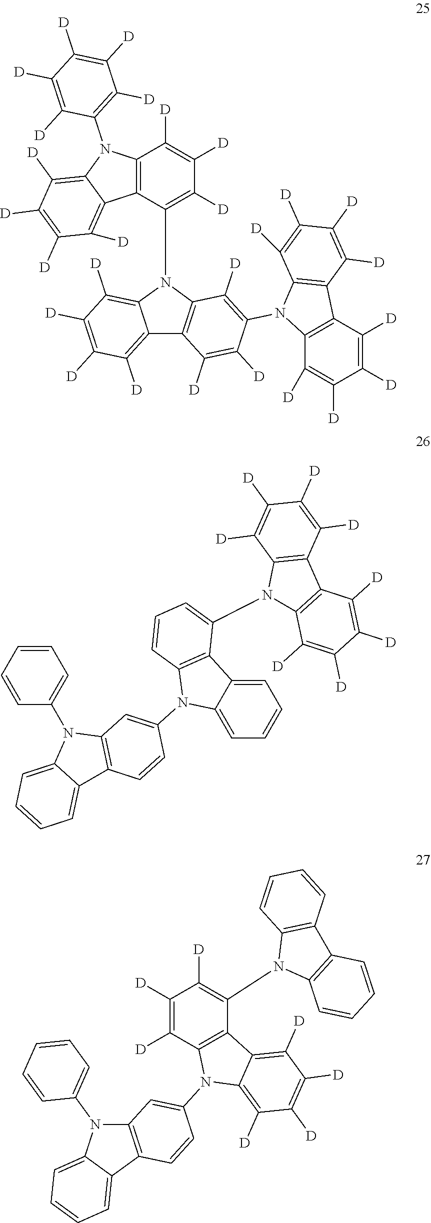 [SMILES: [2H]c1c([2H])c([2H])c(-n2c3c([2H])c([2H])c([2H])c([2H])c3c3c(-n4c5c([2H])c([2H])c([2H])c([2H])c5c5c([2H])c([2H])c(-n6c7c([2H])c([2H])c([2H])c([2H])c7c7c([2H])c([2H])c([2H])c([2H])c76)c([2H])c54)c([2H])c([2H])c([2H])c32)c([2H])c1[2H] ; [2H]c1c([2H])c([2H])c2c(c1[2H])c1c([2H])c([2H])c([2H])c([2H])c1n2-c1cccc2c1c1ccccc1n2-c1ccc2c3ccccc3n(-c3ccccc3)c2c1 ; [2H]c1c([2H])c([2H])c2c(c1[2H])c1c(-n3c4ccccc4c4ccccc43)c([2H])c([2H])c([2H])c1n2-c1ccc2c3ccccc3n(-c3ccccc3)c2c1] 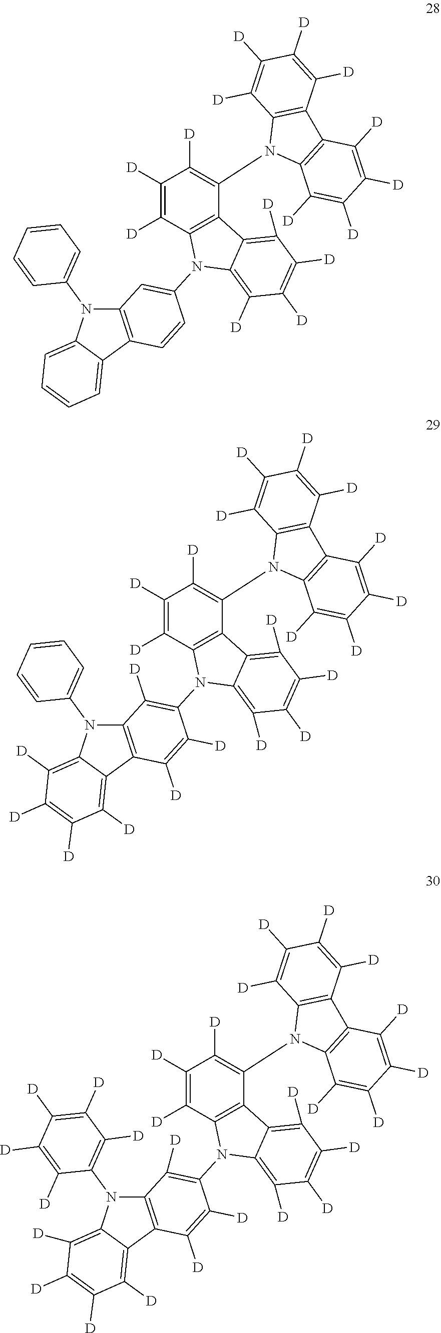 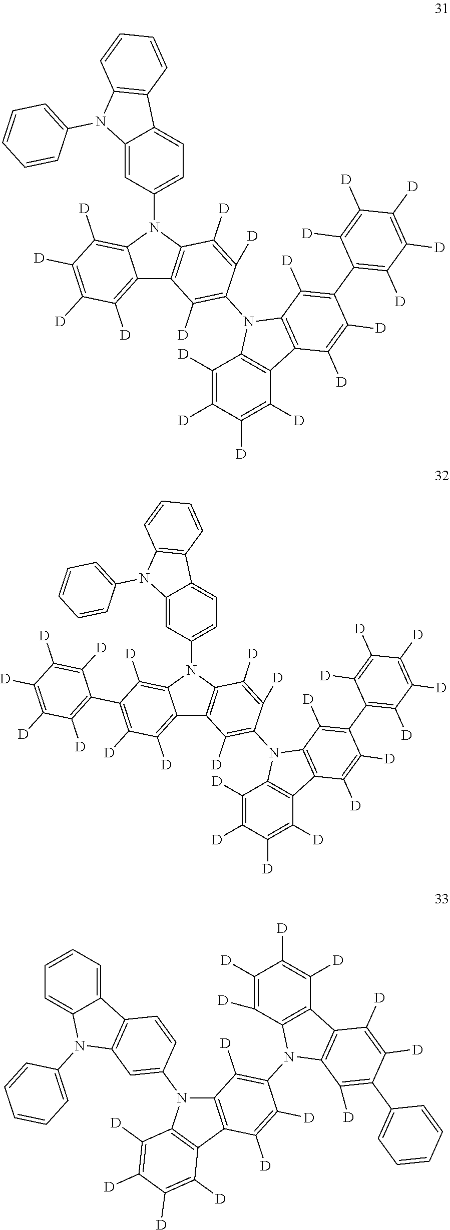 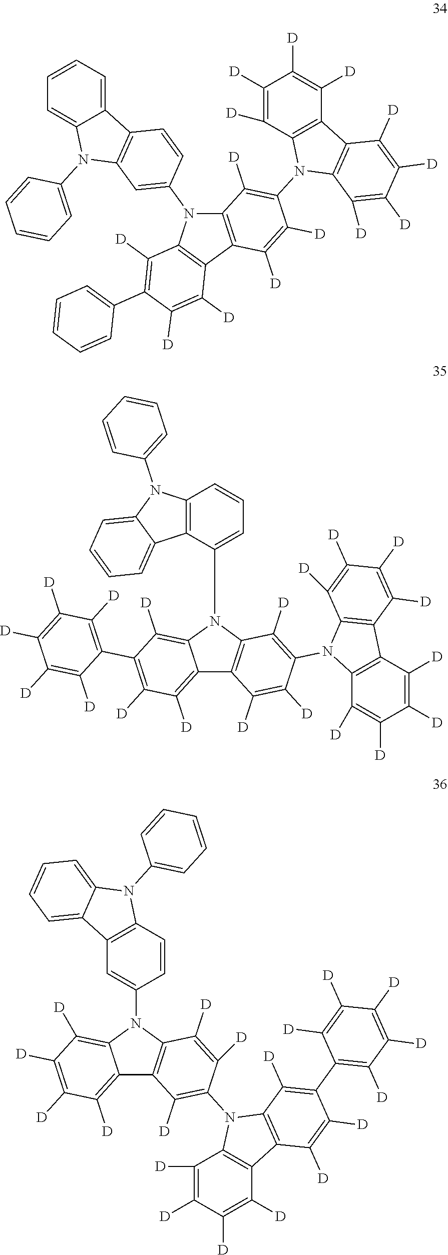 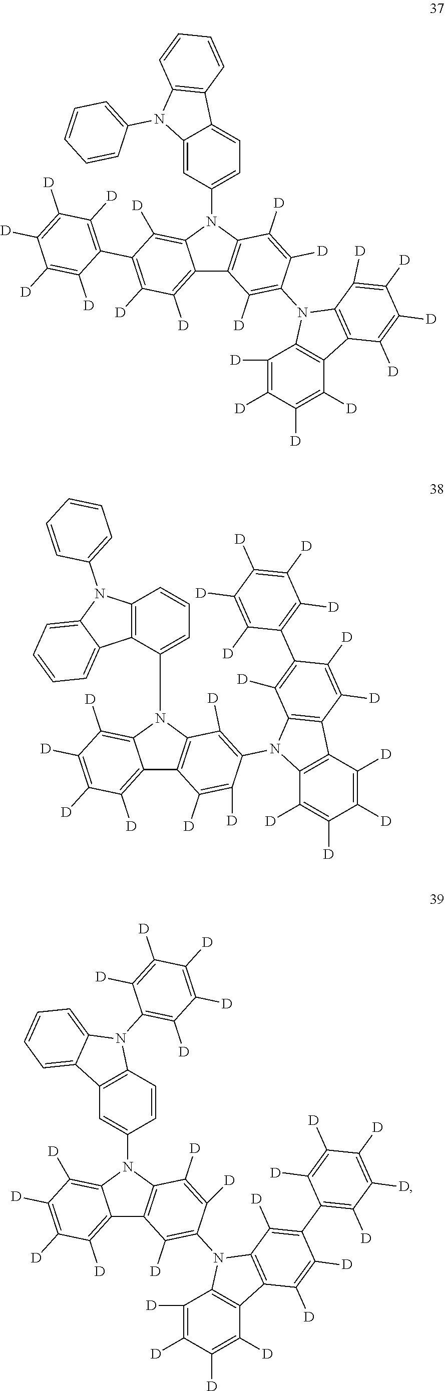
and
wherein in Compound Group 1, D is a deuterium atom.
